(19) United States
(12) Patent Application Publication
(10) Pub. No.: US 2026/0098258 A1
(43) Pub. Date: Apr. 9, 2026

(54) USE OF DNA ORIGAMI NANOSTRUCTURES FOR MOLECULAR INFORMATION BASED DATA STORAGE SYSTEMS

(71) Applicant: BATTELLE MEMORIAL INSTITUTE, Columbus, OH (US)

(72) Inventors: Cherry Gupta, Columbus, OH (US); Craig M. Bartling, Powell, OH (US); Rachel R. Spurbeck, Columbus, OH (US); Anthony D. Duong, Columbus, OH (US); James Ha, Columbus, OH (US); Miguel D. Pedrozo, Columbus, OH (US); Nickolas R. Andrioff, Columbus, OH (US)

(21) Appl. No.: 19/319,178

(22) Filed: Sep. 4, 2025

Related U.S. Application Data
(63) Continuation of application No. 18/972,564, filed on Dec. 6, 2024, now Pat. No. 12,441,996.
(60) Provisional application No. 63/607,741, filed on Dec. 8, 2023.

(51) Int. Cl. C12N 15/10; C12Q 1/6869

(52) U.S. Cl. CPC C12N 15/1093; C12Q 1/6869

(57) ABSTRACT

The present disclosure is directed to compositions and methods that use the principles of DNA origami to package and archive data stored in multiple indexed DNA oligonucleotides. These structures allow for selective physical data access and retrieval from a molecular pool of DNA origami (DNAO) nanostructures comprising the data bearing oligonucleotides.

### CROSS REFERENCE TO RELATED APPLICATIONS

This application is a continuation of U.S. patent application Ser. No. 18/972,564, filed Dec. 6, 2024 which claims the benefit and priority to U.S. Provisional Application Ser. No. 63/607,741 filed on Dec. 8, 2023, the entire disclosure of which is incorporated herein by reference.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as an 865 kilobytes 865,000 byte xml file named “416751.xml,” created on Dec. 2, 2024.

### BACKGROUND

The shift to digital systems for the creation, transmission and storage of information has led to increasing complexity in archiving data, requiring active, ongoing maintenance of the digital media. DNA is an attractive medium for information storage because of its capacity for high density information encoding, longevity under easily-achieved conditions and proven track record as an information bearer. Thus, relative to the solid state storage media, DNA provides superior data density and durability. For example, data stored in the DNA sequence is significantly more dense than the most compact solid-state hard drive and significantly more durable than the most stable magnetic tapes. In addition, DNA's four-letter nucleotide code offers a suitable coding environment that can be leveraged like the binary digital code used by computers and other electronic devices to represent any letter, digit, or other character. Furthermore, studies show that DNA properly encapsulated with a salt remains stable for decades at room temperature and should last much longer in the controlled environs of a data center. In addition, DNA doesn't require maintenance, and files stored in DNA are easily copied for negligible cost.

Current molecular data archival systems suffer from one or more deficiencies including the failure to efficiently allow selective access to specific data sets (random access), and/or the failure to allow repeated information access without loss in information fidelity. More particularly, current approaches for achieving random access, which avoid sequencing of the entire pool include:
(1) polymerase chain reaction (PCR) based amplification to selectively enrich a sub-pool over the background by added address-specific primers; and
(2) physical separation of the desired sub-pool through the use of magnetic beads or fluorescent based sorting (FACS). While the PCR method of random access scales well to a pool capacity of 17 exabytes/gram, it necessitates a rigorous design of the primers or the use of a hierarchical addressing system to achieve the specificity at scale. Moreover, these primer-based addressing systems irreversibly remove oligonucleotides from the pool and are incompatible with common storage approaches, necessitating the removal and re-embedding of the encoding DNA into the storage pool for each random-access operation.

In accordance with one embodiment of the present invention a storage system is provided that solves these challenges by using DNA Origami (DNAO) techniques to package the data encoded DNA strands. This approach will act both as a filing and addressing system for storing DNA molecules and will allow for a straightforward single-step method for random access without the need for removing the data containing oligonucleotides from the storage pool.

### SUMMARY

In accordance with one embodiment, the present disclosure is directed to compositions and methods that allow for selective physical data access and retrieval from a molecular pool. Current data molecular data archival data systems do not allow for selective access to specific data sets (random access), high storage density and/or repeated information access without loss in information fidelity. One aspect of the present disclosure is directed to a method for DNA data archiving that uses the principles of DNA origami to package and archive data stored in multiple indexed DNA oligonucleotides into individual DNA origami (DNAO) nanostructures (named “DNAFiles” herein) for precise organization, greater stability, and ease of data retrieval.

Current strategies for data retrieval that employ polymerase chain reaction (PCR) based random access, rely on additional separation steps which introduces complexity and an irreversible loss of the retrieved data. The presently disclosed methods use a DNAFile system, wherein a single-step retrieval is used to address the gap in traditional molecular information archival systems, and thus accelerates the potential access time and increases the stability of DNA data storage.

In accordance with one embodiment of the present disclosure, a library of DNAFiles is provided wherein the library comprises a plurality of origami folded DNAFiles, where each of the DNAFiles comprises a single stranded DNA scaffold and a plurality of single stranded DNA staple oligonucleotides that bind through complementary base pairing with two non-contiguous segments of the DNA scaffold, wherein said staple oligonucleotides cause the DNA scaffold to reversibly fold into a two or three dimensional shape. The DNAFiles further comprised a plurality of data oligonucleotides that comprise a nucleic acid sequence complementary to the DNA scaffold and a nucleic acid sequence that is non-complementary to the DNA scaffold wherein the non-complementary nucleic acid sequence encodes digital information. In one embodiment the nucleic acid sequences that encodes digital information further comprise a first and second primer binding sequence located at the respective 5′ and 3′ ends of each nucleic acid sequence encoding digital information to allow PCR amplification of the nucleic acid sequence encoding digital information. In one embodiment the first and second primer binding sequences located at the respective 5′ and 3′ ends of each data oligonucleotide, wherein both the 5′ end of the data oligonucleotide and the 3′ end of the data oligonucleotide are non-complementary to the DNA scaffold. In one embodiment each of the individual DNAFiles differ from one another based on the nucleic acid sequence of the staple oligonucleotides and/or the data oligonucleotides bound to the DNA scaffold of each DNAFile.

In a further embodiment Applicant has discovered that libraries of DNAFiles comprising data oligonucleotides projecting away from the scaffold strand induces a degree of aggregation correlated to the % occupancy (see FIG. 2). However, one-sided occupancy substantially reduced muti-order structures. Accordingly, in one embodiment DNAFiles are prepared comprising a plurality of data oligonucleotides bound to the DNA scaffold and projecting away from only one side of the DNA scaffold, and generally in only one direction, optionally all overhang regions projecting away from only one side of the DNA scaffold within an angle about 80 to 90 degrees relative to the DNA scaffold surface.

Use of the first and second primer binding sequences located at the respective 5′ and 3′ ends of each data oligonucleotide allows for amplification of the entire data oligonucleotide and reconstitution of the original DNAFile and sequence analysis of the generated amplicons to retrieve the data encoded by the data oligonucleotide. This process provides a check for encoding fidelity/corruption data based on the 2D/3D structure of the origami DNA (if there is an error in the base sequence, the structure will not fold properly); provides an option of labelling individual DNAFiles with unique DNA barcodes for identifying single DNAFiles and separate them from other DNAFiles of the library for accessing the data of specific portions of a library of stored data; and allows for rapid recovery of the original DNA File after accessing the data, through reannealing the nucleic acid sequences to reconstitute the DNAFile and isolating the DNAFile by size separation (i.e. gel electrophoresis, or size exclusion chromatography).

Advantages of the present system of using DNAFiles include:
a) Data can be stored at multiple levels: in the multiple smaller oligonucleotide staple strands, in the data oligonucleotides, in the longer scaffold strand or in the 3D folded structure itself allowing for greater storage flexibility and hierarchical organization.
b) Physical encryption keys will lock or unlock targeted DNAFiles for storage, readout, or tamper-prevention.
c) Data exists in a closed-packed configuration that has higher stability than regular duplex DNA.
d) Data are easily addressable by inclusion of staple overhangs/bar codes that can be base-paired to externally added functionalized oligonucleotides for physical separation if needed.

In accordance with one embodiment a library comprising a plurality of origami folded DNA files (DNAFile) is provided, wherein each DNAFile comprises a single stranded scaffold DNA, a plurality of staple oligonucleotides, and a plurality of data oligonucleotides, wherein a unique set of data is stored within the sequences of the scaffold DNA, the data oligonucleotides and/or staple oligonucleotides of the DNAFiles. In one embodiment the data is stored solely within the sequence of the data oligonucleotides. In one embodiment the individual DNAFiles differ from one another based on the nucleic acid sequence of the data oligonucleotides bound to the DNA scaffold of each DNAFile, and optionally also differ from one another based on the nucleotide sequence of the respective DNA scaffold and staple oligonucleotides of each DNAFile. Each DNAFile comprises a single stranded DNA scaffold; and a plurality of single stranded DNA staple oligonucleotides, wherein the staple oligonucleotides have a length less than 10%, 5% or 1% of the DNA scaffold and bind through complementary base pairing with non-contiguous nucleic acid sequences of the DNA scaffold, further wherein said staple oligonucleotides cause the DNA scaffold to reversibly fold into a two or three dimensional shape. In one embodiment the nucleic acids of the DNA scaffold, the data oligonucleotides and/or one or more of said staple oligonucleotides comprise nucleic acid sequences that encode digital information, optionally wherein only the data oligonucleotides comprise nucleic acid sequences that encode digital information. In one embodiment the data oligonucleotides comprise: 1) a nucleic acid sequence complementary to a nucleic acid sequence of the single stranded DNA scaffold, 2) a nucleic acid sequence that encodes digital information, and 3) a first and second primer binding sequence, wherein the first primer binding sequence is 5′ to the digital information encoding nucleic acid sequence, and the second primer binding sequence is 3′ to the digital information encoding nucleic acid sequence.

In one embodiment the staple oligonucleotides and the data oligonucleotides of the individual DNAFiles have a length of about 30 to about 200 or about 50 to about 150 nucleotides or about 30 to about 100 nucleotides or about 80 or 100 nucleotides in length. The staple oligonucleotides comprise a first and second sequence that are complementary to non-contiguous sequences present on the scaffold, such that upon binding of the staple oligonucleotide to the DNA scaffold, the DNA scaffold is folded. The data oligonucleotides comprise a sequence that is complementary to the DNA scaffold DNA and a sequence that is non-complementary to said DNA scaffold (i.e., an “overhang”), wherein the non-complementary region comprises nucleic acid sequences that encode digital information. In one embodiment the overhang region of the data oligonucleotide is at least 50 nucleotides in length and up to 180 nucleotides in length. In one embodiment the data oligonucleotides further comprise primer binding sequences and optionally barcoding sequences. In accordance with one embodiment each data oligonucleotide is provided with a primer binding sequence at the 5′ and the 3′ end of the data oligonucleotide to allow for PCR amplification of the entire data oligonucleotides upon release from the DNA scaffold of the DNAFile. In one embodiment the two primer binding sequence flank the non-complementary nucleic acid sequences that encode digital information, wherein a first primer binding sequence is located at the 5′ terminus of the non-complementary nucleic acid sequence and a second primer binding sequence is located at the 3′ terminus of the non-complementary nucleic acid sequence, and said sequence complementary to the DNA scaffold is located 5′ to the first primer binding sequence or 3′ to the second primer binding sequence. In one embodiment each data oligonucleotide of a DNAFile is provided with the same pair of primer binding sequence located at the respective 3′ and 5′ ends of 1) each data oligonucleotide or 2) the non-complementary nucleic acid sequence of each data oligonucleotide. In one embodiment the primer binding sequences are 10 to 20 nucleotides in length and the non-complementary nucleic acid sequence is about 10 to 60 nucleotides in length. In one embodiment the primer binding sequences are 10 to 20 nucleotides in length and the non-complementary nucleic acid sequence is about 40 to 160 nucleotides in length. In one embodiment the primer binder sequences differ between the data oligonucleotides of one DNAFile relative to the primer binding sequence of the data oligonucleotides of other DNAFiles of the library of DNAFiles. In one embodiment a subset of the data oligonucleotides of an individual DNAFile can comprises different primer binding sequence relative to one another. In one embodiment the 3′ end of the non-complementary region/overhang of said data oligonucleotide comprises a poly A or poly T extension. In one embodiment, at least a portion of the non-complementary region of said data oligonucleotides is designed to form a hairpin structure.

In one embodiment the DNAFiles of the present invention are folded by the staple oligonucleotides into a predetermined two dimensional or three dimensional shape having a plurality of exterior surfaces. In one embodiment the data oligonucleotides are bound to only one exterior surface of the two dimensional or three dimensional shaped DNA scaffold, wherein the non-complementary sequences of the data oligonucleotides (overhang) project away from the DNA scaffold in approximately the same direction. In one embodiment the staple oligonucleotides fold the DNA scaffold into the shape of a multi-layered sheet. In one embodiment the multi-layered sheet comprises two sheets of origami folded DNA layered on top of each other in either a parallel or ani-parallel orientation, wherein the multilayered sheet has a top surface and a bottom surface. In one embodiment the data oligonucleotides are bound only to the top surface, wherein the non-complementary sequences of the data oligonucleotides (overhang) project away from the DNA scaffold in approximately the same direction (each projecting away at an angle within 70 to 90 degrees or within 80 to 90 degrees). In one embodiment the density of the data oligonucleotides can be varied from a low density (approximately 20% of maximal occupancy) to high density (approximately 100% of maximal occupancy) and any amount in between (i.e., 30, 40, 50, 60, 70, 80, or 90 percent maximal occupancy), or at a density of less than 500, 300, 200, 100, 80, 50, 40, 20 or 10 data oligonucleotides per 100 nm². Applicant has discovered that increasing the percentage of data oligonucleotide occupancy is correlated with increased aggregation of the DNAFiles. However high occupancy can still be achieved with minimal aggregation if the data oligonucleotides are attached to only one surface of a multi-sheet conformation of the DNAFiles. In one embodiment the data oligonucleotides are uniformly distributed over only one surface of the DNAFile.

In accordance with one embodiment the DNAFiles each have the shape of a multi-layered sheet, optionally a rectangular or square sheet, having only the top surface populated with data oligonucleotides at 40, 60, 80 or 100% occupancy. In one embodiment modifications are made to stabilize the DNAFiles sheet shape as a planar shape (i.e. holding the multi-layered sheet conformation in a more of a two-dimensional shape than a twisted three-dimensional), and these modifications include one or more of the following:
a) adding a sequence of six or more thymidine resides (poly(T)) to the end of the noncomplementary sequence of the data oligonucleotides;
b) decreasing staple length around sheet corners to less than 100 nucleotides, or less than 50 nucleotides, to allow for flexibility during folding process;
c) adding additional crossover staples that bind to noncontiguous sequences of the DAN scaffold to improve stability and shape of the origami folded construct;
d) introducing intentional gaps or missing base pairs within the scaffold DNA strand/staple folded structure (i.e. “skips”) near the center-line of the folded multi-layered sheet to decrease twist.

In one embodiment the data oligonucleotides share base pair complementarity with the DNA scaffold but do not participate in the folding of the DNA scaffold. Such single stranded DNA non-staple oligonucleotides comprise a region complementary to said DNA scaffold and a region non-complementary to said DNA scaffold, wherein the non-complementary region comprises nucleic acid sequences that encodes digital information, optionally wherein the non-complementary region of the non-staple oligonucleotides further comprises primer binding sequence and optionally bar coding sequences.

In one embodiment a method of retrieving digital data stored in DNA is provided. The method comprises providing a library of origami folded DNA files (DNAFile), wherein each DNAFile comprises a single stranded scaffold DNA, a plurality of staple oligonucleotides and a plurality of data oligonucleotides, with a unique set of data stored within the sequences of the scaffold DNA and/or staple oligonucleotides of the DNAFiles. In one embodiment the data is stored only in the noncomplementary sequence of the data oligonucleotides. The library of DNAFiles is subjected to denaturing conditions to at least partially disrupt the hybridized duplex between the single stranded staple oligonucleotides and the DNA scaffold and between the single stranded data oligonucleotides and the DAN scaffold, followed by PCR amplification of the nucleic acid sequences containing the primer binding sequences to produce amplicons. The staple oligonucleotides and data oligonucleotides are then reannealed with the DNA scaffold to reconstitute the folded origami DNAFiles and the synthesized amplicons are separated the from the reconstituted folded origami DNAFiles. The separated reconstituted folded origami DNA file(s) (DNAFiles) are then returned to storage and the separated and recovered amplicons are sequenced to retrieve digital data encoded by the DNAFile. In accordance with one embodiment individual DNAFiles are selected from the original library, and only the selected DNAFiles are subject to the denaturing and amplification steps, wherein the reconstituted folded origami DNA file(s) (DNAFiles) are returned to the non-selected members of the original library to reconstitute the original full library, prior to returning the reconstituted library to storage.

The library of DNAFiles can be stored in ambient temperatures in a lyophilized state. Other means of stabilizing DNA origami structures are known those skilled in the art.

### BRIEF DESCRIPTION OF DRAWINGS

FIG. 1 is a schematic drawing showing how DNA origami leverages the complementary base pairing property of DNA to “fold” a large single stranded “scaffold” DNA, with the help of a plurality of short oligonucleotide “staples”, into pre designed two or three dimensional structures. Using this strategy it is possible to pack several thousand bases (wherein the bases are selected to code for bits) into a nanostructure having nanometer dimensions. Advantageously, using DNA origami structures allows data to be directly encoded into the nucleotide bases of the staple oligonucleotides, data oligonucleotides, and/or scaffold DNA (providing high density compaction of the data).

FIG. 2 is a photograph of a gel comparing the electrophoretic mobility of origami folded sheets that differ from each other based solely on differing combinations of data oligonucleotide occupancy. More particularly, lane 1 represents a set of molecular markers, lane 2 represents the folded scaffold absent any data oligonucleotides, lanes 3-7 represent folded scaffolds with both sides of the scaffold populated with data oligonucleotides at 20%, 40%, 60%, and 100% occupancy, respectively, and lanes 8-9 represent folded scaffolds with only one side of the scaffold populated with data oligonucleotides at 20% and 100% occupancy, respectively. The data demonstrate that increased density of data oligonucleotides on the DNA scaffold results in greater aggregation. However, populating data oligonucleotides only on the top surface of an origami DNA scaffold folded into a sheet conformation greatly diminishes the formation of aggregates.

FIGS. 3A and 3B are representations of origami DNA folded into the sheet conformation. FIG. 3A is a schematic drawing of origami folded sheet configuration, having a single folded origami layer, a double layered sheet where the two sheet are in an anti-parallel relationship, and a double layered sheet where the two sheet are in an anti-parallel relationship. FIG. 3B is a computer generated modeling of the origami folded sheet configuration in the absence of stabilizing modifications and with stabilizing modifications (introducing intentional gaps or missing base pairs within the scaffold DNA strand/staple folded structure near the center-line of the folded bilayer sheet).

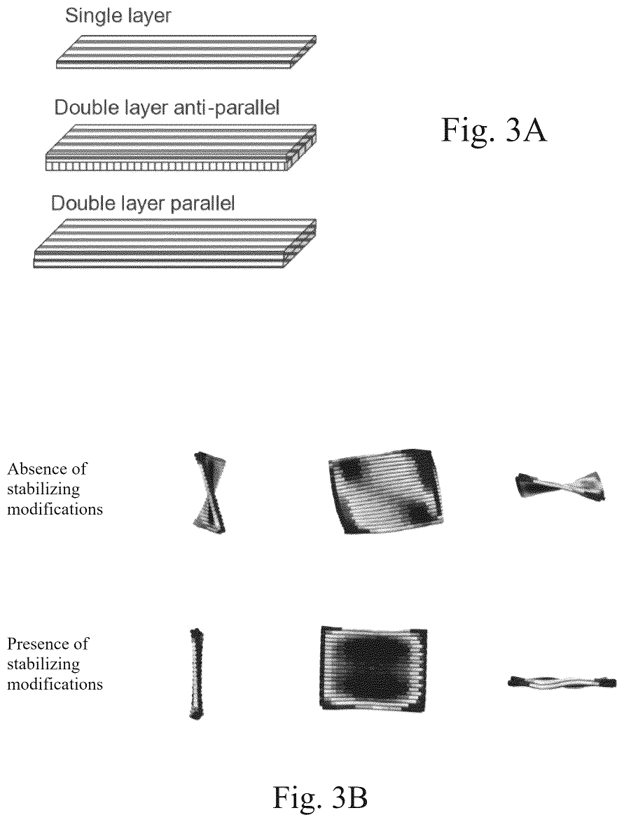

FIGS. 4A and 4B provide schematic representations of a DNAFile. The exemplified DNAFile comprises two single stranded scaffold DNA sequences (“0” and “1”) joined to one other by staple oligonucleotides, wherein the staple oligonucleotides comprise a nucleic acid sequence that is complementary to a sequence on scaffold DNA strand “0” and a sequence that is complementary to sequence on strand “1”. The DNAFile is further provided with data oligonucleotides that have complementarity with a sequence of scaffold DNA sequence “0”. The data oligonucleotides comprise four components: a sequence that shares complementarity with the single stranded scaffold DNA, a sequence that encodes digital information, and a pair of primer binding sequence that flank the sequence that encodes digital information. FIG. 4A provides an example wherein the length of the sequence encoding digital information can be varied while retaining an overall length of about 80 to 100 nucleotides. In this embodiment the sequence that shares complementarity with the single stranded scaffold DNA is located at one end of the data oligonucleotide. In FIG. 4B, the data oligonucleotide has two noncomplementary overhangs, wherein a first primer binding sequence is located at one end of the data oligonucleotide and a second primer binding sequence is located at the other end of the data oligonucleotide with the sequence that shares complementarity with the single stranded scaffold DNA and the data encoding sequence being located between the first and second primer binding sequences.

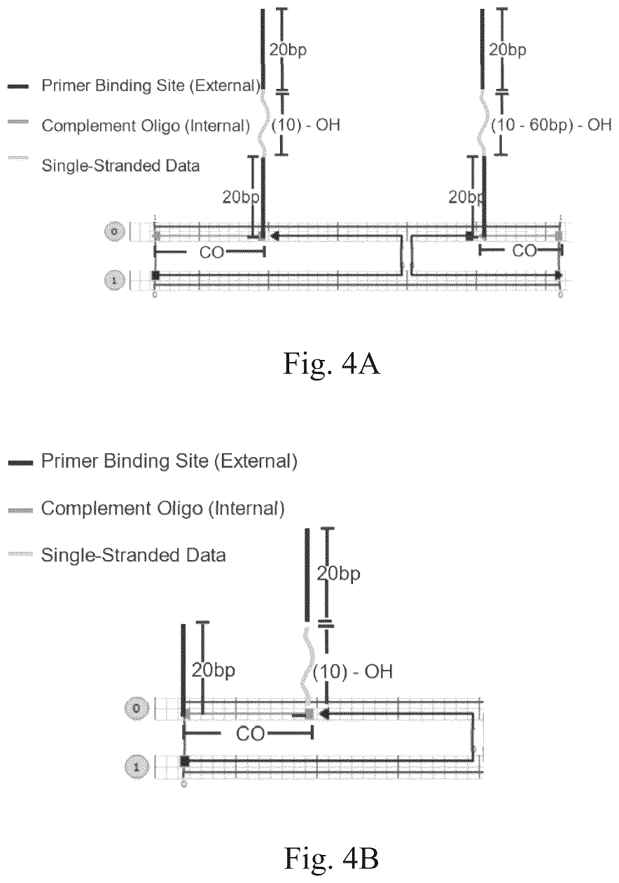

### DETAILED DESCRIPTION

### Definitions

As used herein, the term “complementary base pairing” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.” As used herein the term “complementarity” when used in the context of a nucleic acid sequence, defines a level of sequence identity between two nucleic acid sequences that allows for specific hybridization between the two respective sequences.

As used herein the term “DNA scaffold” defines a large single stranded DNA of approximately 500 to about 31,000 bases which is folded by a plurality of preselected complementary DNA staple oligonucleotides.

As used herein the term “single stranded DNA staple oligonucleotide” or “staple oligonucleotide” defines a nucleic acid sequence that will self-assemble with a single stranded DNA scaffold to reversibly fold the single stranded DNA scaffold into a compacted 2-D and 3-D structure. Staple oligonucleotides typically comprise two or more nucleic acid sequences that are complementary, optionally having at least 80%, 90%, 95% or 99% sequence identity, to non-contiguous sequences present in a DNA scaffold, wherein the staple oligonucleotide sequences sharing complementarity with the DNA scaffold are linked to one another via a linking nucleic sequence, optionally wherein the linking nucleic acid sequence that lacks complementarity with the DNA scaffold.

As used herein the term “data oligonucleotide” defines a nucleic acid sequence comprising a sequence sharing at least 80%, 90%, 95% or 99% sequence identity with a corresponding scaffold DNA sequence, a data encoding sequence, and a first and second primer binding sequence flanking the data encoding sequence, i.e., where the first primer binding sequence is 5′ to the data encoding sequence and the second primer binding sequence is 3′ to the data encoding sequence.

As used herein the term “DNAFile” defines an origami folded construct comprising a single stranded DNA scaffold that is hybridized to a plurality of smaller DNA staple oligonucleotides, and a plurality of data oligonucleotides, wherein the hybridization of the plurality of staple oligonucleotides to the single stranded DNA scaffold cause the DNA scaffold to fold into a three dimensional shape, wherein the shape is reversible upon dissociation of the staples with the scaffold DNA.

As used herein the phrase “nucleic acid sequences that encodes digital information” or “data encoding sequence” defines a synthetic nucleic acid sequence wherein the sequence of the nucleotides has been selected to represent binary data. Several methods for encoding text are known to those skilled in the art. Most of these involve translating each letter into a corresponding “codon”, consisting of a unique small sequence of nucleotides in a lookup table. Some examples of these encoding schemes include Huffman codes, comma codes, and alternating codes (see Smith G C, Fiddes C C, Hawkins J P, Cox J P (July 2003). “Some possible codes for encrypting data in DNA”. Biotechnology Letters. 25 (14): 1125-1130).

### EMBODIMENTS

The present disclosure is directed to compositions and methods for overcoming the challenges associated with archival and random access of data stored in DNA. More particularly, the present disclosure describes the use of DNA Origami (DNAO) techniques to package and retrieve data encoding DNA strands. DNA origami structures are described in U.S. Pat. No. 9,765,341, the disclosure of which is incorporated herein by reference. In the present approach libraries of DNAO structures (DNAFiles) are provided that will act both as a filing and addressing system for storing data encoding DNA molecules and will allow for a straightforward single step method for random access without the need for permanently removing the nucleic acids encoding the data from the storage pool.

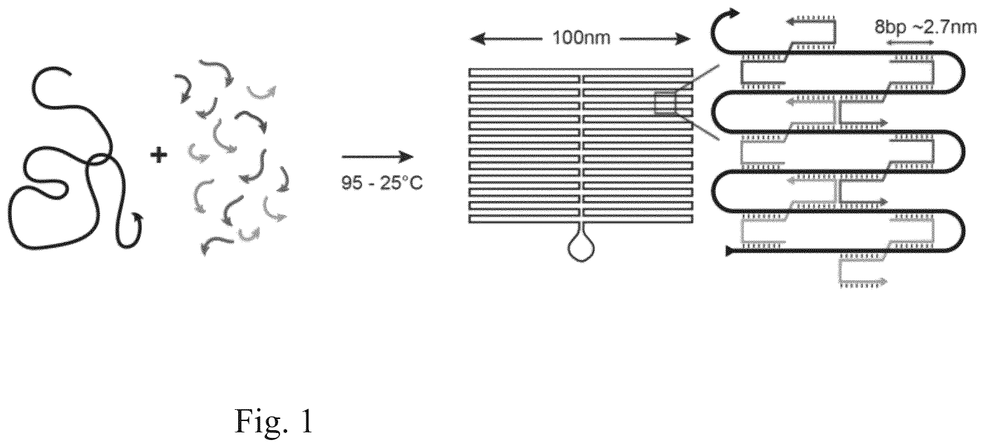

In accordance with one embodiment compositions and methods are provided for packaging and archiving data stored in indexed DNA into individual DNAFiles. DNAFiles, in addition to providing organization and compartmentalization, offer the unique advantage of PCR retrieval of data without loss in organization or material consumption. Current strategies that employ PCR based data retrieval rely on additional steps to physically separate subsets from a complex pool before amplification which increase system complexity and lead to an irreversible loss of the retrieved data. The approach disclosed herein provides a single-step approach to enable reversible, high-fidelity multiplexed PCR by creating a library of physically isolated files that can be retrieved on demand. In one embodiment of this method, information is encoded and written in indexed DNA oligonucleotides (data oligonucleotides), and the data oligonucleotides in combination with staple oligonucleotides are mixed with scaffold DNA and folded via thermal annealing using DNA origami techniques (see FIG. 1) and stored as libraries of DNAFiles.

Random access via PCR amplification provides data retrieval upon denaturing the DNAFiles, and data restoration is accomplished via re-annealing of the denatured DNAFiles to reconstitute the original library of DNAFile. More particularly, in one embodiment retrieval of digital data stored in DNA is achieved by obtaining one or more DNAFiles of a library of origami folded DNAFiles and at least partially separating the single chain scaffold from the staple to allow PCR amplification of the staple oligonucleotides. In one embodiment only a subset of the bound staple oligonucleotides and data oligonucleotides are released from the single chain scaffold DNA. There is a large toolbox of methods to selectively open and close specific DNAO structures to access only a sub selection of data bearing oligonucleotides. For example, the use of “toe-holds”, where staple oligonucleotides are displaced by addition of other oligonucleotides with higher affinity, or the use of changes in Ionic strength or pH, enzymatic or UV cleavage techniques can be used to selectively open and close specific DNAO structures. Combining 2 or more of these features can provide a wide array of strategies for random access of small subsets of DNAO based data files from a pool of many DNAO files. Switchable actuation in DNA Origami allows any DNAFile to be selectively opened for reading and then closed again for storage.

Once a DNAFile has been unfolded, by denaturing a folded origami DNAFile of the library to release the single stranded staple oligonucleotides and the data oligonucleotides from the DNA scaffold, PCR amplification can be conducted on select nucleic acid sequences of said denatured DNA scaffold, the data oligonucleotides and staple oligonucleotides to produce amplicons, wherein the amplicons comprise the encoded data. Once the amplification step is completed, the original DNAFiles are reconstituted by altering the conditions to allow the staple oligonucleotides to reanneal to the single strand scaffold DNA to refold the scaffold DNA and reconstitute the original DNAFile. The remaining amplicons can then be analyzed to retrieve digital data encoded by the DNAFile. Advantageously, the reconstituted DNAFiles can be used to confirm the accuracy of the amplification step. Failure to faithfully copy the template during the PCR amplification will result in a failure to reconstitute the DNAFile as detected by an alteration in the shape or size of the DNAFile. Once the reconstituted DNAFiles have been confirmed as having the correct size and shape, they are returned to the library from which they were isolated. The PCR produced amplicons are separated from the reconstituted folded origami DNAFile and sequenced to retrieve digital data encoded by the DNAFile.

In one embodiment the digital data is only located within the sequences of the data oligonucleotides of the DNAFiles. Upon release of the single stranded data oligonucleotides from the DNA scaffold, the data encoding sequence of the data oligonucleotides can be amplified by standard PCR methods using PCR primers that specifically bind to the first and second primer binding sequences that are located on either side of the data encoding sequence. In accordance with one embodiment the first and second primer binding sequences are located at the 5′ terminus and 3′ terminus, respectively, of the data oligonucleotide, so PCR amplification produces an amplicon comprising the entire data oligonucleotide, including the data encoding sequence and the sequence having at least 80%, 85%, 90%, 95% or 99% sequence identity with a sequence located in the DNA scaffold. In one embodiment the nucleic acid sequences of the staple oligonucleotides and the data oligonucleotides that have complementarity with the DNA scaffold share 100% sequence identity with the corresponding DNA scaffold DNA nucleic acid sequences.

In one embodiment the reconstituted DNAFiles and PCR produced oligonucleotide amplicons are separated by electrophoresis or other techniques such as size exclusion chromatography or affinity binding, after which the reconstituted DNAFiles are returned to information storage and the generated oligonucleotide amplicons are read via sequencing to retrieve the data stored on the data oligonucleotides.

In one embodiment, the single strand scaffold DNA has a size of at least 500 bases, and more particularly a size selected from the range of about 500 bases to about 31 kb, about 1 kB to about 25 kb or about 2 kB to about 15 kb. In one embodiment the single strand scaffold DNA has a size selected from group consisting of 0.5 kB, 1 kB, 2 kB, 4 kB, 5 kB, 8 kB, 10 KB, 15 kB, 20 kB, and 25 kB. In one embodiment each DNAFile of the library disclosed herein comprises 100 to 300 staple oligonucleotides that each comprise distinct nucleic acid sequences that share complementarity with 2, 3, 4 or more corresponding non-contiguous nucleic acid sequences present in the single strand scaffold DNA of the DNAFile. The individual staple oligonucleotides of each DNAFile can vary in length and have sizes independently selected from a range of about 25 to about 200 nucleotides.

Data oligonucleotides have sizes independently selected from a range of about 50 to about 200 nucleotides, wherein the first and second primer binding sequences range in size from about 10 to about 20 nucleotides, the nucleic acid sequence having complementarity with its corresponding DNA scaffold sequence ranging in size from about 10 to 20 nucleotides, and the data encoding sequence comprising the remaining nucleotides of the data oligonucleotide (i.e., ranging from about 10 nucleotide to about 170 nucleotides). In one embodiment the data oligonucleotides comprise a sequence noncomplementary to the scaffold DNA (i.e., an overhang region) of at least 50 nucleotides, optionally ranging from about 60 to about 180 nucleotides.

Each library represents a mixture of large number of individual DNAFiles, including for example 103, 104,105, 106 or more individual DNAFiles. The libraries can be stored using standard techniques to enhance the stability of nucleic acids including maintaining DNA origami structure integrity. In one embodiment the libraries can be stored in aqueous form by freezing the samples and maintaining the samples in ultra-low freezers, typically at or below −80° C. or in liquid nitrogen. Cryoprotectants can be added to protect DNA Origami structure for up to 1000 freeze/thaw cycles. See Xin, Y. et al. Cryopreservation of DNA Origami Nanostructures. Small, vol. 16 (13) (2020). In addition encapsulation in polymer or organosilica structures can provide increased stability. Koch, J. et al. Preserving DNA in Biodegradable Organosilica Encapsulates. Langmuir 38, 11191 11198 (2022).

In one embodiment the DNA libraries are stored in a dry form. For example lyophilization at ambient temperatures keeps DNAO intact after being treated to a 10 day accelerated aging test, equivalent to ˜100 days at room temperature. In one embodiment the DNA libraries are stored by desiccation in the presence of an adjuvant such as polyvinyl alcohol (PVA) or the disaccharide sugar trehalose, present at a final concentration of around 1.5 percent.

In accordance with one embodiment the individual DNAFiles are each provided with their own unique bar coding sequences that allow for the selection of a single DNAFile. Alternatively, subsets of DNAFiles from all the DNAFiles present in a particular library can be provided with different unique barcoding sequences to allow the selection of one or more preselected subgroups of DNAFiles from all the DNAFiles present in a particular library. DNA barcodes are linked to moieties (e.g., nucleic acid sequences) that are capable of binding to the surface of each DNAFile while presenting the bar code for interaction with other moieties. In one embodiment, the barcode is a unique nucleic acid sequence relative to other the nucleic acid sequences of the DNAFile, wherein said nucleic acid sequence further comprises a sequence having complementarity (optionally having at least 90%, 95%, or 99% sequence identity, or 100% sequence identity) with a nucleic acid sequence of the DNA scaffold of a DNAFile.

In one embodiment, a library of DNAFiles is provided wherein each DNAFile, or certain subsets of DNAFiles of the library, are provided with their own unique nucleic acid barcode construct. In embodiments where the DNAFiles are barcoded, the nucleic acid barcode construct can be associated with the DNAFile via base-pairing. In this embodiment, the base-pairing can occur between a sequence of a single-stranded overhang on the DNAFile and a complementary sequence appended to the nucleic acid barcode construct.

In other embodiments, the nucleic acid barcode construct can be associated with the DNAFiles by a high affinity, non-covalent bond interaction between a biotin molecule on the 5′ and/or the 3′ end of the nucleic acid barcode construct and a molecule that binds to biotin present on the DNAFile. In this embodiment, the molecule that binds to biotin can be bound to the DNAFile by a covalent phosphoramidate bond formed via an EDC-NHS coupling reaction between a terminal phosphate group of a 5′ end of an overhang on the DNAFile and an amine group on the molecule that binds to biotin. In this embodiment, the biotin can be bound to the nucleic acid barcode construct by a covalent bond.

In one illustrative embodiment, the nucleic acid barcode construct can be bound to the DNAFile by a covalent bond. In this embodiment, the covalent bond can be formed via an EDC-NHS coupling reaction between a terminal phosphate group of the 5′ end of an overhang on the DNAFile and an amine group on an amino terminal nucleotide of the nucleic acid barcode construct. In another embodiment, the covalent bond can be formed via a click chemistry coupling reaction between an azide group on the DNAFile and an alkyne group on the nucleic acid barcode construct. In yet another embodiment, the covalent bond can be formed via a click chemistry coupling reaction between an azide group on the nucleic acid barcode construct and an alkyne group on the DNAFile. In still another embodiment, the nucleic acid barcode construct can be associated with the DNAFile by a covalent bond between a carboxy terminated molecule on the DNAFile and a primary amine on the nucleic acid barcode construct at the 5′ and/or the 3′ end.

In one aspect, the nucleic acid barcode construct can comprise a polynucleotide barcode and the barcode comprises a unique sequence not present in any known genome for identification of the polynucleotide barcode. In another embodiment, a set of different nucleic acid barcode constructs with different polynucleotide barcodes (e.g., 88 or 96 different polynucleotide barcodes) can be used to allow for multiplexing of multiple data bearing oligonucleotides on one sequencing run of a DNAFile, wherein subsets of staple oligonucleotides of a given DNAFile are associated with distinct barcodes.

In various embodiments, the barcodes can be from about 5 to about 100 bases in length, from about 5 to about 90 bases in length, from about 5 to about 80 bases in length, from about 5 to about 70 bases in length, from about 5 to about 60 bases in length, from about 5 to about 50 bases in length, from about 5 to about 40 bases in length, from about 5 to about 35 bases in length, about 5 to about 34 bases in length, about 5 to about 33 bases in length, about 5 to about 32 bases in length, about 5 to about 31 bases in length, about 5 to about 30 bases in length, about 5 to about 29 bases in length, about 5 to about 28 bases in length, about 5 to about 27 bases in length, about 5 to about 26 bases in length, about 5 to about 25 bases in length, about 5 to about 24 bases in length, about 5 to about 23 bases in length, about 5 to about 22 bases in length, about 5 to about 21 bases in length, about 5 to about 20 bases in length, about 5 to about 19 bases in length, about 5 to about 18 bases in length, about 5 to about 17 bases in length, about 5 to about 16 bases in length, about 5 to about 15 bases in length, about 5 to 14 bases in length, about 5 to 13 bases in length, about 5 to 12 bases in length, about 5 to 11 bases in length, about 5 to 10 bases in length, about 5 to 9 bases in length, about 5 to 8 bases in length, about 6 to 10 bases in length, about 7 to 10 bases in length, about 8 to 10 bases in length, or about 6 to about 20 bases in length.

In accordance with one embodiment, individual DNAFiles are barcoded and individual DNAFiles or subsets of DNAFiles of the library can be selected and separated from other DNAFiles of the library by selectively binding the desired DNAFiles to a complementary oligonucleotide immobilized on a surface, or oligonucleotides bound to magnetic or fluorescently labelled nanoparticle. This step allows for retrieval of data from a targeted subset of a library of data storing DNAs while leaving the remaining members of the library unperturbed. In another embodiment subsets of staple oligonucleotides of a single DNAFile can be provided with different primer binding sequences to allow for data retrieval from a select group of staple oligonucleotides of a DNAFile selected from the library of DNAFiles by barcoding.

Various embodiments of barcodes are shown below in Table 1 (labeled “Polynucleotide Barcodes”). These barcodes can be used in the nucleic acid barcode constructs alone or in combinations of, for example, two or more barcodes, three or more barcodes, four or more barcodes, etc. In the embodiment where more than one barcode is used, the hamming distance between the barcodes can be about 2 to about 6 nucleotides, or any suitable number of nucleotides can form a hamming distance, or no nucleotides are present between the polynucleotide barcodes.

TABLE 1

| | SEQ |
|---|---|
| Polynucleotide | ID |
| Barcodes | NO: |
| GCTACATAAT | 1 |
| ATGTTACACA | 2 |
| TGGGGCCCAA | 3 |
| TAGTTTATCC | 4 |
| ACCCCGTCTT | 5 |
| CCGGCCATCA | 6 |
| GAGCTTGCTC | 7 |
| ACGTTCTATA | 8 |
| TACAGCAAAA | 9 |
| GTTAGGTGGT | 10 |
| GGAGACCGAC | 11 |
| TGGCCCCTTG | 12 |
| TGGCCGTAAG | 13 |
| CGTTCGTCAA | 14 |
| CGGACGTGGA | 15 |
| AGAGGGGGCA | 16 |
| GTTCAGGTCG | 17 |
| CTCGCAAGAG | 18 |
| GCAACGACTT | 19 |
| GCCATCCATC | 20 |
| TTCCGAGCAG | 21 |
| CTTCTGGACA | 22 |
| AACATTAGAC | 23 |
| AAGCAATAGT | 24 |
| AGGGTAAGAC | 25 |
| CGTTGTCTTG | 26 |
| TTTCCCCGCC | 27 |
| CGAATGGATC | 28 |
| CATCACTTGC | 29 |
| CTCTCGCACT | 30 |
| GTTCACGTGC | 31 |
| AATAAGCCTG | 32 |
| GTTAACAATT | 33 |
| ATTCAGATCC | 34 |
| CCTGCTGATT | 35 |
| CTTGGTCATA | 36 |
| TCTTCCTGTT | 37 |
| ACTGCCATGG | 38 |
| CATGTATAGT | 39 |
| GGTAGCGGCA | 40 |
| TCACTCTAAC | 41 |
| AAGGTGCACC | 42 |
| AATGCTCGTT | 43 |
| TGTCTAGAAA | 44 |
| CTGCCTGCCT | 45 |
| ACTATAAAAG | 46 |
| TAGTATCGAG | 47 |
| ATCGCAGTCC | 48 |
| TCATCAGAAC | 49 |
| TCCTAGACGC | 50 |
| GCCGGGCGGG | 51 |
| GCCCAGAAGA | 52 |
| CTTAGAGCTG | 53 |
| GTCTGCGCTT | 54 |
| CGCCGTCCTT | 55 |
| TTTATCTGCT | 56 |
| TGCTTCGGAG | 57 |
| GGGGAGAATG | 58 |
| GTGGTAAGTG | 59 |
| GAAATTAGTA | 60 |
| GCTATCCTAA | 61 |
| ATCTGTACGA | 62 |
| AGTTCGGGGC | 63 |
| CGAGTCTGTC | 64 |
| ATCCTACGCA | 65 |
| ATGGTGGATA | 66 |
| CCTCTAACTA | 67 |
| ATAGCTGCAC | 68 |
| GACAGAATTT | 69 |
| CAATTGGCAT | 70 |
| TCTAGTAGAC | 71 |
| TTATTCATGG | 72 |
| TTGGCAACCG | 73 |
| CATAATACAT | 74 |
| ACAGACTCAC | 75 |
| GCGATGCTGC | 76 |
| CATCTTTGCC | 77 |
| GTGACTCCAG | 78 |
| GGACGAGTCT | 79 |
| TAGTGGCGTG | 80 |
| AACGCAGCTT | 81 |
| AGAACAGGTG | 82 |
| AGGCTATGTT | 83 |
| CCTGGATCTT | 84 |
| CTAGCCGGCC | 85 |
| ACCAGTTATC | 86 |
| ACGTTATAGC | 87 |
| TCGAGTTTGA | 88 |
| TGAAGCGAGC | 89 |
| GACTGGCGAA | 90 |
| GATGGACCTA | 91 |
| GTCCACAACG | 92 |
| CCTCCCCAGA | 93 |
| TTATGACGCC | 94 |
| CTTGATCCGT | 95 |
| AATGCGCAAT | 96 |
| GTACCCCTCA | 97 |
| CGACAGCTCG | 98 |
| TGACCTGGCT | 99 |
| TTCATAGCCC | 100 |
| CCCAAGAGAA | 101 |
| AAACGAAGTA | 102 |
| GACGTTTACA | 103 |
| GATCGATTTG | 104 |
| CACTGTCACC | 105 |
| TGTGAGAGTT | 106 |
| GACGTAACCT | 107 |
| CAGACTCTGC | 108 |
| TATGCCAATA | 109 |
| ACAGGTGATG | 110 |
| GTCATCGCGT | 111 |
| TCTTATAAAC | 112 |
| GTGTAGACTG | 113 |
| AAACAACCGG | 114 |
| ATCCTGTACC | 115 |
| TTATAAGAAT | 116 |
| ATAAGTAGGC | 117 |
| TCTCGTAAGG | 118 |
| GATCCGCCGC | 119 |
| TGTCAGGTTT | 120 |
| TCCGAAGCCC | 121 |
| TCCATGTCCA | 122 |
| GTGATGGTAC | 123 |
| CTCCACATAC | 124 |
| TTCGGATGAG | 125 |
| ACGACATCGC | 126 |
| GAGATGCACA | 127 |
| TTTGTATGGC | 128 |
| CTTTTCTAGA | 129 |
| AGTCTAATCA | 130 |
| GACTTAGCCA | 131 |
| TATCACAGTA | 132 |
| AAGCTCGAGT | 133 |
| TGTTACGACA | 134 |
| AAGGATAGTC | 135 |
| GCACTTAGCC | 136 |
| GAGGGATCCG | 137 |
| ATTCTAGAAG | 138 |
| GATAACTGAT | 139 |
| ATCTGACTGT | 140 |
| CAAAGCGAAC | 141 |
| GAAATTGCGA | 142 |
| GGGTCCAGTC | 143 |
| ATCAGGTAGC | 144 |
| GAAAGGTCCT | 145 |
| GGCTACCACA | 146 |
| TTATTGCTGA | 147 |
| CGCCGCGTTT | 148 |
| TTTTCAAAAG | 149 |
| CTGGGCTAAA | 150 |
| CCCGATGAGA | 151 |
| TGGGAAATAT | 152 |
| GTACGAGCGG | 153 |
| GCGTGCAGCT | 154 |
| AGTCTGCGGA | 155 |
| TAACTATTTA | 156 |
| GAGTTGCCGG | 157 |
| CAGCCCGGCG | 158 |
| TCACCTACAT | 159 |
| AGTGGCTAAC | 160 |
| AGAATGTGAG | 161 |
| TAGTTTCGCA | 162 |
| CTTCATTTCT | 163 |
| GCCATGATAT | 164 |
| ACGGCAAATC | 165 |
| ATCGATAGTA | 166 |
| CCTAAAGGCA | 167 |
| TACGAGCGGT | 168 |
| TTTGTCGTCG | 169 |
| TACAAGCTTG | 170 |
| GACCAACACG | 171 |
| GAACGACGAA | 172 |
| TCGGAACGCA | 173 |
| ATCCGGTGGT | 174 |
| TAAAACGTAG | 175 |
| TATGTGAGCC | 176 |
| GAGGCATCGA | 177 |
| GAATGGGTGG | 178 |
| AACGACACAA | 179 |
| GTACGATGCA | 180 |
| AGAAGGCGCC | 181 |
| CCGCAATGGA | 182 |
| TACGGATTTT | 183 |
| GTCGTTAGCT | 184 |
| GGACTAGGGC | 185 |
| ATTGGTATTC | 186 |
| ATCCCAGAGA | 187 |
| GTCCCAGCTC | 188 |
| CACGAGGAAT | 189 |
| TACAATTGCA | 190 |
| ATTCCTGAAT | 191 |
| TAGCGAGGCG | 192 |
| CTGGATGGGC | 193 |
| GCGACGGCCA | 194 |
| ACCTGCACAA | 195 |
| CATGACAGAC | 196 |
| TTACCAACGT | 197 |
| CAGGTGTGTG | 198 |
| CGAGGGACGG | 199 |
| CGTCTCGGTA | 200 |
| TAAGCTATCT | 201 |
| TACTCCCCTA | 202 |
| TTATATTCAT | 203 |
| AGCGATCTGC | 204 |
| TCTTCTGATC | 205 |
| ATAGTTCCCA | 206 |
| TTTACGGGTG | 207 |
| GTGTCCCCTG | 208 |
| GCGGGGGTCG | 209 |
| CATTGATCTA | 210 |
| AGGGACGGTG | 211 |
| CAGTTACTTT | 212 |
| CCATACTTCC | 213 |
| ATCAGAATTA | 214 |
| AAACTAGGCA | 215 |
| AATGTCGTTG | 216 |
| CACATGGGTC | 217 |
| GGTCGCTGGT | 218 |
| ACTGTATTAC | 219 |
| CCGAGACGCG | 220 |
| ACTCCAACCC | 221 |
| ATATTACAAG | 222 |
| CCATGGATAG | 223 |
| CCGTCTCAAT | 224 |
| GATCGTCGGG | 225 |
| TCTTGTTTTG | 226 |
| AATATTGCTC | 227 |
| AACGTCGTCT | 228 |
| AATATTTTTG | 229 |
| CGTAACGTGC | 230 |
| GCGTGGTTAT | 231 |
| CAAAACATTA | 232 |
| CGTATCCTGA | 233 |
| TCGCTTACAA | 234 |
| TCCATTGTGT | 235 |
| GCCCCCATTC | 236 |
| TGACGTCTAT | 237 |
| TGGGCCGAGG | 238 |
| AAGTGTCAAG | 239 |
| GACAGTAGAG | 240 |
| CGCAGCCATC | 241 |
| GAGGCAGAAC | 242 |
| GTTGAAATTG | 243 |
| ATCTGATAAA | 244 |
| AGCTGTCTCT | 245 |
| TTTTAGGTTA | 246 |
| TATCTGTCCG | 247 |
| AAAACATATG | 248 |
| GTAAAGAAGA | 249 |
| TCGACGTGCA | 250 |
| TAGATCTTAA | 251 |
| CACTGGTCAC | 252 |
| ATTCTGATGT | 253 |
| ATGGCCCTGA | 254 |
| GGTGATGAGA | 255 |
| CACCGTGGGG | 256 |
| GCTTGCTCGG | 257 |
| CCAGTTGAAC | 258 |
| CGTCTGTACC | 259 |
| CCAACGCGGC | 260 |
| ACGTGATCGA | 261 |
| CCATCGAATC | 262 |
| CGGTGTCTGC | 263 |
| AAACCACCTC | 264 |
| TCAATGTTCC | 265 |
| TTCGACATGT | 266 |
| AGGCACGATA | 267 |
| CACGAGATCA | 268 |
| CATGCTGGGG | 269 |
| TACCATGGTT | 270 |
| TTGCCCATAT | 271 |
| TGCACATTCG | 272 |
| GTTATGTTGG | 273 |
| TGAGTTATGA | 274 |
| GATGGCCCCC | 275 |
| GATGGGTTAC | 276 |
| AGCTACGTTG | 277 |
| ACCCCATGCA | 278 |
| TACTACCGTT | 279 |
| TCGCTTCTAC | 280 |
| CTGGCAGTGC | 281 |
| TCTATATATA | 282 |
| GGATTAGTTC | 283 |
| GTGTTACGCT | 284 |
| TCGACTCCGT | 285 |
| GGTAGCAGGC | 286 |
| TATTGGATTC | 287 |
| GTTCGATCGA | 288 |
| ATATTAATAT | 289 |
| AGAACGATTG | 290 |
| GTAAAGTGTA | 291 |
| CCCATGTGCC | 292 |
| GTGGCCTCGC | 293 |
| GACACTAGGA | 294 |
| ATATTCTGAC | 295 |
| TAAGTAGACG | 296 |
| TAACGGTCTA | 297 |
| TAGTTTCATT | 298 |
| TTGGATCCGA | 299 |
| CGTGACAACC | 300 |
| CGCGCTCAGA | 301 |
| CGTTCTTAAT | 302 |
| ACAAGAGTTT | 303 |
| AGGGTTATAG | 304 |
| ACCACGACTC | 305 |
| GTACTCGGGG | 306 |
| ACAAATATCT | 307 |
| GATCGGGGTG | 308 |
| ATGTAACTCC | 309 |
| ATGAAGAAGC | 310 |
| ATGTATTGTC | 311 |
| TGCATTGGAA | 312 |
| GCGGACGATC | 313 |
| CCGTACTTGA | 314 |
| TTTGCCCCCG | 315 |
| ACCTCACGCG | 316 |
| ATTAAGGGGC | 317 |
| CGTGGACATG | 318 |
| TTAGCCCTTC | 319 |
| CGAGAGTTTG | 320 |
| TGCATCCTCT | 321 |
| TGCGATTCCG | 322 |
| TTATTACGTT | 323 |
| TGATGTGGTT | 324 |
| GGGCGTCAAT | 325 |
| CCCTTGAAAT | 326 |
| TCTTTGGGGC | 327 |
| ACCGGCAGGC | 328 |
| GCTAAAATCT | 329 |
| GCCGTTGACG | 330 |
| GGAGTTGTTG | 331 |
| TACTTGAGAA | 332 |
| CGGGTGCGCT | 333 |
| AAAAGCGTCT | 334 |
| GTAAAGATAG | 335 |
| GCCTGGTCAG | 336 |
| GGCAAAAAGG | 337 |
| ACCCTTCTCT | 338 |
| TCACATAGTG | 339 |
| TCGTCTGTGC | 340 |
| TGCTCGGATC | 341 |
| AGCAGTCCCG | 342 |
| TTTGGGCTGT | 343 |
| CTCACGATCT | 344 |
| TGGCGCATAC | 345 |
| GCAATTGAAA | 346 |
| TCGGGAGACG | 347 |
| CCCGGCGAAA | 348 |
| TGATGCGGAA | 349 |
| AACTGAGGCG | 350 |
| CATATTATTT | 351 |
| AAAAGTCATT | 352 |
| AAGCGGTGAG | 353 |
| AAGGTAATCA | 354 |
| CTGACACTTA | 355 |
| CTGTTTTCTA | 356 |
| CACATGGCAG | 357 |
| TTCAATCCGG | 358 |
| TGTCCGGCAT | 359 |
| TGGTACCGTG | 360 |
| AAGAGATATT | 361 |
| GATGTACTAC | 362 |
| GAAATGGAAT | 363 |
| TTAAAATACT | 364 |
| TGACCGGAAC | 365 |
| GTCGCCGCAA | 366 |
| TAGGATACCG | 367 |
| AGTCCAATTG | 368 |
| GGGGGCTATA | 369 |
| ACCTTCAGTT | 370 |
| ATGGCAAGTA | 371 |
| AGAATGTTTT | 372 |
| AGTTCGTTTG | 373 |
| CACTACTGAC | 374 |
| GATCAAGAGC | 375 |
| ATTTATCGAG | 376 |
| CCTTTTTCCA | 377 |
| GCACAGAGGT | 378 |
| TGATCTGAAT | 379 |
| GTTGGAGGGA | 380 |
| TTTTGAAGGT | 381 |
| TAAGTCCTAA | 382 |
| GGTGTTAGGG | 383 |
| TGTATGCACC | 384 |
| CCGTGCCATT | 385 |
| GAAATCACCC | 386 |
| TTTGCACGTG | 387 |
| CGTCTGTTTT | 388 |
| CTACACCACA | 389 |
| TGCTACAGGG | 390 |
| GGGAATATAT | 391 |
| TCATGTATTT | 392 |
| TCTCCGTTTA | 393 |
| TACCTCTCGC | 394 |
| GCTTCAACCG | 395 |
| ATGAAGCTAC | 396 |
| CGGTACAACT | 397 |
| GTGTGGTCGT | 398 |
| GGGGTCATGT | 399 |
| AGGCAGCCCA | 400 |
| CAAGCACGAT | 401 |
| TCAAATGGAT | 402 |
| GGACTGAATA | 403 |
| CCGTAGACGT | 404 |
| CGGCGTACCG | 405 |
| GGCGGCGCCC | 406 |
| AGACTTGATC | 407 |
| ACCTTGCACA | 408 |
| TAAGGTGAGT | 409 |
| TTGTTGTTTC | 410 |
| GAGGGAATAC | 411 |
| CTCGTACGCG | 412 |
| CCGCGGTTTA | 413 |
| TTAAAGTTAA | 414 |
| GCATATGGGT | 415 |
| AGTCTGAGCC | 416 |
| TGTCGGTTCG | 417 |
| GGTCTCAACC | 418 |
| GTAACGGCAT | 419 |
| ACACTGAGAA | 420 |
| CCCAACGTCG | 421 |
| AAGAAACTGC | 422 |
| ACCAGCCCAC | 423 |
| TGTAGTTACT | 424 |
| GGCTAGAGGC | 425 |
| GTTCGGCAGA | 426 |
| CCAAAATAGA | 427 |
| CCCATATAAC | 428 |
| GTCACTACCG | 429 |
| GTAGTGTGGC | 430 |
| CAATCTCATA | 431 |
| CCATGTTATA | 432 |
| TAAGCAGTGG | 433 |
| TCGGCGGCTA | 434 |
| TATTAAATGC | 435 |
| GTCGCCATTA | 436 |
| GGCGTCGTTC | 437 |
| CTAGTAGATA | 438 |
| TCGTCAGTAT | 439 |
| GGGGTATCGG | 440 |
| TGCTCTGCCA | 441 |
| TGCCGTAACT | 442 |
| CGGTACAGGC | 443 |
| TCCTAATTTG | 444 |
| TCTTTCTGGA | 445 |
| CCGCGACTTG | 446 |
| ACCTATAGCG | 447 |
| GCCGGCACCT | 448 |
| TTTGATAGGC | 449 |
| ACTGTGAGCT | 450 |
| TTATCGTTCA | 451 |
| ACTAGTGGCC | 452 |
| CCTCCGTGGT | 453 |
| TTAGGGTATG | 454 |
| GAATCAGGCG | 455 |
| GGCTGACCAA | 456 |
| TGCCAGACCG | 457 |
| TCCCTACGCG | 458 |
| TCCGCTGGAG | 459 |
| GGATCAAAAC | 460 |
| TTCACCTCAC | 461 |
| GACACACGGC | 462 |
| TGGGCGATTA | 463 |
| TAAGATCTTC | 464 |
| CTCCGACTAC | 465 |
| GGGCCATCAT | 466 |
| TCAGGCCAGA | 467 |
| CTTGTGGGGC | 468 |
| AGATAGTCTG | 469 |
| GCGTCAAAGT | 470 |
| ACGAAAATTT | 471 |
| GAGTCTGGTG | 472 |
| ATCGAGCGAC | 473 |
| GGTCCTCAGA | 474 |
| TGATTTTGTC | 475 |
| GCATTTCTCA | 476 |
| GCATGCCAGT | 477 |
| ATTAGACGAC | 478 |
| AAAGCCCATA | 479 |
| CACTACATTC | 480 |
| CACGGTTTCT | 481 |
| CCCACCAGTG | 482 |
| CTCACTTGTC | 483 |
| GATAGACTCT | 484 |
| ATTTCCATTT | 485 |
| ATATGTGGCC | 486 |
| CGGGACGAAC | 487 |
| AGAACCGTGA | 488 |
| TAGTGTACTG | 489 |
| AACTAATCGA | 490 |
| CGAAGTGACG | 491 |
| CGGAGCCTCG | 492 |
| ATCACACGAG | 493 |
| CGACGAGTTC | 494 |
| GCTTCCCGTG | 495 |
| GATTCATACC | 496 |
| GAGAGAAGCG | 497 |
| GAAGTGGCCT | 498 |
| GGACGACGCC | 499 |
| TAGGGTCTCA | 500 |
| AACTACAGGT | 501 |
| GTGGCCTGTG | 502 |
| CTTTACCAGC | 503 |
| CGCGTTACTG | 504 |
| TTGCTCCCGT | 505 |
| CATCAAACAA | 506 |
| GCTTTATGAT | 507 |
| CTGCATACTG | 508 |
| GGTGGCTCAG | 509 |
| GGACGATCAA | 510 |
| CCGACTGGTG | 511 |
| GGAACAACCG | 512 |
| GAACGAGACC | 513 |
| CACCAAGAAA | 514 |
| ATGCATTACC | 515 |
| GTATCATGCC | 516 |
| AGTAGATGTT | 517 |
| CTCTAGATGT | 518 |
| GCTACTTGTG | 519 |
| TATGAAACGT | 520 |
| CCTCGTTGAT | 521 |
| CTAGAGCCAT | 522 |
| TAGAGTTATA | 523 |
| AACGAGAGGC | 524 |
| GGTCTACCGT | 525 |
| GCCCCCTCAC | 526 |
| CATAGGAATT | 527 |
| TCCGGCTCGT | 528 |
| TGAGAGTCGG | 529 |
| CGTAGAAATA | 530 |
| CTTTACATGA | 531 |
| GAGCGCCGTC | 532 |
| GGCTCTCGGC | 533 |
| AGAGCTTGTT | 534 |
| AATCAGCCAC | 535 |
| AGAAGAGCCA | 536 |
| TCGTATGAGT | 537 |
| TTCTTCCTCG | 538 |
| ACACAAAAGC | 539 |
| CGCGGGACCC | 540 |
| GTCGCGACAC | 541 |
| CCGGAGGAAA | 542 |
| CGGCGTATGA | 543 |
| TAGGCATTCT | 544 |
| AAAGGAGGGA | 545 |
| ACCTTTACGG | 546 |
| CTACCGTTAA | 547 |
| GAGCTTCGCC | 548 |
| GCCATAGAAG | 549 |
| TTTAGCGTAT | 550 |
| GCAAACAGAT | 551 |
| TAGGTCATGG | 552 |
| CTCTAACAGA | 553 |
| GGCTCATGAA | 554 |
| CAATGTCTCA | 555 |
| TGATCGTATT | 556 |
| GCGCTTTTCA | 557 |
| AAGATTATAT | 558 |
| ACTAGCTGAC | 559 |
| GGTGAGCTCA | 560 |
| CGCTTTCGCT | 561 |
| TGATTCAAAA | 562 |
| ACTGAACAGG | 563 |
| ATTCGAGCTA | 564 |
| TGTAGGCTAA | 565 |
| ACAAAGCTTT | 566 |
| GCCCGAGGGA | 567 |
| GCCCGCTGGG | 568 |
| ACCCCGCTGA | 569 |
| CTTATGCCCT | 570 |
| CCGCCATAGC | 571 |
| CTTAATGATT | 572 |
| CAGTCCACAA | 573 |
| ATGGACGGAC | 574 |
| CGGCCTCTCG | 575 |
| TAGTCGCCAT | 576 |
| GTTGATCTTC | 577 |
| ACTTGCCAAG | 578 |
| ATGACTGGTT | 579 |
| TGTCGTAGGA | 580 |
| AGCAAACACG | 581 |
| TACTGATGAA | 582 |
| GTATCCCATA | 583 |
| TAGCCAGGTT | 584 |
| CGTGTGGCGA | 585 |
| ATCGAATTGC | 586 |
| CCCCAATATT | 587 |
| CCCGTTTCTC | 588 |
| TCCGCATCTA | 589 |
| CAAGCCTCAT | 590 |
| TTTCAATCCC | 591 |
| CCTTCCCATC | 592 |
| AGGTACAAGA | 593 |
| GTGTAATGGA | 594 |
| AAACTGAGCT | 595 |
| ATCTCTGCCC | 596 |
| CGACATTTGC | 597 |
| TGTGAACCCG | 598 |
| TGACACCCCA | 599 |
| TAGGCCAAAG | 600 |
| GAAATTGTAG | 601 |
| GCGTCTGATT | 602 |
| TCTCATTGTT | 603 |
| CTGACATCTC | 604 |
| GTATCCAGTG | 605 |
| GATGGCCGTT | 606 |
| TCACCCTCTC | 607 |
| GGCACTATTC | 608 |
| AAATAACTGT | 609 |
| CAGCTCCATT | 610 |
| CTCTTGACTC | 611 |
| TTTCCTATAC | 612 |
| CCATACCCGA | 613 |
| TCGCCGAGCG | 614 |
| CGCTGAAGCC | 615 |
| TCTGGCCCCA | 616 |
| GCTACATTGA | 617 |
| CGCATCATAA | 618 |
| GCAAAGGGCC | 619 |
| AACGGCGCAG | 620 |
| CGACTGACAT | 621 |
| ATGACAGGGC | 622 |
| CAAGTTCTCC | 623 |
| TCGCCGCTTT | 624 |
| ATGCCGGAAA | 625 |
| GCGGTTACTA | 626 |
| GACATTACAA | 627 |
| CAGAGAGGGC | 628 |
| GCACCGCCTC | 629 |
| CGGTCCGAGC | 630 |
| TGTCCGGTGC | 631 |
| GGTCGGTTGC | 632 |
| GCTCAGCTAA | 633 |
| AGCAGTTCGT | 634 |
| AAATCGATGA | 635 |
| GCTCGGTATG | 636 |
| CCCGCCGCGG | 637 |
| GTGTGATAGG | 638 |
| TTGGACTCCA | 639 |
| TGCTTATCTA | 640 |
| CAAAAGGCGT | 641 |
| TAGGGGGCCT | 642 |
| AAGTATTAAT | 643 |
| GTTTAGCCCG | 644 |
| CGCTAATATG | 645 |
| ACAACACGTT | 646 |
| AGAGATGCTC | 647 |
| TGCCTGATAT | 648 |
| CTTGTAAGTA | 649 |
| CATATTGCCG | 650 |
| CTTAGAAAGT | 651 |
| ATGTTGTATT | 652 |
| CGCATTGAAG | 653 |
| TTATGTTGGT | 654 |
| TCGCCTCAGA | 655 |
| TTCGTTGAGG | 656 |
| GGTGCCGGGC | 657 |
| ACCATTGTAA | 658 |
| TTGATTGTCA | 659 |
| CGGCTCACCT | 660 |
| CTATCACATG | 661 |
| GTAGACAGAA | 662 |
| CCTTTACCAA | 663 |
| GCACATCGAC | 664 |
| TCTCACTTTC | 665 |
| TTCGAGTACT | 666 |
| TAGAAGAGCA | 667 |
| AACCCCACCA | 668 |
| CTGTATCAGT | 669 |
| ACATAATGAG | 670 |
| AGCCTTCCGC | 671 |
| CAGTGCTTTT | 672 |
| TAGTCCGTGT | 673 |
| CGGAATCGGT | 674 |
| CTTGCGGAGA | 675 |
| AAAAATTTGG | 676 |
| TGTTTTCCGC | 677 |
| ATGCTAGGCG | 678 |
| GACTAATTTC | 679 |
| CTGTAGTAAC | 680 |
| CGGATGACTT | 681 |
| TCAGAGTGGA | 682 |
| CAAAATAGCG | 683 |
| GAAGAAGAAG | 684 |
| CACCCGCACG | 685 |
| ACGATGCCCG | 686 |
| CCTACTACAC | 687 |
| ATTGAAACAA | 688 |
| GACCGAAGAT | 689 |
| ACGGCCTGAA | 690 |
| AGGGGAGGTC | 691 |
| CAATCAACTT | 692 |
| GGACAACCGA | 693 |
| TCCCTAAGGC | 694 |
| GTTCTACACG | 695 |
| ACTAACCAGT | 696 |
| GAAGCTGGAT | 697 |
| GGAACCATGG | 698 |
| CTCTACCTGG | 699 |
| TAATGCCTGC | 700 |
| TAAAGGCAAT | 701 |
| CGCCTGGGAA | 702 |
| TCTTGGGGAA | 703 |
| AGAGAGAGAG | 704 |
| GCGTTGGCGC | 705 |
| TTACGACAGA | 706 |
| GGAACTCTTA | 707 |
| GATTGTGGAG | 708 |
| GGGCACTGAT | 709 |
| AGACGCACCA | 710 |
| CCAATTATAA | 711 |
| TAGAGACGCA | 712 |
| CCTCTTGTCG | 713 |
| GAGGAAGCTC | 714 |
| AGTCCCGAGT | 715 |
| TGCTTGCAGT | 716 |
| CCCACTTCCC | 717 |
| CGTTGCCGCG | 718 |
| CCCCTGGTTC | 719 |
| ACGACCAATA | 720 |
| CTTAGGGTTC | 721 |
| AAACATATCA | 722 |
| GGGTCGTAGA | 723 |
| CTCCGTAGCG | 724 |
| CTGGTCATAA | 725 |
| TTGACAGATC | 726 |
| GAGTAAAGTC | 727 |
| ATATGGGCTT | 728 |
| TACAACTACT | 729 |
| AATTCAGCCG | 730 |
| GATTGTACTA | 731 |
| TCGTAATGCG | 732 |
| CGATAACTGC | 733 |
| AACTTGGCGG | 734 |
| CGTGGATGTA | 735 |
| CCTTCCCGAA | 736 |
| CTAAACCCGT | 737 |
| CAACATTCCC | 738 |
| CTTACCCTCT | 739 |
| GGAAAGTTCT | 740 |
| CGGATTGGCT | 741 |
| AATGTAGGGC | 742 |
| AATGAATCGC | 743 |
| ATCATACACC | 744 |
| AGTTGGGCAG | 745 |
| AGAAGAAGGG | 746 |
| GCGTGCGCTA | 747 |
| CCCCGATAAA | 748 |
| TACCAAGTGC | 749 |
| TGTGTTTTCG | 750 |
| CCCAGATGTC | 751 |
| GCGAGCTTCC | 752 |
| GTGTCACGTA | 753 |
| ATAGGCCGAG | 754 |
| GAGCTACCAG | 755 |
| CGCGGCGGAG | 756 |
| TCTTGCACGA | 757 |
| TGCCCTAAAG | 758 |
| TTGCGCTTTG | 759 |
| CATATAAAGG | 760 |
| AATAGCGAAT | 761 |
| TACGCTAAGG | 762 |
| ACTTAGTTCG | 763 |
| CGTGCGGAAC | 764 |
| ACCCGATTCG | 765 |
| TGCAGAGTTT | 766 |
| GAATCATTAG | 767 |
| AGTACACTGG | 768 |
| TTGTGCGGTT | 769 |
| ATGACATGCA | 770 |
| TTCTCGGACG | 771 |
| AGATTGAAGA | 772 |
| GGCGGACTGT | 773 |
| TTTATGGTAA | 774 |
| CAGTAGGGTG | 775 |
| GACAGGCAAG | 776 |
| GATGTGTCGT | 777 |
| ACTTGACGGA | 778 |
| AAGTCCGAAA | 779 |
| TGGGTGTAGG | 780 |
| ACTTACCGCG | 781 |
| CTGTGCACCC | 782 |
| ATTGCTCTCT | 783 |
| CAGAAGACAA | 784 |
| TTACGCTATA | 785 |
| ACGTGGAAAT | 786 |
| TGAGGCTGGT | 787 |
| ATTATGAGAT | 788 |
| GACTTGTAGT | 789 |
| TCGCTGAGGA | 790 |
| CCCAACTCTA | 791 |
| GATAGGGAGG | 792 |
| TAGAAATCAG | 793 |
| GTCGCTAGAA | 794 |
| AAAATAGAAA | 795 |
| GCTCCTGGGT | 796 |
| CGCGCTCGCG | 797 |
| GGCAAACGCA | 798 |
| TTTACTACCT | 799 |
| ATCCTAAACT | 800 |
| CTCCGTATGT | 801 |
| TATCGTCCAG | 802 |
| GCCGGCGGTA | 803 |
| TGCTCCATTT | 804 |
| TGGCTGTTGT | 805 |
| TACTGCGCAA | 806 |
| TATACGGCTT | 807 |
| GGTTATTACC | 808 |
| ATCAGGAGGA | 809 |
| CTATTGCCAG | 810 |
| ACGTACACAC | 811 |
| CAGCCTAGCT | 812 |
| GAAAAACAAC | 813 |
| CGTTCAGTTA | 814 |
| CAATCAGAAT | 815 |
| GGGCTACTCT | 816 |
| CCCCATTGGG | 817 |
| TAGGGAACGG | 818 |
| CAGCTGATAC | 819 |
| ATTCCTGTGA | 820 |
| TCAGAGCCGT | 821 |
| CATGAAAAGC | 822 |
| TGACCTGTGA | 823 |
| GCATTAGCAG | 824 |
| GACAGAACCA | 825 |
| TCCAGTATAT | 826 |
| TGTTCCGCTA | 827 |
| GATATCCATT | 828 |
| CATATGGACC | 829 |
| GATATAGTAA | 830 |
| CACCTTTTTT | 831 |
| AGCTTGCGGG | 832 |
| CGCACAGGGA | 833 |
| TCTGGGTGCT | 834 |
| TGAGTCGTTT | 835 |
| TTACAATGTG | 836 |
| CTTGCAAACA | 837 |
| TGTCGAGCTG | 838 |
| ACTTTAACCT | 839 |
| ATATAAGTGC | 840 |
| GGAAGGGCGT | 841 |
| TTTGACTTGA | 842 |
| GTATAAACGG | 843 |
| TAACCGGATG | 844 |
| TTCTCATCAG | 845 |
| CTCGGTTACG | 846 |
| ATATGGTTCT | 847 |
| CGCCCCCGAA | 848 |
| ACCTCGATCG | 849 |
| CTCGAATAAT | 850 |
| GCCCGAGCTT | 851 |
| AACAGTCAAC | 852 |
| CTGGAACCTC | 853 |
| AATAACGGGG | 854 |
| ACGCCCCACT | 855 |
| GGCAACATGA | 856 |
| GCTATTTCGC | 857 |
| TTCCACTTTA | 858 |
| GCCGATGGAT | 859 |
| AAGTTGGTAA | 860 |
| CACTAGCTAG | 861 |
| ACATGCCCCT | 862 |
| TTCATTACTC | 863 |
| GGTTTAATAT | 864 |
| CCTGCAGTGA | 865 |
| TCTTTAAGTT | 866 |
| TGGCGATCGA | 867 |
| CTTTTTAGCT | 868 |
| CCCAGTCTCT | 869 |
| AAATGTTTCG | 870 |
| ATATAAGACG | 871 |
| TCACTTTACA | 872 |
| CCTGGCGCCC | 873 |
| GGATTACTGG | 874 |
| GAATGATCTT | 875 |
| GCTCGGATCG | 876 |
| CAGCTGCGAG | 877 |
| ACCCTTACTA | 878 |
| AGGTGAAACT | 879 |
| CGAATTTGAT | 880 |
| CGCTGTGCGG | 881 |
| TTACCGCACC | 882 |
| GGAATCTTAA | 883 |
| CTCAACACCC | 884 |
| CGTGCCCTTG | 885 |
| GCAGGCTCGA | 886 |
| ACCAACGAAG | 887 |
| CCTGTAATTT | 888 |
| GGGTGGGATG | 889 |
| TTGCTCACCG | 890 |
| TTACGACCAC | 891 |
| TTTTCTAACC | 892 |
| GCTTTAGATA | 893 |
| CACGTATTGG | 894 |
| AAATATCTCC | 895 |
| GCTGGAAAAC | 896 |
| GAGCGCATTA | 897 |
| GTGGAGGGGT | 898 |
| TCCACTGGGA | 899 |
| CAATAGCGGA | 900 |
| CATCTAGTTT | 901 |
| GAAGTTCCGG | 902 |
| AGCGAGATTC | 903 |
| TTAAGGTCGG | 904 |
| AATGGTTAGG | 905 |
| CGTTATTATA | 906 |
| ACGGAAAGGA | 907 |
| CCTTGTCCCG | 908 |
| ATACTTTTTT | 909 |
| CTGGGTCTGG | 910 |
| AACCATTGCG | 911 |
| AGACCGGGCC | 912 |
| TGGGACACAC | 913 |
| TGCGCAGTTG | 914 |
| CGTTCGCCTT | 915 |
| TCTCACTCGT | 916 |
| ACACCGACGT | 917 |
| TTCAGCCCCT | 918 |
| AGGCGACTAA | 919 |
| TGCTATCAAG | 920 |
| GTCCAGTAGC | 921 |
| CGTGTGGGCG | 922 |
| GTGGTTCTCC | 923 |
| GCAGCCGACG | 924 |
| GCTGTCCACG | 925 |
| CGACACTCAT | 926 |
| CATGGCACCT | 927 |
| TGTGACGTGT | 928 |
| TTTGGACTAA | 929 |
| TTCATGCCCG | 930 |
| TTGATCGTGG | 931 |
| TAGCATAGGA | 932 |
| GTAGTTGCAA | 933 |
| GGGACAGCTA | 934 |
| AAACCCCCAA | 935 |
| ACTCTCACAA | 936 |
| ATCATTGCCA | 937 |
| CCAGTTTGCG | 938 |
| ACATTAGTCA | 939 |
| CTCCAGGGTA | 940 |
| GAAGGGCCAA | 941 |
| CAGTCTCCCC | 942 |
| GAGACATTCC | 943 |
| AACGGTGTTG | 944 |
| AGCATTATCA | 945 |
| CTATACCGAG | 946 |
| AACTGGATCA | 947 |
| GTCTTGTCGG | 948 |
| GACGAGCCGC | 949 |
| GGAACACTGT | 950 |
| TAAATGCGTT | 951 |
| GCGAACACAG | 952 |
| TTCTCTCAAC | 953 |
| GTCGTACTGA | 954 |
| TGTGGCGTAA | 955 |
| TGAGCGGCGT | 956 |
| CCTCGTGAAC | 957 |
| GAGCAATGAA | 958 |
| CGAGACCTAA | 959 |
| AACTGAGCGC | 960 |
| TAAAGCTCGT | 961 |
| CTCTTTACGT | 962 |
| CCCCGTGGAA | 963 |
| TCGGTTCGTC | 964 |
| CTGCTTACAC | 965 |
| ACACCGTAAT | 966 |
| CCTGGTCGGC | 967 |
| GGTTATTTGG | 968 |
| GCAACTGAGT | 969 |
| ATAAGGCCTC | 970 |
| CGTGCGAAGG | 971 |
| GTCACACACT | 972 |
| CATACGGCAA | 973 |
| GAACTGCCCA | 974 |
| AATATGTGAA | 975 |
| CCGATCCTGT | 976 |
| CAAAGAGCCT | 977 |
| TAACTTAGAG | 978 |
| CAGCATGTAG | 979 |
| CCCCATGCAG | 980 |
| TCTGAACCAC | 981 |
| GCGTGCAAAA | 982 |
| GCTAGTACCG | 983 |
| TTTCCCGCGC | 984 |
| CCTTAGTAGG | 985 |
| TTGTGTCTTG | 986 |
| GCAACGAAGC | 987 |
| TGAAACCCTT | 988 |
| TTCTACGATC | 989 |
| ATTAAAGGTG | 990 |
| TATCTAACGG | 991 |
| AGTGCTCCTG | 992 |
| CCGTCCCTCT | 993 |
| CTAACGAGCG | 994 |
| AAGTCCGGCT | 995 |
| GGCGTATAAG | 996 |
| AGATATTAGG | 997 |
| TCCTAACAGC | 998 |
| GAGGATACGC | 999 |
| CGCTCTTTAA | 1000 |
| ACCGGCAGGC | 328 |
| GCTAAAATCT | 329 |
| GCCGTTGACG | 330 |
| GGAGTTGTTG | 331 |
| TACTTGAGAA | 332 |
| CGGGTGCGCT | 333 |
| AAAAGCGTCT | 334 |
| GTAAAGATAG | 335 |
| GCCTGGTCAG | 336 |
| GGCAAAAAGG | 337 |
| ACCCTTCTCT | 338 |
| TCACATAGTG | 339 |
| TCGTCTGTGC | 340 |
| TGCTCGGATC | 341 |
| GGCGTATAAG | 996 |
| AGATATTAGG | 997 |
| TCCTAACAGC | 998 |
| GAGGATACGC | 999 |
| CGCTCTTTAA | 1000 |

In another embodiment, a random sequence fragment can be linked to the 5′ and/or the 3′ end of the barcode and the random sequence fragment can, for example, be used for bioinformatic removal of PCR duplicates. The random sequence fragment can also be used to add length to the nucleic acid construct and can serve as a marker for bioinformatic analysis to identify the beginning or the end of the barcode after sequencing. In another embodiment, the nucleic acid barcode construct comprises at least a first and a second random sequence fragment, and the first random sequence fragment can be linked to the 5′ end of the barcode and the second random sequence fragment can be linked to the 3′ end of the barcode. In another embodiment, one or at least one random sequence fragment is linked to the 5′ and/or the 3′ end of the barcode. In one aspect, the random sequence fragments can be extended as needed to make the nucleic acid barcode construct longer for different applications such as whole genome sequencing where short inserts may be lost.

In various embodiments, the random sequence fragments can be from about 5 to about 20 bases in length, about 5 to about 19 bases in length, about 5 to about 18 bases in length, about 5 to about 17 bases in length, about 5 to about 16 bases in length, about 5 to about 15 bases in length, about 5 to about 14 bases in length, about 5 to about 13 bases in length, about 5 to about 12 bases in length, about 5 to about 11 bases in length, about 5 to about 10 bases in length, about 5 to about 9 bases in length, about 5 to about 8 bases in length, about 6 to about 10 bases in length, about 7 to about 10 bases in length, or about 8 to about 10 bases in length.

In another illustrative aspect, the barcode may be flanked by primer binding sequences (i.e., directly or indirectly linked to the barcode) so that the nucleic acid barcode construct comprising the barcode, and any attached random sequence, can be amplified during a polymerase chain reaction (PCR) and/or sequencing protocol. In one aspect, the primer binding sequences can be useful for binding to one or more universal primers or a universal primer set. In one illustrative embodiment, the universal primers can contain overhang sequences that enable attachment of index adapters for sequencing. In one embodiment, the adapters can be NGS adapters (e.g. Illumina) positioned internally but towards the end of either the 5′ or the 3′ primer, not as the terminating structure, to avoid the formation of primer dimers. In this aspect, the primers can be any primers of interest. In this embodiment, the first primer binding sequence can be linked at its 3′ end to the 5′ end of a first random sequence fragment and the second primer binding sequence can be linked at its 5′ end to the 3′ end of a second random sequence fragment with the barcode between the random sequence fragments. In another embodiment, the first primer binding sequence can be linked at its 3′ end to the 5′ end of the barcode and the second primer binding sequence can be linked at its 5′ end to the 3′ end of a random sequence fragment linked to the 3′ end of the barcode. In another embodiment, the first primer binding sequence can be linked at its 3′ end to the 5′ end of a random sequence fragment and the second primer binding sequence can be linked at its 5′ end to the 3′ end of the barcode where the barcode is linked at its 5′ end to the 3′ end of the random sequence fragment. In yet another embodiment, the first primer binding sequence can be linked at its 3′ end to the 5′ end of the barcode and the second primer binding sequence can be linked at its 5′ end to the 3′ end of the barcode.

Primer binding sequences used in accordance with the present invention can range in length from about 15 bases to about 30, from about 15 bases to about 29 bases, from about 15 bases to about 28 bases, from about 15 bases to about 26 bases, from about 15 bases to about 24 bases, from about 15 bases to about 22 bases, from about 15 bases to about 20 bases, 16 bases to about 28 bases, from about 16 bases to about 26 bases, from about 16 bases to about 24 bases, from about 16 bases to about 22 bases, from about 16 bases to about 20 bases, 17 bases to about 28 bases, from about 17 bases to about 26 bases, from about 17 bases to about 24 bases, from about 17 bases to about 22 bases, from about 17 bases to about 20 bases, 18 bases to about 28 bases, from about 18 bases to about 26 bases, from about 18 bases to about 24 bases, from about 18 bases to about 22 bases, or from about 18 bases to about 20 bases.

An exemplary sequence of a nucleic acid barcode construct is shown below. The /5AmMC6/ is a 5′ amine modification for attachment to the DNAFile. The *'s are phosphorothioate bond modifications for stability. The A*G*A*CGTGTGCTCTTCCGATCT sequence (SEQ ID NO: 1001) is the 5′ primer binding sequence. The GCTACATAAT (SEQ ID NO: 1) is an exemplary barcode sequence. The N's represent the random sequence fragment. The AGATCGGAAGAGCGTCG*T*G*T (SEQ ID NO: 1002) is the 3′ primer binding sequence.

(SEQ ID NO: 1003)
/5AmMC6/A*G*A*CGTGTGCTCTTCCGATCTGCTACATAATNNN
NNNNNNNAGATCGGAAGAGCGTCG*T*G*T.

In all of the various embodiments described above, the entire nucleic acid barcode construct can range in length from about 30 bases to about 350 bases, from about 30 bases to about 300 bases, from about 30 bases to about 270 bases, about 30 bases to about 240 bases, about 30 bases to about 230 bases, about 30 bases to about 220 bases, about 30 bases to about 210 bases, about 30 bases to about 200 bases, about 30 bases to about 190 bases, about 30 bases to about 180 bases, about 30 bases to about 170 bases, about 30 bases to about 160 bases, about 30 bases to about 150 bases, about 30 bases to about 140 bases, about 30 bases to about 130 bases, about 30 bases to about 120 bases, from about 30 bases to about 110 bases, from about 30 bases to about 100 bases, from about 30 bases to about 90 bases, from about 30 bases to about 80 bases, from about 30 bases to about 70 bases, from about 30 bases to about 60 bases, from about 30 bases to about 50 bases, from about 30 bases to about 40 bases, 40 bases to about 120 bases, from about 40 bases to about 110 bases, from about 40 bases to about 100 bases, from about 40 bases to about 90 bases, from about 40 bases to about 80 bases, from about 40 bases to about 70 bases, from about 40 bases to about 60 bases, from about 40 bases to about 50 bases, 50 bases to about 120 bases, from about 50 bases to about 110 bases, from about 50 bases to about 100 bases, from about 50 bases to about 90 bases, from about 50 bases to about 80 bases, from about 50 bases to about 70 bases, from about 50 bases to about 60 bases, or about 42 bases to about 210 bases.

### EXEMPLARY EMBODIMENTS

In accordance with embodiment 1, a library comprising a plurality of origami folded DNA data storage files is provided wherein each of said DNAFiles comprises
a single stranded DNA scaffold; and
a plurality of single stranded DNA staple oligonucleotides that bind through complementary base pairing with a segment of the DNA scaffold, wherein said staple oligonucleotides cause the DNA scaffold to reversibly fold into a two or three dimensional shape, and said DNA scaffold and/or one or more of said staple oligonucleotides comprise nucleic acid sequences that encode digital information, wherein the individual DNAFiles differ from one another based on the nucleic acid sequence of the staple oligonucleotides bound to the DNA scaffold of each DNAFile.

In accordance with embodiment 2 the library of embodiment 1 is provided wherein one or more of said staple oligonucleotides comprise nucleic acid sequences that encodes digital information, and the DNA scaffold does not encode digital information.

In accordance with embodiment 3 the library of embodiment 1 or 2 is provided wherein said one or more of said staple oligonucleotides have a length of about 30 to 200 nucleotides and comprise a nucleic acid sequence non-complementary to said DNA scaffold, wherein the non-complementary nucleic acid sequence comprises a nucleic acid sequence that encodes digital information.

In accordance with embodiment 4 the library of embodiment 3 is provided wherein said nucleic acid sequences that encode digital information comprise two primer binding sequences that flank the non-complementary nucleic acid sequences that encode digital information, wherein a first primer binding sequence is located at the 5′ terminus of the non-complementary nucleic acid sequence and a second primer binding sequence is located at the 3′ terminus of the non-complementary nucleic acid sequence.

In accordance with embodiment 5 the library of any one of embodiments 1˜4 is provided wherein the 3′ end of the staple oligonucleotides are modified to stabilized and prevent undesirable interactions, optionally wherein the modification comprises the addition of a poly A or poly T extension or modification of the 3′ terminal nucleic acids of the staple oligonucleotides.

In accordance with embodiment 6 a library comprising a plurality of origami folded DNA data storage files (DNAFiles) is provided wherein, each of said DNAFiles comprises
a single stranded DNA scaffold; and
a plurality of single stranded DNA staple oligonucleotides, each of said staple oligonucleotides comprising nucleic acid sequences that bind through complementary base pairing with two non-contiguous segments of the DNA scaffold, wherein said staple oligonucleotides cause the DNA scaffold to fold into a two or three dimensional shape having a first surface;
a plurality of data oligonucleotides, said data oligonucleotides comprising a sequence complementary to a nucleic acid sequence of the single stranded DNA scaffold, a nucleic acid sequence that encodes digital information, and a first and second primer binding sequence, wherein the first primer binding sequence is locate 5′ to the digital information encoding nucleic acid sequence, the second primer binding sequence is locate 3′ to the digital information encoding nucleic acid sequence, and said plurality of data oligonucleotides are localized to said first surface, and the individual DNAFiles differ from one another based on the nucleic acid sequence of the data oligonucleotides bound to the DNA scaffold of each DNAFile.

In accordance with embodiment 7 the library of embodiment 6 is provided wherein said staple oligonucleotides cause the DNA scaffold to reversibly fold into a multi-layered sheet conformation having a top surface and a bottom surface, wherein said plurality of data oligonucleotides are only linked to, and project away from, the top surface, optionally wherein the data oligonucleotides are uniformly distributed over said top surface.

In accordance with embodiment 8 the library of embodiment 6 or 7 is provided wherein each DNAFiles comprises a scaffold DNA folded into a bilayer sheet conformation comprising two symmetrical layers of origami DNA, optionally wherein the data oligonucleotides are linked to the folded DNA scaffold in a manner that the non-complementary single strands of the data oligonucleotides are uniformly distributed over said top surface at a density selected from the range of 20% to 100% of total occupancy, optionally wherein the data oligonucleotides are are uniformly distributed over said first surface at a density of 20%, 40%, 60%, 80%, or 100 of total occupancy, optionally at a density of less than 500, 300, 200, 100, 50, 40, 20 or 10 data oligonucleotides per 100 nm², optionally wherein the non-complementary single strands of the data oligonucleotides, at the point where they project from the exterior surface of the folded DNA scaffold, are separated from one another by an average minimum distance of about 3 nm to about 18 nm, about 6 nm to about 18 nm, about 6 nm to about 12 nm, about 9 nm to about 12 nm, or about 7 nm to about 11 nm.

In accordance with embodiment 9 the library of any one of embodiments 6-8 is provided wherein the shape of each DNAFile is stabilized by
a) adding a sequence of six or more thymidine resides (poly(T)) to the end of the noncomplementary sequence of the data oligonucleotides;
b) decreasing the length of the staple oligonucleotides that are located near sheet corners to less than 100 nucleotides, or less than 50 nucleotides, to allow for flexibility during fold process;
c) adding additional crossover staple oligonucleotides that bind to noncontiguous sequences of the DNA scaffold to improve stability and shape of the origami folded construct;
d) introducing intentional gaps or missing base pairs within the scaffold DNA strand/staple folded structure (i.e. “skips”) near the center-line of the folded multi-layered sheet to decrease twist;
e) any combination of a) through d).

In accordance with embodiment 10 the library of any one of embodiments 1-9 is provided wherein each DNAFiles comprises about 200-300 staple oligonucleotides.

In accordance with embodiment 11 the library of any one of embodiments 1-10 is provided wherein the data oligonucleotides have a length of about 100 to 200 nucleotides and comprise a nucleic acid sequence complementary to said DNA scaffold and a nucleic acid sequence non-complementary to said DNA scaffold, wherein the non-complementary nucleic acid sequence encodes digital information, further wherein said non-complementary nucleic acid sequence does not participate in the folding of the DNA scaffold into a two or three dimensional shape, optionally wherein said non-complementary nucleic acid sequence is flanked by a first primer binding sequence and second primer binding sequence, optionally wherein the non-complementary nucleic acid sequence has a length of at least 50 nucleotides, optionally a length from about 60 nucleotides to about 180 nucleotides.

In accordance with embodiment 12 the library of any one of embodiments 1-10 is provided wherein the staple oligonucleotides have a length of about 100 to 200 nucleotides and comprise a first nucleic acid sequence complementary to said DNA scaffold and a second nucleic acid sequence complementary to said DNA scaffold, wherein the first and second sequences are complementary to non-contiguous sequences of the DNA scaffold, optionally wherein the first and second sequences are linked to one another by a linker nucleic acid sequence that is not complementary with the sequence of the DNA scaffold.

In accordance with embodiment 13 the library of any one of embodiments 1-12 is provided where the nucleic acid sequences having complementarity to said DNA scaffold, present in the staple oligonucleotides and the data oligonucleotides, represent nucleic acid sequences having at least 85%, 90%, 95% or 99% sequence identity to a nucleic acid sequence of the DNA scaffold, optionally wherein the nucleic acid sequences having complementary to said DNA scaffold have 100% sequence identity to a nucleic acid sequence of the DNA scaffold.

In accordance with embodiment 14 the library of any one of embodiments 1-13 is provided wherein each member of said plurality of origami folded DNAFiles comprises a different single stranded DNA scaffold.

In accordance with embodiment 15 the library of any one of embodiments 1-13 is provided wherein each member of said plurality of origami folded DNAFiles have the same single stranded DNA scaffold but differ from each other based on the sequence of the data oligonucleotides associated with each DNAFile.

In accordance with embodiment 16 the library of any one of embodiments 1-14 is provided wherein each member of said plurality of origami folded DNAFiles has a unique shape.

In accordance with embodiment 17 the library of any one of embodiments 1-16 is provided wherein each origami folded DNAFile further comprises a linked unique nucleic acid barcode construct.

In accordance with embodiment 18 the library of any one of embodiments 1-16 is provided wherein subsets of the origami folded DNAFiles of the library are linked to a nucleic acid barcode construct unique to each subset, but different between the subsets.

In accordance with embodiment 19 the library of any one of embodiments 17-18 is provided wherein the nucleic acid barcode construct is associated with the origami DNAFile via base-pairing.

In accordance with embodiment 20 the library of embodiment 19 is provided wherein the base-pairing occurs between
i) a sequence of a single-stranded non-complementary region of one or more of said staple oligonucleotides and a complementary sequence linked to the nucleic acid barcode construct; or
ii) a sequence of the single stranded DNA scaffold, optionally a single-stranded non-complementary region extending from the 5′ or 3′ end of the DNA scaffold, and a complementary sequence linked to the nucleic acid barcode construct.

In accordance with embodiment 21 the library of any one of embodiments 1-20 is provided wherein the nucleic acid barcode construct is associated with the DNAFile by a high affinity, non-covalent bond interaction between a biotin molecule linked to the 5′ and/or the 3′ end of the nucleic acid barcode construct and a molecule that binds to biotin, said molecule being linked to the DNAFile.

In accordance with embodiment 22 the library of any one of embodiments 1-21 is provided wherein the data oligonucleotides of each individual origami folded DNAFile of said library comprises an identical set of PCR binding sequences for preselected PCR primers, where the PCR binding sequences differ between the data oligonucleotides of each respective DNAFile file of the library.

In accordance with embodiment 23 the library of any one of embodiments 1-21 is provided wherein the data oligonucleotides of each origami folded DNAFile of said library comprises a unique set of PCR binding sequences for preselected PCR primers.

In accordance with embodiment 24 the library of any one of embodiments 1-23 is provided wherein said data oligonucleotides comprise a first and second primer binding sequence located at the respective 5′ and 3′ ends of the nucleic acid sequence encoding said digital information and said sequence complementary to a nucleic acid sequence of the single stranded DNA scaffold is linked 5′ to the first primer binding sequence or 3′ to the second primer binding sequence.

In accordance with embodiment 25 the library of any one of embodiments 1-23 is provided wherein said data oligonucleotides comprise a first and second primer binding sequence located at the respective 5′ and 3′ ends of each data oligonucleotide, wherein both the 5′ end of the data oligonucleotide and the 3′ end of the data oligonucleotide are non-complementary to the DNA scaffold, optionally wherein the percent occupancy of the data oligonucleotides on the DNA scaffold is less than 100% and optionally less than 50%.

In accordance with embodiment 26 the library of any one of embodiments 7-25 is provided wherein the data oligonucleotides are bound only to the top surface, and the non-complementary sequences of the data oligonucleotides (overhang) project away from the DNA scaffold in approximately the same direction optionally at an angle within 70 to 90 degrees or within 80 to 90 degrees of the planar surface of the top surface.

In accordance with embodiment 27, the library of any one of embodiments 7-26 is provided wherein the density of the data oligonucleotides on the top surface is about 30, 40, 50, 60, 70, 80, or 90 percent maximal occupancy, or at a density of less than 500, 300, 200, 100, 80, 50, 40, 20 or 10 data oligonucleotides per 100 nm², optionally at a density of 50, 40, 20 or 10 data oligonucleotides per 100 nm².

In accordance with embodiment 28 a method of retrieving digital data stored in DNA is provided wherein the method comprises
providing a library of origami folded DNAFiles of any one of embodiments 1-27;
denaturing a folded origami DNAFile of said library to at least partially disrupt the hybridized duplex between the single stranded staple oligonucleotides, the data oligonucleotides and the DNA scaffold;
conducting PCR amplification on nucleic acid sequences of said denatured DNA scaffold, data oligonucleotides and staple oligonucleotides to produce amplicons;
reannealing the staple oligonucleotides and the data oligonucleotides with the DNA scaffold to reconstitute the folded origami DNAFile;
separating the amplicons from the reconstituted folded origami DNAFile;
returning the reconstituted folded origami DNAFile to the library; and
sequencing the amplicons to retrieve digital data encoded by the DNAFile.

In accordance with embodiment 29 the method of embodiment 28 is provided wherein said denaturing step completely releases all staple oligonucleotides and all data oligonucleotides as free single stranded nucleic acids.

In accordance with embodiment 30 the method of any one of embodiments 28-29 is provided wherein the amplicons are separated from the reconstituted folded origami DNAFiles via gel electrophoresis.

In accordance with embodiment 31 the method of any one of embodiments 28-29 is provided wherein the amplicons are separated from the reconstituted folded origami DNAFiles via size exclusion chromatography.

In accordance with embodiment 32 the method of any one of embodiments 28-31 is provided further comprising the step of confirming the correct size and shape of the reconstituted folded origami DNA scaffold prior to returning the reconstituted folded origami DNA scaffold to the library.

In accordance with embodiment 33 the method of any one of embodiments 28-32 is provided further comprising the step of selecting one or more individual origami folded DNAFiles from the other origami folded DNAFiles of said library and conducting the denaturing step only on the selected origami folded DNAFiles.

In accordance with embodiment 34 the method of any one of embodiments 28-33 is provided wherein the one or more individual origami folded DNAFiles are selected based on selective binding of individual origami folded DNAFiles to a complementary oligonucleotide immobilized on a solid surface, or to a complementary oligonucleotide bound to a magnetic or fluorescently labelled nanoparticle.

In accordance with embodiment 35 a method of storing digital information using DNA as the storage medium is provided wherein the method comprising the steps:
providing a single stranded DNA scaffold;
providing a plurality of single stranded staple oligonucleotides and data oligonucleotides that bind through complementary base pairing with a segment of the DNA scaffold, wherein the staple oligonucleotides cause the DNA scaffold to fold into a two or three dimensional shape, wherein the data oligonucleotides comprise nucleic acid sequences that encode digital information;
mixing said DNA scaffold and said staple oligonucleotides and data oligonucleotides under conditions that allow sequence specific hybridization of the staple oligonucleotides and data oligonucleotides to the DNA scaffold and folding of the DNA scaffold.

In accordance with embodiment 36 the method of embodiment 35 is provided wherein said data oligonucleotide comprises two primer binding sequences that flank the non-complementary nucleic acid sequences that encode digital information, wherein a first primer binding sequence is at the 5′ terminus of the nucleic acid sequence that encodes digital information and a second primer binding sequence is at the 3′ terminus of the nucleic acid sequence that encodes digital information.

### Example 1

Fold 2D/Wireframe Structure with Overhangs Coding for Data

DNA scaffolds were designed and folded into planar parallel 2D bilayer sheets to maximize data incorporation surface area, stability and overhang positions. The parallel design showed twisting upon simulating, and new elements were introduce to decrease twist, particularly by introducing intentional gaps or missing base pairs within the scaffold DNA strand/staple folded structure (i.e. “skips”) near the center-line of the folded multi-layered sheet. The sheets were folded using standard techniques with 10:1 staple:scaffold ratio, 12.5 mM salt MgCl₂ concentration and a 14 hr thermal ramp.

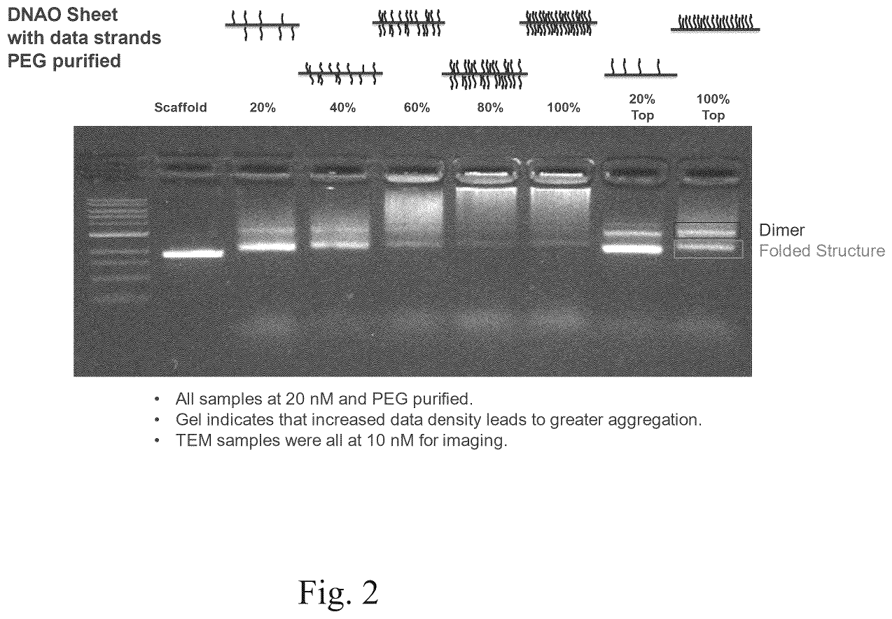

The DNA scaffolds were designed to accommodate 80 data oligonucleotides with the oligonucleotides attached to both the top and bottom surface of the folded 2D bilayer sheets. The data oligonucleotides were prepared having a total length of 80 nucleotides, with a 20 nucleotide sequence having complementarity with a corresponding sequence of the DNA scaffold, two primer binding sequences of 20 nucleotides each that flank a 9 nucleotide sequence encoding data. Folded sheets were prepared having different combinations of data strand occupancy to test most stable configuration. Specifically, embodiments were prepared where the folded sheet had 20%, 40%, 60%, 100% occupancy on both the top and bottom, or alternatively the folded sheet had 20% or 100% occupancy on the top sheet only (see FIG. 2). The 100% double sided occupancy embodiment comprises a total of 720 data bases (80 data oligonucleotides X 9 nucleotides) and the 100% single sided occupancy embodiment comprises a total of 360 data bases (40 data oligonucleotides X 9 nucleotides).

Accordingly, a 100 ul volume of 20 nM solution of DNAFiles containing 100% single sided occupancy provides 4.35×10¹⁴ data bases or 1.45×10¹⁴ bits of data.

The results provided in FIG. 2 demonstrate that the presence of data strands induces a degree of aggregation correlated to the % occupancy. One sided occupancy resulted in substantially less muti-order structures. PCR and sequencing was performed to assess error in occupancy dependent errors in data incorporation or reading. All data strands had 1:1 incorporation in the designed location (i.e., no mis-matched incorporation was detected). Lower occupancy was associated with higher sequence reads from occupied locations (presumably due to less steric hindrance). The 100% single sided occupancy embodiment had a 2.4× higher total sequence read count than the 100% double sided occupancy embodiment DS.

1. A library comprising a plurality of origami folded DNA data storage files (DNAFiles), each of said DNAFiles comprising
a single stranded DNA scaffold; and
a plurality of single stranded DNA staple oligonucleotides that each bind through complementary base pairing with two non-contiguous nucleic acid sequences of the DNA scaffold, wherein said staple oligonucleotides cause the DNA scaffold to fold into a two or three dimensional shape having a first surface;
a plurality of data oligonucleotides, said data oligonucleotides comprising a sequence complementary to a nucleic acid sequence of said single stranded DNA scaffold, a nucleic acid sequence that encodes digital information, and a first and second primer binding sequence, wherein the first primer binding sequence is 5′ to the digital information encoding nucleic acid sequence, the second primer binding sequence is 3′ to the digital information encoding nucleic acid sequence, wherein the individual DNAFiles differ from one another based on the nucleic acid sequence of the plurality of data oligonucleotides bound to the DNA scaffold of each DNAFile.

2. The library of claim 1 wherein
said first primer binding sequence is located at the 5′ terminus of said data oligonucleotides and said second primer binding sequence is located at the 3′ terminus of said data oligonucleotides

3. The library of claim 2 wherein each DNAFile has a bilayer sheet conformation comprising two symmetrical layers of origami DNA,
wherein the shape of each DNAFile is stabilized by
a) adding a sequence of six or more thymidine resides (poly(T)) to the end of the noncomplementary sequence of the data oligonucleotides;
b) decreasing the length of staple oligonucleotides located near sheet corners to less than 100 nucleotides, or less than 50 nucleotides, to allow for flexibility during the folding process;
c) introducing intentional gaps or missing base pairs within the scaffold DNA strand/staple folded structure (i.e. “skips”) near the center-line of the folded multi-layered sheet; or
d) any combination of a) through c).

4. The library of claim 3 wherein said data oligonucleotides have a length of about 30 to 200 nucleotides, and the first and second primer binding sequences, and the sequence complementary to a nucleic acid sequence of said single stranded DNA scaffold, are each independently 10 to 20 nucleotides in length, and the digital information encoding nucleic acid sequence is at least 50 nucleotides in length.

5. The library of claim 1 wherein
said nucleic acid sequence of the data oligonucleotide that is complementary to said single stranded DNA scaffold is 5′ to said first primer binding sequence, or 3′ to said second primer binding sequence.

6. The library of claim 1 wherein each member of said plurality of origami folded DNAFiles comprises a different single stranded DNA scaffold.

7. The library of claim 1 wherein
i) each member of said plurality of origami folded DNAFiles has a unique shape; or
ii) each origami folded DNAFile further comprises a linked unique nucleic acid barcode construct; or
iii) both i) and ii).

8. The library of claim 1 wherein each origami folded DNAFile further comprises a unique nucleic acid barcode construct linked to the origami DNAFile via base-pairing, wherein said base-pairing that links the nucleic acid barcode construct with the origami DNAFile occurs between
i) a single-stranded non-complementary nucleic acid sequence of one or more of said staple oligonucleotides and a complementary sequence linked to the nucleic acid barcode construct; or
ii) a single-stranded non-complementary nucleic acid sequence extending from the 5′ or 3′ end of the single-stranded DNA scaffold and a complementary sequence linked to the nucleic acid barcode construct.

9. The library of claim 8, wherein the nucleic acid barcode construct is linked to the DNAFile by a high affinity, non-covalent bond interaction between a biotin molecule linked to the 5′ and/or the 3′ end of the nucleic acid barcode construct and a molecule that binds to biotin, said biotin binding molecule being linked to the DNAFile.

10. A method of retrieving digital data stored in DNA, said method comprising
providing the library of origami folded DNAFiles according to claim 1; denaturing a folded origami DNAFile of said library to at least partially disrupt the
hybridized duplex between the single stranded staple oligonucleotides, data oligonucleotides and the DNA scaffold;
conducting PCR amplification on select nucleic acid sequences of said denatured DNA scaffold and data oligonucleotides to produce amplicons;
reannealing the staple oligonucleotides and data oligonucleotides with the DNA scaffold to reconstitute the folded origami DNAFile;
separating the amplicons from the reconstituted folded origami DNAFile; returning the reconstituted folded origami DNAFile to the library; and sequencing the amplicons to retrieve digital data encoded by the DNAFile.

11. The method of claim 10 wherein said denaturing step completely releases all staple oligonucleotides and data oligonucleotides as free single stranded nucleic acids.

12. The method of claim 10 wherein the amplicons are separated from the reconstituted folded origami DNAFiles
i) via gel electrophoresis; or
ii) via size exclusion chromatography.

13. The method of claim 10 further comprising the step of confirming the correct size and shape of the reconstituted folded origami DNA scaffold prior to returning the reconstituted folded origami DNA scaffold to the library.

14. The method of claim 13 further comprising the step of selecting one or more individual origami folded DNAFiles from the other origami folded DNAFiles of said library and conducting the denaturing step only on the selected origami folded DNAFiles.

15. The method of claim 14 wherein the one or more individual origami folded DNAFiles are selected based on selective binding of individual origami folded DNAFiles to a complementary oligonucleotide immobilized on a solid surface, or to a complementary oligonucleotide bound to a magnetic or fluorescently labelled nanoparticle.

16. A method of storing digital information using DNA as the storage medium, said method comprising the steps:
providing a single stranded DNA scaffold; and
providing a plurality of single stranded staple oligonucleotides that each bind through complementary base pairing with two non-contiguous nucleic acid sequences of the DNA scaffold, wherein said staple oligonucleotides cause the DNA scaffold to fold into a two or three dimensional shape having a plurality of external surfaces; mixing said DNA scaffold and said staple oligonucleotides under conditions that allow sequence specific hybridization of the staple oligonucleotides to the DNA scaffold and folding of the DNA scaffold; and
hybridizing a plurality of data oligonucleotides to an external surface of said plurality of external surfaces to store digital information using said data oligonucleotides as the storage medium, wherein said data oligonucleotides comprise a sequence complementary to a segment of said single stranded DNA scaffold, a nucleic acid sequence that encodes digital information, a first primer binding sequence and a second primer binding sequence, wherein the first primer binding sequence is locate 5′ to the digital information encoding nucleic acid sequence, and the second primer binding sequence is locate 3′ to the digital information encoding nucleic acid sequence.

17. The method of claim 16 wherein said staple oligonucleotides cause the single stranded DNA scaffold to fold into a multi-layered sheet conformation having a top surface and a bottom surface.


SEQUENCE LISTING (1003 sequences)

SEQ ID NO: 1 - DNA, 10 bp, synthetic construct
  source 1..10: mol_type = other DNA
gctacataat

SEQ ID NO: 2 - DNA, 10 bp, synthetic construct
  source 1..10: mol_type = other DNA
atgttacaca

SEQ ID NO: 3 - DNA, 10 bp, synthetic construct
  source 1..10: mol_type = other DNA
tggggcccaa

SEQ ID NO: 4 - DNA, 10 bp, synthetic construct
  source 1..10: mol_type = other DNA
tagtttatcc

SEQ ID NO: 5 - DNA, 10 bp, synthetic construct
  source 1..10: mol_type = other DNA
accccgtctt

SEQ ID NO: 6 - DNA, 10 bp, synthetic construct
  source 1..10: mol_type = other DNA
ccggccatca

SEQ ID NO: 7 - DNA, 10 bp, synthetic construct
  source 1..10: mol_type = other DNA
gagcttgctc

SEQ ID NO: 8 - DNA, 10 bp, synthetic construct
  source 1..10: mol_type = other DNA
acgttctata

SEQ ID NO: 9 - DNA, 10 bp, synthetic construct
  source 1..10: mol_type = other DNA
tacagcaaaa

SEQ ID NO: 10 - DNA, 10 bp, synthetic construct
  source 1..10: mol_type = other DNA
gttaggtggt

SEQ ID NO: 11 - DNA, 10 bp, synthetic construct
  source 1..10: mol_type = other DNA
ggagaccgac

SEQ ID NO: 12 - DNA, 10 bp, synthetic construct
  source 1..10: mol_type = other DNA
tggccccttg

SEQ ID NO: 13 - DNA, 10 bp, synthetic construct
  source 1..10: mol_type = other DNA
tggccgtaag

SEQ ID NO: 14 - DNA, 10 bp, synthetic construct
  source 1..10: mol_type = other DNA
cgttcgtcaa

SEQ ID NO: 15 - DNA, 10 bp, synthetic construct
  source 1..10: mol_type = other DNA
cggacgtgga

SEQ ID NO: 16 - DNA, 10 bp, synthetic construct
  source 1..10: mol_type = other DNA
agagggggca

SEQ ID NO: 17 - DNA, 10 bp, synthetic construct
  source 1..10: mol_type = other DNA
gttcaggtcg

SEQ ID NO: 18 - DNA, 10 bp, synthetic construct
  source 1..10: mol_type = other DNA
ctcgcaagag

SEQ ID NO: 19 - DNA, 10 bp, synthetic construct
  source 1..10: mol_type = other DNA
gcaacgactt

SEQ ID NO: 20 - DNA, 10 bp, synthetic construct
  source 1..10: mol_type = other DNA
gccatccatc

SEQ ID NO: 21 - DNA, 10 bp, synthetic construct
  source 1..10: mol_type = other DNA
ttccgagcag

SEQ ID NO: 22 - DNA, 10 bp, synthetic construct
  source 1..10: mol_type = other DNA
cttctggaca

SEQ ID NO: 23 - DNA, 10 bp, synthetic construct
  source 1..10: mol_type = other DNA
aacattagac

SEQ ID NO: 24 - DNA, 10 bp, synthetic construct
  source 1..10: mol_type = other DNA
aagcaatagt

SEQ ID NO: 25 - DNA, 10 bp, synthetic construct
  source 1..10: mol_type = other DNA
agggtaagac

SEQ ID NO: 26 - DNA, 10 bp, synthetic construct
  source 1..10: mol_type = other DNA
cgttgtcttg

SEQ ID NO: 27 - DNA, 10 bp, synthetic construct
  source 1..10: mol_type = other DNA
tttccccgcc

SEQ ID NO: 28 - DNA, 10 bp, synthetic construct
  source 1..10: mol_type = other DNA
cgaatggatc

SEQ ID NO: 29 - DNA, 10 bp, synthetic construct
  source 1..10: mol_type = other DNA
catcacttgc

SEQ ID NO: 30 - DNA, 10 bp, synthetic construct
  source 1..10: mol_type = other DNA
ctctcgcact

SEQ ID NO: 31 - DNA, 10 bp, synthetic construct
  source 1..10: mol_type = other DNA
gttcacgtgc

SEQ ID NO: 32 - DNA, 10 bp, synthetic construct
  source 1..10: mol_type = other DNA
aataagcctg

SEQ ID NO: 33 - DNA, 10 bp, synthetic construct
  source 1..10: mol_type = other DNA
gttaacaatt

SEQ ID NO: 34 - DNA, 10 bp, synthetic construct
  source 1..10: mol_type = other DNA
attcagatcc

SEQ ID NO: 35 - DNA, 10 bp, synthetic construct
  source 1..10: mol_type = other DNA
cctgctgatt

SEQ ID NO: 36 - DNA, 10 bp, synthetic construct
  source 1..10: mol_type = other DNA
cttggtcata

SEQ ID NO: 37 - DNA, 10 bp, synthetic construct
  source 1..10: mol_type = other DNA
tcttcctgtt

SEQ ID NO: 38 - DNA, 10 bp, synthetic construct
  source 1..10: mol_type = other DNA
actgccatgg

SEQ ID NO: 39 - DNA, 10 bp, synthetic construct
  source 1..10: mol_type = other DNA
catgtatagt

SEQ ID NO: 40 - DNA, 10 bp, synthetic construct
  source 1..10: mol_type = other DNA
ggtagcggca

SEQ ID NO: 41 - DNA, 10 bp, synthetic construct
  source 1..10: mol_type = other DNA
tcactctaac

SEQ ID NO: 42 - DNA, 10 bp, synthetic construct
  source 1..10: mol_type = other DNA
aaggtgcacc

SEQ ID NO: 43 - DNA, 10 bp, synthetic construct
  source 1..10: mol_type = other DNA
aatgctcgtt

SEQ ID NO: 44 - DNA, 10 bp, synthetic construct
  source 1..10: mol_type = other DNA
tgtctagaaa

SEQ ID NO: 45 - DNA, 10 bp, synthetic construct
  source 1..10: mol_type = other DNA
ctgcctgcct

SEQ ID NO: 46 - DNA, 10 bp, synthetic construct
  source 1..10: mol_type = other DNA
actataaaag

SEQ ID NO: 47 - DNA, 10 bp, synthetic construct
  source 1..10: mol_type = other DNA
tagtatcgag

SEQ ID NO: 48 - DNA, 10 bp, synthetic construct
  source 1..10: mol_type = other DNA
atcgcagtcc

SEQ ID NO: 49 - DNA, 10 bp, synthetic construct
  source 1..10: mol_type = other DNA
tcatcagaac

SEQ ID NO: 50 - DNA, 10 bp, synthetic construct
  source 1..10: mol_type = other DNA
tcctagacgc

SEQ ID NO: 51 - DNA, 10 bp, synthetic construct
  source 1..10: mol_type = other DNA
gccgggcggg

SEQ ID NO: 52 - DNA, 10 bp, synthetic construct
  source 1..10: mol_type = other DNA
gcccagaaga

SEQ ID NO: 53 - DNA, 10 bp, synthetic construct
  source 1..10: mol_type = other DNA
cttagagctg

SEQ ID NO: 54 - DNA, 10 bp, synthetic construct
  source 1..10: mol_type = other DNA
gtctgcgctt

SEQ ID NO: 55 - DNA, 10 bp, synthetic construct
  source 1..10: mol_type = other DNA
cgccgtcctt

SEQ ID NO: 56 - DNA, 10 bp, synthetic construct
  source 1..10: mol_type = other DNA
tttatctgct

SEQ ID NO: 57 - DNA, 10 bp, synthetic construct
  source 1..10: mol_type = other DNA
tgcttcggag

SEQ ID NO: 58 - DNA, 10 bp, synthetic construct
  source 1..10: mol_type = other DNA
ggggagaatg

SEQ ID NO: 59 - DNA, 10 bp, synthetic construct
  source 1..10: mol_type = other DNA
gtggtaagtg

SEQ ID NO: 60 - DNA, 10 bp, synthetic construct
  source 1..10: mol_type = other DNA
gaaattagta

SEQ ID NO: 61 - DNA, 10 bp, synthetic construct
  source 1..10: mol_type = other DNA
gctatcctaa

SEQ ID NO: 62 - DNA, 10 bp, synthetic construct
  source 1..10: mol_type = other DNA
atctgtacga

SEQ ID NO: 63 - DNA, 10 bp, synthetic construct
  source 1..10: mol_type = other DNA
agttcggggc

SEQ ID NO: 64 - DNA, 10 bp, synthetic construct
  source 1..10: mol_type = other DNA
cgagtctgtc

SEQ ID NO: 65 - DNA, 10 bp, synthetic construct
  source 1..10: mol_type = other DNA
atcctacgca

SEQ ID NO: 66 - DNA, 10 bp, synthetic construct
  source 1..10: mol_type = other DNA
atggtggata

SEQ ID NO: 67 - DNA, 10 bp, synthetic construct
  source 1..10: mol_type = other DNA
cctctaacta

SEQ ID NO: 68 - DNA, 10 bp, synthetic construct
  source 1..10: mol_type = other DNA
atagctgcac

SEQ ID NO: 69 - DNA, 10 bp, synthetic construct
  source 1..10: mol_type = other DNA
gacagaattt

SEQ ID NO: 70 - DNA, 10 bp, synthetic construct
  source 1..10: mol_type = other DNA
caattggcat

SEQ ID NO: 71 - DNA, 10 bp, synthetic construct
  source 1..10: mol_type = other DNA
tctagtagac

SEQ ID NO: 72 - DNA, 10 bp, synthetic construct
  source 1..10: mol_type = other DNA
ttattcatgg

SEQ ID NO: 73 - DNA, 10 bp, synthetic construct
  source 1..10: mol_type = other DNA
ttggcaaccg

SEQ ID NO: 74 - DNA, 10 bp, synthetic construct
  source 1..10: mol_type = other DNA
cataatacat

SEQ ID NO: 75 - DNA, 10 bp, synthetic construct
  source 1..10: mol_type = other DNA
acagactcac

SEQ ID NO: 76 - DNA, 10 bp, synthetic construct
  source 1..10: mol_type = other DNA
gcgatgctgc

SEQ ID NO: 77 - DNA, 10 bp, synthetic construct
  source 1..10: mol_type = other DNA
catctttgcc

SEQ ID NO: 78 - DNA, 10 bp, synthetic construct
  source 1..10: mol_type = other DNA
gtgactccag

SEQ ID NO: 79 - DNA, 10 bp, synthetic construct
  source 1..10: mol_type = other DNA
ggacgagtct

SEQ ID NO: 80 - DNA, 10 bp, synthetic construct
  source 1..10: mol_type = other DNA
tagtggcgtg

SEQ ID NO: 81 - DNA, 10 bp, synthetic construct
  source 1..10: mol_type = other DNA
aacgcagctt

SEQ ID NO: 82 - DNA, 10 bp, synthetic construct
  source 1..10: mol_type = other DNA
agaacaggtg

SEQ ID NO: 83 - DNA, 10 bp, synthetic construct
  source 1..10: mol_type = other DNA
aggctatgtt

SEQ ID NO: 84 - DNA, 10 bp, synthetic construct
  source 1..10: mol_type = other DNA
cctggatctt

SEQ ID NO: 85 - DNA, 10 bp, synthetic construct
  source 1..10: mol_type = other DNA
ctagccggcc

SEQ ID NO: 86 - DNA, 10 bp, synthetic construct
  source 1..10: mol_type = other DNA
accagttatc

SEQ ID NO: 87 - DNA, 10 bp, synthetic construct
  source 1..10: mol_type = other DNA
acgttatagc

SEQ ID NO: 88 - DNA, 10 bp, synthetic construct
  source 1..10: mol_type = other DNA
tcgagtttga

SEQ ID NO: 89 - DNA, 10 bp, synthetic construct
  source 1..10: mol_type = other DNA
tgaagcgagc

SEQ ID NO: 90 - DNA, 10 bp, synthetic construct
  source 1..10: mol_type = other DNA
gactggcgaa

SEQ ID NO: 91 - DNA, 10 bp, synthetic construct
  source 1..10: mol_type = other DNA
gatggaccta

SEQ ID NO: 92 - DNA, 10 bp, synthetic construct
  source 1..10: mol_type = other DNA
gtccacaacg

SEQ ID NO: 93 - DNA, 10 bp, synthetic construct
  source 1..10: mol_type = other DNA
cctccccaga

SEQ ID NO: 94 - DNA, 10 bp, synthetic construct
  source 1..10: mol_type = other DNA
ttatgacgcc

SEQ ID NO: 95 - DNA, 10 bp, synthetic construct
  source 1..10: mol_type = other DNA
cttgatccgt

SEQ ID NO: 96 - DNA, 10 bp, synthetic construct
  source 1..10: mol_type = other DNA
aatgcgcaat

SEQ ID NO: 97 - DNA, 10 bp, synthetic construct
  source 1..10: mol_type = other DNA
gtacccctca

SEQ ID NO: 98 - DNA, 10 bp, synthetic construct
  source 1..10: mol_type = other DNA
cgacagctcg

SEQ ID NO: 99 - DNA, 10 bp, synthetic construct
  source 1..10: mol_type = other DNA
tgacctggct

SEQ ID NO: 100 - DNA, 10 bp, synthetic construct
  source 1..10: mol_type = other DNA
ttcatagccc

SEQ ID NO: 101 - DNA, 10 bp, synthetic construct
  source 1..10: mol_type = other DNA
cccaagagaa

SEQ ID NO: 102 - DNA, 10 bp, synthetic construct
  source 1..10: mol_type = other DNA
aaacgaagta

SEQ ID NO: 103 - DNA, 10 bp, synthetic construct
  source 1..10: mol_type = other DNA
gacgtttaca

SEQ ID NO: 104 - DNA, 10 bp, synthetic construct
  source 1..10: mol_type = other DNA
gatcgatttg

SEQ ID NO: 105 - DNA, 10 bp, synthetic construct
  source 1..10: mol_type = other DNA
cactgtcacc

SEQ ID NO: 106 - DNA, 10 bp, synthetic construct
  source 1..10: mol_type = other DNA
tgtgagagtt

SEQ ID NO: 107 - DNA, 10 bp, synthetic construct
  source 1..10: mol_type = other DNA
gacgtaacct

SEQ ID NO: 108 - DNA, 10 bp, synthetic construct
  source 1..10: mol_type = other DNA
cagactctgc

SEQ ID NO: 109 - DNA, 10 bp, synthetic construct
  source 1..10: mol_type = other DNA
tatgccaata

SEQ ID NO: 110 - DNA, 10 bp, synthetic construct
  source 1..10: mol_type = other DNA
acaggtgatg

SEQ ID NO: 111 - DNA, 10 bp, synthetic construct
  source 1..10: mol_type = other DNA
gtcatcgcgt

SEQ ID NO: 112 - DNA, 10 bp, synthetic construct
  source 1..10: mol_type = other DNA
tcttataaac

SEQ ID NO: 113 - DNA, 10 bp, synthetic construct
  source 1..10: mol_type = other DNA
gtgtagactg

SEQ ID NO: 114 - DNA, 10 bp, synthetic construct
  source 1..10: mol_type = other DNA
aaacaaccgg

SEQ ID NO: 115 - DNA, 10 bp, synthetic construct
  source 1..10: mol_type = other DNA
atcctgtacc

SEQ ID NO: 116 - DNA, 10 bp, synthetic construct
  source 1..10: mol_type = other DNA
ttataagaat

SEQ ID NO: 117 - DNA, 10 bp, synthetic construct
  source 1..10: mol_type = other DNA
ataagtaggc

SEQ ID NO: 118 - DNA, 10 bp, synthetic construct
  source 1..10: mol_type = other DNA
tctcgtaagg

SEQ ID NO: 119 - DNA, 10 bp, synthetic construct
  source 1..10: mol_type = other DNA
gatccgccgc

SEQ ID NO: 120 - DNA, 10 bp, synthetic construct
  source 1..10: mol_type = other DNA
tgtcaggttt

SEQ ID NO: 121 - DNA, 10 bp, synthetic construct
  source 1..10: mol_type = other DNA
tccgaagccc

SEQ ID NO: 122 - DNA, 10 bp, synthetic construct
  source 1..10: mol_type = other DNA
tccatgtcca

SEQ ID NO: 123 - DNA, 10 bp, synthetic construct
  source 1..10: mol_type = other DNA
gtgatggtac

SEQ ID NO: 124 - DNA, 10 bp, synthetic construct
  source 1..10: mol_type = other DNA
ctccacatac

SEQ ID NO: 125 - DNA, 10 bp, synthetic construct
  source 1..10: mol_type = other DNA
ttcggatgag

SEQ ID NO: 126 - DNA, 10 bp, synthetic construct
  source 1..10: mol_type = other DNA
acgacatcgc

SEQ ID NO: 127 - DNA, 10 bp, synthetic construct
  source 1..10: mol_type = other DNA
gagatgcaca

SEQ ID NO: 128 - DNA, 10 bp, synthetic construct
  source 1..10: mol_type = other DNA
tttgtatggc

SEQ ID NO: 129 - DNA, 10 bp, synthetic construct
  source 1..10: mol_type = other DNA
cttttctaga

SEQ ID NO: 130 - DNA, 10 bp, synthetic construct
  source 1..10: mol_type = other DNA
agtctaatca

SEQ ID NO: 131 - DNA, 10 bp, synthetic construct
  source 1..10: mol_type = other DNA
gacttagcca

SEQ ID NO: 132 - DNA, 10 bp, synthetic construct
  source 1..10: mol_type = other DNA
tatcacagta

SEQ ID NO: 133 - DNA, 10 bp, synthetic construct
  source 1..10: mol_type = other DNA
aagctcgagt

SEQ ID NO: 134 - DNA, 10 bp, synthetic construct
  source 1..10: mol_type = other DNA
tgttacgaca

SEQ ID NO: 135 - DNA, 10 bp, synthetic construct
  source 1..10: mol_type = other DNA
aaggatagtc

SEQ ID NO: 136 - DNA, 10 bp, synthetic construct
  source 1..10: mol_type = other DNA
gcacttagcc

SEQ ID NO: 137 - DNA, 10 bp, synthetic construct
  source 1..10: mol_type = other DNA
gagggatccg

SEQ ID NO: 138 - DNA, 10 bp, synthetic construct
  source 1..10: mol_type = other DNA
attctagaag

SEQ ID NO: 139 - DNA, 10 bp, synthetic construct
  source 1..10: mol_type = other DNA
gataactgat

SEQ ID NO: 140 - DNA, 10 bp, synthetic construct
  source 1..10: mol_type = other DNA
atctgactgt

SEQ ID NO: 141 - DNA, 10 bp, synthetic construct
  source 1..10: mol_type = other DNA
caaagcgaac

SEQ ID NO: 142 - DNA, 10 bp, synthetic construct
  source 1..10: mol_type = other DNA
gaaattgcga

SEQ ID NO: 143 - DNA, 10 bp, synthetic construct
  source 1..10: mol_type = other DNA
gggtccagtc

SEQ ID NO: 144 - DNA, 10 bp, synthetic construct
  source 1..10: mol_type = other DNA
atcaggtagc

SEQ ID NO: 145 - DNA, 10 bp, synthetic construct
  source 1..10: mol_type = other DNA
gaaaggtcct

SEQ ID NO: 146 - DNA, 10 bp, synthetic construct
  source 1..10: mol_type = other DNA
ggctaccaca

SEQ ID NO: 147 - DNA, 10 bp, synthetic construct
  source 1..10: mol_type = other DNA
ttattgctga

SEQ ID NO: 148 - DNA, 10 bp, synthetic construct
  source 1..10: mol_type = other DNA
cgccgcgttt

SEQ ID NO: 149 - DNA, 10 bp, synthetic construct
  source 1..10: mol_type = other DNA
ttttcaaaag

SEQ ID NO: 150 - DNA, 10 bp, synthetic construct
  source 1..10: mol_type = other DNA
ctgggctaaa

SEQ ID NO: 151 - DNA, 10 bp, synthetic construct
  source 1..10: mol_type = other DNA
cccgatgaga

SEQ ID NO: 152 - DNA, 10 bp, synthetic construct
  source 1..10: mol_type = other DNA
tgggaaatat

SEQ ID NO: 153 - DNA, 10 bp, synthetic construct
  source 1..10: mol_type = other DNA
gtacgagcgg

SEQ ID NO: 154 - DNA, 10 bp, synthetic construct
  source 1..10: mol_type = other DNA
gcgtgcagct

SEQ ID NO: 155 - DNA, 10 bp, synthetic construct
  source 1..10: mol_type = other DNA
agtctgcgga

SEQ ID NO: 156 - DNA, 10 bp, synthetic construct
  source 1..10: mol_type = other DNA
taactattta

SEQ ID NO: 157 - DNA, 10 bp, synthetic construct
  source 1..10: mol_type = other DNA
gagttgccgg

SEQ ID NO: 158 - DNA, 10 bp, synthetic construct
  source 1..10: mol_type = other DNA
cagcccggcg

SEQ ID NO: 159 - DNA, 10 bp, synthetic construct
  source 1..10: mol_type = other DNA
tcacctacat

SEQ ID NO: 160 - DNA, 10 bp, synthetic construct
  source 1..10: mol_type = other DNA
agtggctaac

SEQ ID NO: 161 - DNA, 10 bp, synthetic construct
  source 1..10: mol_type = other DNA
agaatgtgag

SEQ ID NO: 162 - DNA, 10 bp, synthetic construct
  source 1..10: mol_type = other DNA
tagtttcgca

SEQ ID NO: 163 - DNA, 10 bp, synthetic construct
  source 1..10: mol_type = other DNA
cttcatttct

SEQ ID NO: 164 - DNA, 10 bp, synthetic construct
  source 1..10: mol_type = other DNA
gccatgatat

SEQ ID NO: 165 - DNA, 10 bp, synthetic construct
  source 1..10: mol_type = other DNA
acggcaaatc

SEQ ID NO: 166 - DNA, 10 bp, synthetic construct
  source 1..10: mol_type = other DNA
atcgatagta

SEQ ID NO: 167 - DNA, 10 bp, synthetic construct
  source 1..10: mol_type = other DNA
cctaaaggca

SEQ ID NO: 168 - DNA, 10 bp, synthetic construct
  source 1..10: mol_type = other DNA
tacgagcggt

SEQ ID NO: 169 - DNA, 10 bp, synthetic construct
  source 1..10: mol_type = other DNA
tttgtcgtcg

SEQ ID NO: 170 - DNA, 10 bp, synthetic construct
  source 1..10: mol_type = other DNA
tacaagcttg

SEQ ID NO: 171 - DNA, 10 bp, synthetic construct
  source 1..10: mol_type = other DNA
gaccaacacg

SEQ ID NO: 172 - DNA, 10 bp, synthetic construct
  source 1..10: mol_type = other DNA
gaacgacgaa

SEQ ID NO: 173 - DNA, 10 bp, synthetic construct
  source 1..10: mol_type = other DNA
tcggaacgca

SEQ ID NO: 174 - DNA, 10 bp, synthetic construct
  source 1..10: mol_type = other DNA
atccggtggt

SEQ ID NO: 175 - DNA, 10 bp, synthetic construct
  source 1..10: mol_type = other DNA
taaaacgtag

SEQ ID NO: 176 - DNA, 10 bp, synthetic construct
  source 1..10: mol_type = other DNA
tatgtgagcc

SEQ ID NO: 177 - DNA, 10 bp, synthetic construct
  source 1..10: mol_type = other DNA
gaggcatcga

SEQ ID NO: 178 - DNA, 10 bp, synthetic construct
  source 1..10: mol_type = other DNA
gaatgggtgg

SEQ ID NO: 179 - DNA, 10 bp, synthetic construct
  source 1..10: mol_type = other DNA
aacgacacaa

SEQ ID NO: 180 - DNA, 10 bp, synthetic construct
  source 1..10: mol_type = other DNA
gtacgatgca

SEQ ID NO: 181 - DNA, 10 bp, synthetic construct
  source 1..10: mol_type = other DNA
agaaggcgcc

SEQ ID NO: 182 - DNA, 10 bp, synthetic construct
  source 1..10: mol_type = other DNA
ccgcaatgga

SEQ ID NO: 183 - DNA, 10 bp, synthetic construct
  source 1..10: mol_type = other DNA
tacggatttt

SEQ ID NO: 184 - DNA, 10 bp, synthetic construct
  source 1..10: mol_type = other DNA
gtcgttagct

SEQ ID NO: 185 - DNA, 10 bp, synthetic construct
  source 1..10: mol_type = other DNA
ggactagggc

SEQ ID NO: 186 - DNA, 10 bp, synthetic construct
  source 1..10: mol_type = other DNA
attggtattc

SEQ ID NO: 187 - DNA, 10 bp, synthetic construct
  source 1..10: mol_type = other DNA
atcccagaga

SEQ ID NO: 188 - DNA, 10 bp, synthetic construct
  source 1..10: mol_type = other DNA
gtcccagctc

SEQ ID NO: 189 - DNA, 10 bp, synthetic construct
  source 1..10: mol_type = other DNA
cacgaggaat

SEQ ID NO: 190 - DNA, 10 bp, synthetic construct
  source 1..10: mol_type = other DNA
tacaattgca

SEQ ID NO: 191 - DNA, 10 bp, synthetic construct
  source 1..10: mol_type = other DNA
attcctgaat

SEQ ID NO: 192 - DNA, 10 bp, synthetic construct
  source 1..10: mol_type = other DNA
tagcgaggcg

SEQ ID NO: 193 - DNA, 10 bp, synthetic construct
  source 1..10: mol_type = other DNA
ctggatgggc

SEQ ID NO: 194 - DNA, 10 bp, synthetic construct
  source 1..10: mol_type = other DNA
gcgacggcca

SEQ ID NO: 195 - DNA, 10 bp, synthetic construct
  source 1..10: mol_type = other DNA
acctgcacaa

SEQ ID NO: 196 - DNA, 10 bp, synthetic construct
  source 1..10: mol_type = other DNA
catgacagac

SEQ ID NO: 197 - DNA, 10 bp, synthetic construct
  source 1..10: mol_type = other DNA
ttaccaacgt

SEQ ID NO: 198 - DNA, 10 bp, synthetic construct
  source 1..10: mol_type = other DNA
caggtgtgtg

SEQ ID NO: 199 - DNA, 10 bp, synthetic construct
  source 1..10: mol_type = other DNA
cgagggacgg

SEQ ID NO: 200 - DNA, 10 bp, synthetic construct
  source 1..10: mol_type = other DNA
cgtctcggta

SEQ ID NO: 201 - DNA, 10 bp, synthetic construct
  source 1..10: mol_type = other DNA
taagctatct

SEQ ID NO: 202 - DNA, 10 bp, synthetic construct
  source 1..10: mol_type = other DNA
tactccccta

SEQ ID NO: 203 - DNA, 10 bp, synthetic construct
  source 1..10: mol_type = other DNA
ttatattcat

SEQ ID NO: 204 - DNA, 10 bp, synthetic construct
  source 1..10: mol_type = other DNA
agcgatctgc

SEQ ID NO: 205 - DNA, 10 bp, synthetic construct
  source 1..10: mol_type = other DNA
tcttctgatc

SEQ ID NO: 206 - DNA, 10 bp, synthetic construct
  source 1..10: mol_type = other DNA
atagttccca

SEQ ID NO: 207 - DNA, 10 bp, synthetic construct
  source 1..10: mol_type = other DNA
tttacgggtg

SEQ ID NO: 208 - DNA, 10 bp, synthetic construct
  source 1..10: mol_type = other DNA
gtgtcccctg

SEQ ID NO: 209 - DNA, 10 bp, synthetic construct
  source 1..10: mol_type = other DNA
gcgggggtcg

SEQ ID NO: 210 - DNA, 10 bp, synthetic construct
  source 1..10: mol_type = other DNA
cattgatcta

SEQ ID NO: 211 - DNA, 10 bp, synthetic construct
  source 1..10: mol_type = other DNA
agggacggtg

SEQ ID NO: 212 - DNA, 10 bp, synthetic construct
  source 1..10: mol_type = other DNA
cagttacttt

SEQ ID NO: 213 - DNA, 10 bp, synthetic construct
  source 1..10: mol_type = other DNA
ccatacttcc

SEQ ID NO: 214 - DNA, 10 bp, synthetic construct
  source 1..10: mol_type = other DNA
atcagaatta

SEQ ID NO: 215 - DNA, 10 bp, synthetic construct
  source 1..10: mol_type = other DNA
aaactaggca

SEQ ID NO: 216 - DNA, 10 bp, synthetic construct
  source 1..10: mol_type = other DNA
aatgtcgttg

SEQ ID NO: 217 - DNA, 10 bp, synthetic construct
  source 1..10: mol_type = other DNA
cacatgggtc

SEQ ID NO: 218 - DNA, 10 bp, synthetic construct
  source 1..10: mol_type = other DNA
ggtcgctggt

SEQ ID NO: 219 - DNA, 10 bp, synthetic construct
  source 1..10: mol_type = other DNA
actgtattac

SEQ ID NO: 220 - DNA, 10 bp, synthetic construct
  source 1..10: mol_type = other DNA
ccgagacgcg

SEQ ID NO: 221 - DNA, 10 bp, synthetic construct
  source 1..10: mol_type = other DNA
actccaaccc

SEQ ID NO: 222 - DNA, 10 bp, synthetic construct
  source 1..10: mol_type = other DNA
atattacaag

SEQ ID NO: 223 - DNA, 10 bp, synthetic construct
  source 1..10: mol_type = other DNA
ccatggatag

SEQ ID NO: 224 - DNA, 10 bp, synthetic construct
  source 1..10: mol_type = other DNA
ccgtctcaat

SEQ ID NO: 225 - DNA, 10 bp, synthetic construct
  source 1..10: mol_type = other DNA
gatcgtcggg

SEQ ID NO: 226 - DNA, 10 bp, synthetic construct
  source 1..10: mol_type = other DNA
tcttgttttg

SEQ ID NO: 227 - DNA, 10 bp, synthetic construct
  source 1..10: mol_type = other DNA
aatattgctc

SEQ ID NO: 228 - DNA, 10 bp, synthetic construct
  source 1..10: mol_type = other DNA
aacgtcgtct

SEQ ID NO: 229 - DNA, 10 bp, synthetic construct
  source 1..10: mol_type = other DNA
aatatttttg

SEQ ID NO: 230 - DNA, 10 bp, synthetic construct
  source 1..10: mol_type = other DNA
cgtaacgtgc

SEQ ID NO: 231 - DNA, 10 bp, synthetic construct
  source 1..10: mol_type = other DNA
gcgtggttat

SEQ ID NO: 232 - DNA, 10 bp, synthetic construct
  source 1..10: mol_type = other DNA
caaaacatta

SEQ ID NO: 233 - DNA, 10 bp, synthetic construct
  source 1..10: mol_type = other DNA
cgtatcctga

SEQ ID NO: 234 - DNA, 10 bp, synthetic construct
  source 1..10: mol_type = other DNA
tcgcttacaa

SEQ ID NO: 235 - DNA, 10 bp, synthetic construct
  source 1..10: mol_type = other DNA
tccattgtgt

SEQ ID NO: 236 - DNA, 10 bp, synthetic construct
  source 1..10: mol_type = other DNA
gcccccattc

SEQ ID NO: 237 - DNA, 10 bp, synthetic construct
  source 1..10: mol_type = other DNA
tgacgtctat

SEQ ID NO: 238 - DNA, 10 bp, synthetic construct
  source 1..10: mol_type = other DNA
tgggccgagg

SEQ ID NO: 239 - DNA, 10 bp, synthetic construct
  source 1..10: mol_type = other DNA
aagtgtcaag

SEQ ID NO: 240 - DNA, 10 bp, synthetic construct
  source 1..10: mol_type = other DNA
gacagtagag

SEQ ID NO: 241 - DNA, 10 bp, synthetic construct
  source 1..10: mol_type = other DNA
cgcagccatc

SEQ ID NO: 242 - DNA, 10 bp, synthetic construct
  source 1..10: mol_type = other DNA
gaggcagaac

SEQ ID NO: 243 - DNA, 10 bp, synthetic construct
  source 1..10: mol_type = other DNA
gttgaaattg

SEQ ID NO: 244 - DNA, 10 bp, synthetic construct
  source 1..10: mol_type = other DNA
atctgataaa

SEQ ID NO: 245 - DNA, 10 bp, synthetic construct
  source 1..10: mol_type = other DNA
agctgtctct

SEQ ID NO: 246 - DNA, 10 bp, synthetic construct
  source 1..10: mol_type = other DNA
ttttaggtta

SEQ ID NO: 247 - DNA, 10 bp, synthetic construct
  source 1..10: mol_type = other DNA
tatctgtccg

SEQ ID NO: 248 - DNA, 10 bp, synthetic construct
  source 1..10: mol_type = other DNA
aaaacatatg

SEQ ID NO: 249 - DNA, 10 bp, synthetic construct
  source 1..10: mol_type = other DNA
gtaaagaaga

SEQ ID NO: 250 - DNA, 10 bp, synthetic construct
  source 1..10: mol_type = other DNA
tcgacgtgca

SEQ ID NO: 251 - DNA, 10 bp, synthetic construct
  source 1..10: mol_type = other DNA
tagatcttaa

SEQ ID NO: 252 - DNA, 10 bp, synthetic construct
  source 1..10: mol_type = other DNA
cactggtcac

SEQ ID NO: 253 - DNA, 10 bp, synthetic construct
  source 1..10: mol_type = other DNA
attctgatgt

SEQ ID NO: 254 - DNA, 10 bp, synthetic construct
  source 1..10: mol_type = other DNA
atggccctga

SEQ ID NO: 255 - DNA, 10 bp, synthetic construct
  source 1..10: mol_type = other DNA
ggtgatgaga

SEQ ID NO: 256 - DNA, 10 bp, synthetic construct
  source 1..10: mol_type = other DNA
caccgtgggg

SEQ ID NO: 257 - DNA, 10 bp, synthetic construct
  source 1..10: mol_type = other DNA
gcttgctcgg

SEQ ID NO: 258 - DNA, 10 bp, synthetic construct
  source 1..10: mol_type = other DNA
ccagttgaac

SEQ ID NO: 259 - DNA, 10 bp, synthetic construct
  source 1..10: mol_type = other DNA
cgtctgtacc

SEQ ID NO: 260 - DNA, 10 bp, synthetic construct
  source 1..10: mol_type = other DNA
ccaacgcggc

SEQ ID NO: 261 - DNA, 10 bp, synthetic construct
  source 1..10: mol_type = other DNA
acgtgatcga

SEQ ID NO: 262 - DNA, 10 bp, synthetic construct
  source 1..10: mol_type = other DNA
ccatcgaatc

SEQ ID NO: 263 - DNA, 10 bp, synthetic construct
  source 1..10: mol_type = other DNA
cggtgtctgc

SEQ ID NO: 264 - DNA, 10 bp, synthetic construct
  source 1..10: mol_type = other DNA
aaaccacctc

SEQ ID NO: 265 - DNA, 10 bp, synthetic construct
  source 1..10: mol_type = other DNA
tcaatgttcc

SEQ ID NO: 266 - DNA, 10 bp, synthetic construct
  source 1..10: mol_type = other DNA
ttcgacatgt

SEQ ID NO: 267 - DNA, 10 bp, synthetic construct
  source 1..10: mol_type = other DNA
aggcacgata

SEQ ID NO: 268 - DNA, 10 bp, synthetic construct
  source 1..10: mol_type = other DNA
cacgagatca

SEQ ID NO: 269 - DNA, 10 bp, synthetic construct
  source 1..10: mol_type = other DNA
catgctgggg

SEQ ID NO: 270 - DNA, 10 bp, synthetic construct
  source 1..10: mol_type = other DNA
taccatggtt

SEQ ID NO: 271 - DNA, 10 bp, synthetic construct
  source 1..10: mol_type = other DNA
ttgcccatat

SEQ ID NO: 272 - DNA, 10 bp, synthetic construct
  source 1..10: mol_type = other DNA
tgcacattcg

SEQ ID NO: 273 - DNA, 10 bp, synthetic construct
  source 1..10: mol_type = other DNA
gttatgttgg

SEQ ID NO: 274 - DNA, 10 bp, synthetic construct
  source 1..10: mol_type = other DNA
tgagttatga

SEQ ID NO: 275 - DNA, 10 bp, synthetic construct
  source 1..10: mol_type = other DNA
gatggccccc

SEQ ID NO: 276 - DNA, 10 bp, synthetic construct
  source 1..10: mol_type = other DNA
gatgggttac

SEQ ID NO: 277 - DNA, 10 bp, synthetic construct
  source 1..10: mol_type = other DNA
agctacgttg

SEQ ID NO: 278 - DNA, 10 bp, synthetic construct
  source 1..10: mol_type = other DNA
accccatgca

SEQ ID NO: 279 - DNA, 10 bp, synthetic construct
  source 1..10: mol_type = other DNA
tactaccgtt

SEQ ID NO: 280 - DNA, 10 bp, synthetic construct
  source 1..10: mol_type = other DNA
tcgcttctac

SEQ ID NO: 281 - DNA, 10 bp, synthetic construct
  source 1..10: mol_type = other DNA
ctggcagtgc

SEQ ID NO: 282 - DNA, 10 bp, synthetic construct
  source 1..10: mol_type = other DNA
tctatatata

SEQ ID NO: 283 - DNA, 10 bp, synthetic construct
  source 1..10: mol_type = other DNA
ggattagttc

SEQ ID NO: 284 - DNA, 10 bp, synthetic construct
  source 1..10: mol_type = other DNA
gtgttacgct

SEQ ID NO: 285 - DNA, 10 bp, synthetic construct
  source 1..10: mol_type = other DNA
tcgactccgt

SEQ ID NO: 286 - DNA, 10 bp, synthetic construct
  source 1..10: mol_type = other DNA
ggtagcaggc

SEQ ID NO: 287 - DNA, 10 bp, synthetic construct
  source 1..10: mol_type = other DNA
tattggattc

SEQ ID NO: 288 - DNA, 10 bp, synthetic construct
  source 1..10: mol_type = other DNA
gttcgatcga

SEQ ID NO: 289 - DNA, 10 bp, synthetic construct
  source 1..10: mol_type = other DNA
atattaatat

SEQ ID NO: 290 - DNA, 10 bp, synthetic construct
  source 1..10: mol_type = other DNA
agaacgattg

SEQ ID NO: 291 - DNA, 10 bp, synthetic construct
  source 1..10: mol_type = other DNA
gtaaagtgta

SEQ ID NO: 292 - DNA, 10 bp, synthetic construct
  source 1..10: mol_type = other DNA
cccatgtgcc

SEQ ID NO: 293 - DNA, 10 bp, synthetic construct
  source 1..10: mol_type = other DNA
gtggcctcgc

SEQ ID NO: 294 - DNA, 10 bp, synthetic construct
  source 1..10: mol_type = other DNA
gacactagga

SEQ ID NO: 295 - DNA, 10 bp, synthetic construct
  source 1..10: mol_type = other DNA
atattctgac

SEQ ID NO: 296 - DNA, 10 bp, synthetic construct
  source 1..10: mol_type = other DNA
taagtagacg

SEQ ID NO: 297 - DNA, 10 bp, synthetic construct
  source 1..10: mol_type = other DNA
taacggtcta

SEQ ID NO: 298 - DNA, 10 bp, synthetic construct
  source 1..10: mol_type = other DNA
tagtttcatt

SEQ ID NO: 299 - DNA, 10 bp, synthetic construct
  source 1..10: mol_type = other DNA
ttggatccga

SEQ ID NO: 300 - DNA, 10 bp, synthetic construct
  source 1..10: mol_type = other DNA
cgtgacaacc

SEQ ID NO: 301 - DNA, 10 bp, synthetic construct
  source 1..10: mol_type = other DNA
cgcgctcaga

SEQ ID NO: 302 - DNA, 10 bp, synthetic construct
  source 1..10: mol_type = other DNA
cgttcttaat

SEQ ID NO: 303 - DNA, 10 bp, synthetic construct
  source 1..10: mol_type = other DNA
acaagagttt

SEQ ID NO: 304 - DNA, 10 bp, synthetic construct
  source 1..10: mol_type = other DNA
agggttatag

SEQ ID NO: 305 - DNA, 10 bp, synthetic construct
  source 1..10: mol_type = other DNA
accacgactc

SEQ ID NO: 306 - DNA, 10 bp, synthetic construct
  source 1..10: mol_type = other DNA
gtactcgggg

SEQ ID NO: 307 - DNA, 10 bp, synthetic construct
  source 1..10: mol_type = other DNA
acaaatatct

SEQ ID NO: 308 - DNA, 10 bp, synthetic construct
  source 1..10: mol_type = other DNA
gatcggggtg

SEQ ID NO: 309 - DNA, 10 bp, synthetic construct
  source 1..10: mol_type = other DNA
atgtaactcc

SEQ ID NO: 310 - DNA, 10 bp, synthetic construct
  source 1..10: mol_type = other DNA
atgaagaagc

SEQ ID NO: 311 - DNA, 10 bp, synthetic construct
  source 1..10: mol_type = other DNA
atgtattgtc

SEQ ID NO: 312 - DNA, 10 bp, synthetic construct
  source 1..10: mol_type = other DNA
tgcattggaa

SEQ ID NO: 313 - DNA, 10 bp, synthetic construct
  source 1..10: mol_type = other DNA
gcggacgatc

SEQ ID NO: 314 - DNA, 10 bp, synthetic construct
  source 1..10: mol_type = other DNA
ccgtacttga

SEQ ID NO: 315 - DNA, 10 bp, synthetic construct
  source 1..10: mol_type = other DNA
tttgcccccg

SEQ ID NO: 316 - DNA, 10 bp, synthetic construct
  source 1..10: mol_type = other DNA
acctcacgcg

SEQ ID NO: 317 - DNA, 10 bp, synthetic construct
  source 1..10: mol_type = other DNA
attaaggggc

SEQ ID NO: 318 - DNA, 10 bp, synthetic construct
  source 1..10: mol_type = other DNA
cgtggacatg

SEQ ID NO: 319 - DNA, 10 bp, synthetic construct
  source 1..10: mol_type = other DNA
ttagcccttc

SEQ ID NO: 320 - DNA, 10 bp, synthetic construct
  source 1..10: mol_type = other DNA
cgagagtttg

SEQ ID NO: 321 - DNA, 10 bp, synthetic construct
  source 1..10: mol_type = other DNA
tgcatcctct

SEQ ID NO: 322 - DNA, 10 bp, synthetic construct
  source 1..10: mol_type = other DNA
tgcgattccg

SEQ ID NO: 323 - DNA, 10 bp, synthetic construct
  source 1..10: mol_type = other DNA
ttattacgtt

SEQ ID NO: 324 - DNA, 10 bp, synthetic construct
  source 1..10: mol_type = other DNA
tgatgtggtt

SEQ ID NO: 325 - DNA, 10 bp, synthetic construct
  source 1..10: mol_type = other DNA
gggcgtcaat

SEQ ID NO: 326 - DNA, 10 bp, synthetic construct
  source 1..10: mol_type = other DNA
cccttgaaat

SEQ ID NO: 327 - DNA, 10 bp, synthetic construct
  source 1..10: mol_type = other DNA
tctttggggc

SEQ ID NO: 328 - DNA, 10 bp, synthetic construct
  source 1..10: mol_type = other DNA
accggcaggc

SEQ ID NO: 329 - DNA, 10 bp, synthetic construct
  source 1..10: mol_type = other DNA
gctaaaatct

SEQ ID NO: 330 - DNA, 10 bp, synthetic construct
  source 1..10: mol_type = other DNA
gccgttgacg

SEQ ID NO: 331 - DNA, 10 bp, synthetic construct
  source 1..10: mol_type = other DNA
ggagttgttg

SEQ ID NO: 332 - DNA, 10 bp, synthetic construct
  source 1..10: mol_type = other DNA
tacttgagaa

SEQ ID NO: 333 - DNA, 10 bp, synthetic construct
  source 1..10: mol_type = other DNA
cgggtgcgct

SEQ ID NO: 334 - DNA, 10 bp, synthetic construct
  source 1..10: mol_type = other DNA
aaaagcgtct

SEQ ID NO: 335 - DNA, 10 bp, synthetic construct
  source 1..10: mol_type = other DNA
gtaaagatag

SEQ ID NO: 336 - DNA, 10 bp, synthetic construct
  source 1..10: mol_type = other DNA
gcctggtcag

SEQ ID NO: 337 - DNA, 10 bp, synthetic construct
  source 1..10: mol_type = other DNA
ggcaaaaagg

SEQ ID NO: 338 - DNA, 10 bp, synthetic construct
  source 1..10: mol_type = other DNA
acccttctct

SEQ ID NO: 339 - DNA, 10 bp, synthetic construct
  source 1..10: mol_type = other DNA
tcacatagtg

SEQ ID NO: 340 - DNA, 10 bp, synthetic construct
  source 1..10: mol_type = other DNA
tcgtctgtgc

SEQ ID NO: 341 - DNA, 10 bp, synthetic construct
  source 1..10: mol_type = other DNA
tgctcggatc

SEQ ID NO: 342 - DNA, 10 bp, synthetic construct
  source 1..10: mol_type = other DNA
agcagtcccg

SEQ ID NO: 343 - DNA, 10 bp, synthetic construct
  source 1..10: mol_type = other DNA
tttgggctgt

SEQ ID NO: 344 - DNA, 10 bp, synthetic construct
  source 1..10: mol_type = other DNA
ctcacgatct

SEQ ID NO: 345 - DNA, 10 bp, synthetic construct
  source 1..10: mol_type = other DNA
tggcgcatac

SEQ ID NO: 346 - DNA, 10 bp, synthetic construct
  source 1..10: mol_type = other DNA
gcaattgaaa

SEQ ID NO: 347 - DNA, 10 bp, synthetic construct
  source 1..10: mol_type = other DNA
tcgggagacg

SEQ ID NO: 348 - DNA, 10 bp, synthetic construct
  source 1..10: mol_type = other DNA
cccggcgaaa

SEQ ID NO: 349 - DNA, 10 bp, synthetic construct
  source 1..10: mol_type = other DNA
tgatgcggaa

SEQ ID NO: 350 - DNA, 10 bp, synthetic construct
  source 1..10: mol_type = other DNA
aactgaggcg

SEQ ID NO: 351 - DNA, 10 bp, synthetic construct
  source 1..10: mol_type = other DNA
catattattt

SEQ ID NO: 352 - DNA, 10 bp, synthetic construct
  source 1..10: mol_type = other DNA
aaaagtcatt

SEQ ID NO: 353 - DNA, 10 bp, synthetic construct
  source 1..10: mol_type = other DNA
aagcggtgag

SEQ ID NO: 354 - DNA, 10 bp, synthetic construct
  source 1..10: mol_type = other DNA
aaggtaatca

SEQ ID NO: 355 - DNA, 10 bp, synthetic construct
  source 1..10: mol_type = other DNA
ctgacactta

SEQ ID NO: 356 - DNA, 10 bp, synthetic construct
  source 1..10: mol_type = other DNA
ctgttttcta

SEQ ID NO: 357 - DNA, 10 bp, synthetic construct
  source 1..10: mol_type = other DNA
cacatggcag

SEQ ID NO: 358 - DNA, 10 bp, synthetic construct
  source 1..10: mol_type = other DNA
ttcaatccgg

SEQ ID NO: 359 - DNA, 10 bp, synthetic construct
  source 1..10: mol_type = other DNA
tgtccggcat

SEQ ID NO: 360 - DNA, 10 bp, synthetic construct
  source 1..10: mol_type = other DNA
tggtaccgtg

SEQ ID NO: 361 - DNA, 10 bp, synthetic construct
  source 1..10: mol_type = other DNA
aagagatatt

SEQ ID NO: 362 - DNA, 10 bp, synthetic construct
  source 1..10: mol_type = other DNA
gatgtactac

SEQ ID NO: 363 - DNA, 10 bp, synthetic construct
  source 1..10: mol_type = other DNA
gaaatggaat

SEQ ID NO: 364 - DNA, 10 bp, synthetic construct
  source 1..10: mol_type = other DNA
ttaaaatact

SEQ ID NO: 365 - DNA, 10 bp, synthetic construct
  source 1..10: mol_type = other DNA
tgaccggaac

SEQ ID NO: 366 - DNA, 10 bp, synthetic construct
  source 1..10: mol_type = other DNA
gtcgccgcaa

SEQ ID NO: 367 - DNA, 10 bp, synthetic construct
  source 1..10: mol_type = other DNA
taggataccg

SEQ ID NO: 368 - DNA, 10 bp, synthetic construct
  source 1..10: mol_type = other DNA
agtccaattg

SEQ ID NO: 369 - DNA, 10 bp, synthetic construct
  source 1..10: mol_type = other DNA
gggggctata

SEQ ID NO: 370 - DNA, 10 bp, synthetic construct
  source 1..10: mol_type = other DNA
accttcagtt

SEQ ID NO: 371 - DNA, 10 bp, synthetic construct
  source 1..10: mol_type = other DNA
atggcaagta

SEQ ID NO: 372 - DNA, 10 bp, synthetic construct
  source 1..10: mol_type = other DNA
agaatgtttt

SEQ ID NO: 373 - DNA, 10 bp, synthetic construct
  source 1..10: mol_type = other DNA
agttcgtttg

SEQ ID NO: 374 - DNA, 10 bp, synthetic construct
  source 1..10: mol_type = other DNA
cactactgac

SEQ ID NO: 375 - DNA, 10 bp, synthetic construct
  source 1..10: mol_type = other DNA
gatcaagagc

SEQ ID NO: 376 - DNA, 10 bp, synthetic construct
  source 1..10: mol_type = other DNA
atttatcgag

SEQ ID NO: 377 - DNA, 10 bp, synthetic construct
  source 1..10: mol_type = other DNA
cctttttcca

SEQ ID NO: 378 - DNA, 10 bp, synthetic construct
  source 1..10: mol_type = other DNA
gcacagaggt

SEQ ID NO: 379 - DNA, 10 bp, synthetic construct
  source 1..10: mol_type = other DNA
tgatctgaat

SEQ ID NO: 380 - DNA, 10 bp, synthetic construct
  source 1..10: mol_type = other DNA
gttggaggga

SEQ ID NO: 381 - DNA, 10 bp, synthetic construct
  source 1..10: mol_type = other DNA
ttttgaaggt

SEQ ID NO: 382 - DNA, 10 bp, synthetic construct
  source 1..10: mol_type = other DNA
taagtcctaa

SEQ ID NO: 383 - DNA, 10 bp, synthetic construct
  source 1..10: mol_type = other DNA
ggtgttaggg

SEQ ID NO: 384 - DNA, 10 bp, synthetic construct
  source 1..10: mol_type = other DNA
tgtatgcacc

SEQ ID NO: 385 - DNA, 10 bp, synthetic construct
  source 1..10: mol_type = other DNA
ccgtgccatt

SEQ ID NO: 386 - DNA, 10 bp, synthetic construct
  source 1..10: mol_type = other DNA
gaaatcaccc

SEQ ID NO: 387 - DNA, 10 bp, synthetic construct
  source 1..10: mol_type = other DNA
tttgcacgtg

SEQ ID NO: 388 - DNA, 10 bp, synthetic construct
  source 1..10: mol_type = other DNA
cgtctgtttt

SEQ ID NO: 389 - DNA, 10 bp, synthetic construct
  source 1..10: mol_type = other DNA
ctacaccaca

SEQ ID NO: 390 - DNA, 10 bp, synthetic construct
  source 1..10: mol_type = other DNA
tgctacaggg

SEQ ID NO: 391 - DNA, 10 bp, synthetic construct
  source 1..10: mol_type = other DNA
gggaatatat

SEQ ID NO: 392 - DNA, 10 bp, synthetic construct
  source 1..10: mol_type = other DNA
tcatgtattt

SEQ ID NO: 393 - DNA, 10 bp, synthetic construct
  source 1..10: mol_type = other DNA
tctccgttta

SEQ ID NO: 394 - DNA, 10 bp, synthetic construct
  source 1..10: mol_type = other DNA
tacctctcgc

SEQ ID NO: 395 - DNA, 10 bp, synthetic construct
  source 1..10: mol_type = other DNA
gcttcaaccg

SEQ ID NO: 396 - DNA, 10 bp, synthetic construct
  source 1..10: mol_type = other DNA
atgaagctac

SEQ ID NO: 397 - DNA, 10 bp, synthetic construct
  source 1..10: mol_type = other DNA
cggtacaact

SEQ ID NO: 398 - DNA, 10 bp, synthetic construct
  source 1..10: mol_type = other DNA
gtgtggtcgt

SEQ ID NO: 399 - DNA, 10 bp, synthetic construct
  source 1..10: mol_type = other DNA
ggggtcatgt

SEQ ID NO: 400 - DNA, 10 bp, synthetic construct
  source 1..10: mol_type = other DNA
aggcagccca

SEQ ID NO: 401 - DNA, 10 bp, synthetic construct
  source 1..10: mol_type = other DNA
caagcacgat

SEQ ID NO: 402 - DNA, 10 bp, synthetic construct
  source 1..10: mol_type = other DNA
tcaaatggat

SEQ ID NO: 403 - DNA, 10 bp, synthetic construct
  source 1..10: mol_type = other DNA
ggactgaata

SEQ ID NO: 404 - DNA, 10 bp, synthetic construct
  source 1..10: mol_type = other DNA
ccgtagacgt

SEQ ID NO: 405 - DNA, 10 bp, synthetic construct
  source 1..10: mol_type = other DNA
cggcgtaccg

SEQ ID NO: 406 - DNA, 10 bp, synthetic construct
  source 1..10: mol_type = other DNA
ggcggcgccc

SEQ ID NO: 407 - DNA, 10 bp, synthetic construct
  source 1..10: mol_type = other DNA
agacttgatc

SEQ ID NO: 408 - DNA, 10 bp, synthetic construct
  source 1..10: mol_type = other DNA
accttgcaca

SEQ ID NO: 409 - DNA, 10 bp, synthetic construct
  source 1..10: mol_type = other DNA
taaggtgagt

SEQ ID NO: 410 - DNA, 10 bp, synthetic construct
  source 1..10: mol_type = other DNA
ttgttgtttc

SEQ ID NO: 411 - DNA, 10 bp, synthetic construct
  source 1..10: mol_type = other DNA
gagggaatac

SEQ ID NO: 412 - DNA, 10 bp, synthetic construct
  source 1..10: mol_type = other DNA
ctcgtacgcg

SEQ ID NO: 413 - DNA, 10 bp, synthetic construct
  source 1..10: mol_type = other DNA
ccgcggttta

SEQ ID NO: 414 - DNA, 10 bp, synthetic construct
  source 1..10: mol_type = other DNA
ttaaagttaa

SEQ ID NO: 415 - DNA, 10 bp, synthetic construct
  source 1..10: mol_type = other DNA
gcatatgggt

SEQ ID NO: 416 - DNA, 10 bp, synthetic construct
  source 1..10: mol_type = other DNA
agtctgagcc

SEQ ID NO: 417 - DNA, 10 bp, synthetic construct
  source 1..10: mol_type = other DNA
tgtcggttcg

SEQ ID NO: 418 - DNA, 10 bp, synthetic construct
  source 1..10: mol_type = other DNA
ggtctcaacc

SEQ ID NO: 419 - DNA, 10 bp, synthetic construct
  source 1..10: mol_type = other DNA
gtaacggcat

SEQ ID NO: 420 - DNA, 10 bp, synthetic construct
  source 1..10: mol_type = other DNA
acactgagaa

SEQ ID NO: 421 - DNA, 10 bp, synthetic construct
  source 1..10: mol_type = other DNA
cccaacgtcg

SEQ ID NO: 422 - DNA, 10 bp, synthetic construct
  source 1..10: mol_type = other DNA
aagaaactgc

SEQ ID NO: 423 - DNA, 10 bp, synthetic construct
  source 1..10: mol_type = other DNA
accagcccac

SEQ ID NO: 424 - DNA, 10 bp, synthetic construct
  source 1..10: mol_type = other DNA
tgtagttact

SEQ ID NO: 425 - DNA, 10 bp, synthetic construct
  source 1..10: mol_type = other DNA
ggctagaggc

SEQ ID NO: 426 - DNA, 10 bp, synthetic construct
  source 1..10: mol_type = other DNA
gttcggcaga

SEQ ID NO: 427 - DNA, 10 bp, synthetic construct
  source 1..10: mol_type = other DNA
ccaaaataga

SEQ ID NO: 428 - DNA, 10 bp, synthetic construct
  source 1..10: mol_type = other DNA
cccatataac

SEQ ID NO: 429 - DNA, 10 bp, synthetic construct
  source 1..10: mol_type = other DNA
gtcactaccg

SEQ ID NO: 430 - DNA, 10 bp, synthetic construct
  source 1..10: mol_type = other DNA
gtagtgtggc

SEQ ID NO: 431 - DNA, 10 bp, synthetic construct
  source 1..10: mol_type = other DNA
caatctcata

SEQ ID NO: 432 - DNA, 10 bp, synthetic construct
  source 1..10: mol_type = other DNA
ccatgttata

SEQ ID NO: 433 - DNA, 10 bp, synthetic construct
  source 1..10: mol_type = other DNA
taagcagtgg

SEQ ID NO: 434 - DNA, 10 bp, synthetic construct
  source 1..10: mol_type = other DNA
tcggcggcta

SEQ ID NO: 435 - DNA, 10 bp, synthetic construct
  source 1..10: mol_type = other DNA
tattaaatgc

SEQ ID NO: 436 - DNA, 10 bp, synthetic construct
  source 1..10: mol_type = other DNA
gtcgccatta

SEQ ID NO: 437 - DNA, 10 bp, synthetic construct
  source 1..10: mol_type = other DNA
ggcgtcgttc

SEQ ID NO: 438 - DNA, 10 bp, synthetic construct
  source 1..10: mol_type = other DNA
ctagtagata

SEQ ID NO: 439 - DNA, 10 bp, synthetic construct
  source 1..10: mol_type = other DNA
tcgtcagtat

SEQ ID NO: 440 - DNA, 10 bp, synthetic construct
  source 1..10: mol_type = other DNA
ggggtatcgg

SEQ ID NO: 441 - DNA, 10 bp, synthetic construct
  source 1..10: mol_type = other DNA
tgctctgcca

SEQ ID NO: 442 - DNA, 10 bp, synthetic construct
  source 1..10: mol_type = other DNA
tgccgtaact

SEQ ID NO: 443 - DNA, 10 bp, synthetic construct
  source 1..10: mol_type = other DNA
cggtacaggc

SEQ ID NO: 444 - DNA, 10 bp, synthetic construct
  source 1..10: mol_type = other DNA
tcctaatttg

SEQ ID NO: 445 - DNA, 10 bp, synthetic construct
  source 1..10: mol_type = other DNA
tctttctgga

SEQ ID NO: 446 - DNA, 10 bp, synthetic construct
  source 1..10: mol_type = other DNA
ccgcgacttg

SEQ ID NO: 447 - DNA, 10 bp, synthetic construct
  source 1..10: mol_type = other DNA
acctatagcg

SEQ ID NO: 448 - DNA, 10 bp, synthetic construct
  source 1..10: mol_type = other DNA
gccggcacct

SEQ ID NO: 449 - DNA, 10 bp, synthetic construct
  source 1..10: mol_type = other DNA
tttgataggc

SEQ ID NO: 450 - DNA, 10 bp, synthetic construct
  source 1..10: mol_type = other DNA
actgtgagct

SEQ ID NO: 451 - DNA, 10 bp, synthetic construct
  source 1..10: mol_type = other DNA
ttatcgttca

SEQ ID NO: 452 - DNA, 10 bp, synthetic construct
  source 1..10: mol_type = other DNA
actagtggcc

SEQ ID NO: 453 - DNA, 10 bp, synthetic construct
  source 1..10: mol_type = other DNA
cctccgtggt

SEQ ID NO: 454 - DNA, 10 bp, synthetic construct
  source 1..10: mol_type = other DNA
ttagggtatg

SEQ ID NO: 455 - DNA, 10 bp, synthetic construct
  source 1..10: mol_type = other DNA
gaatcaggcg

SEQ ID NO: 456 - DNA, 10 bp, synthetic construct
  source 1..10: mol_type = other DNA
ggctgaccaa

SEQ ID NO: 457 - DNA, 10 bp, synthetic construct
  source 1..10: mol_type = other DNA
tgccagaccg

SEQ ID NO: 458 - DNA, 10 bp, synthetic construct
  source 1..10: mol_type = other DNA
tccctacgcg

SEQ ID NO: 459 - DNA, 10 bp, synthetic construct
  source 1..10: mol_type = other DNA
tccgctggag

SEQ ID NO: 460 - DNA, 10 bp, synthetic construct
  source 1..10: mol_type = other DNA
ggatcaaaac

SEQ ID NO: 461 - DNA, 10 bp, synthetic construct
  source 1..10: mol_type = other DNA
ttcacctcac

SEQ ID NO: 462 - DNA, 10 bp, synthetic construct
  source 1..10: mol_type = other DNA
gacacacggc

SEQ ID NO: 463 - DNA, 10 bp, synthetic construct
  source 1..10: mol_type = other DNA
tgggcgatta

SEQ ID NO: 464 - DNA, 10 bp, synthetic construct
  source 1..10: mol_type = other DNA
taagatcttc

SEQ ID NO: 465 - DNA, 10 bp, synthetic construct
  source 1..10: mol_type = other DNA
ctccgactac

SEQ ID NO: 466 - DNA, 10 bp, synthetic construct
  source 1..10: mol_type = other DNA
gggccatcat

SEQ ID NO: 467 - DNA, 10 bp, synthetic construct
  source 1..10: mol_type = other DNA
tcaggccaga

SEQ ID NO: 468 - DNA, 10 bp, synthetic construct
  source 1..10: mol_type = other DNA
cttgtggggc

SEQ ID NO: 469 - DNA, 10 bp, synthetic construct
  source 1..10: mol_type = other DNA
agatagtctg

SEQ ID NO: 470 - DNA, 10 bp, synthetic construct
  source 1..10: mol_type = other DNA
gcgtcaaagt

SEQ ID NO: 471 - DNA, 10 bp, synthetic construct
  source 1..10: mol_type = other DNA
acgaaaattt

SEQ ID NO: 472 - DNA, 10 bp, synthetic construct
  source 1..10: mol_type = other DNA
gagtctggtg

SEQ ID NO: 473 - DNA, 10 bp, synthetic construct
  source 1..10: mol_type = other DNA
atcgagcgac

SEQ ID NO: 474 - DNA, 10 bp, synthetic construct
  source 1..10: mol_type = other DNA
ggtcctcaga

SEQ ID NO: 475 - DNA, 10 bp, synthetic construct
  source 1..10: mol_type = other DNA
tgattttgtc

SEQ ID NO: 476 - DNA, 10 bp, synthetic construct
  source 1..10: mol_type = other DNA
gcatttctca

SEQ ID NO: 477 - DNA, 10 bp, synthetic construct
  source 1..10: mol_type = other DNA
gcatgccagt

SEQ ID NO: 478 - DNA, 10 bp, synthetic construct
  source 1..10: mol_type = other DNA
attagacgac

SEQ ID NO: 479 - DNA, 10 bp, synthetic construct
  source 1..10: mol_type = other DNA
aaagcccata

SEQ ID NO: 480 - DNA, 10 bp, synthetic construct
  source 1..10: mol_type = other DNA
cactacattc

SEQ ID NO: 481 - DNA, 10 bp, synthetic construct
  source 1..10: mol_type = other DNA
cacggtttct

SEQ ID NO: 482 - DNA, 10 bp, synthetic construct
  source 1..10: mol_type = other DNA
cccaccagtg

SEQ ID NO: 483 - DNA, 10 bp, synthetic construct
  source 1..10: mol_type = other DNA
ctcacttgtc

SEQ ID NO: 484 - DNA, 10 bp, synthetic construct
  source 1..10: mol_type = other DNA
gatagactct

SEQ ID NO: 485 - DNA, 10 bp, synthetic construct
  source 1..10: mol_type = other DNA
atttccattt

SEQ ID NO: 486 - DNA, 10 bp, synthetic construct
  source 1..10: mol_type = other DNA
atatgtggcc

SEQ ID NO: 487 - DNA, 10 bp, synthetic construct
  source 1..10: mol_type = other DNA
cgggacgaac

SEQ ID NO: 488 - DNA, 10 bp, synthetic construct
  source 1..10: mol_type = other DNA
agaaccgtga

SEQ ID NO: 489 - DNA, 10 bp, synthetic construct
  source 1..10: mol_type = other DNA
tagtgtactg

SEQ ID NO: 490 - DNA, 10 bp, synthetic construct
  source 1..10: mol_type = other DNA
aactaatcga

SEQ ID NO: 491 - DNA, 10 bp, synthetic construct
  source 1..10: mol_type = other DNA
cgaagtgacg

SEQ ID NO: 492 - DNA, 10 bp, synthetic construct
  source 1..10: mol_type = other DNA
cggagcctcg

SEQ ID NO: 493 - DNA, 10 bp, synthetic construct
  source 1..10: mol_type = other DNA
atcacacgag

SEQ ID NO: 494 - DNA, 10 bp, synthetic construct
  source 1..10: mol_type = other DNA
cgacgagttc

SEQ ID NO: 495 - DNA, 10 bp, synthetic construct
  source 1..10: mol_type = other DNA
gcttcccgtg

SEQ ID NO: 496 - DNA, 10 bp, synthetic construct
  source 1..10: mol_type = other DNA
gattcatacc

SEQ ID NO: 497 - DNA, 10 bp, synthetic construct
  source 1..10: mol_type = other DNA
gagagaagcg

SEQ ID NO: 498 - DNA, 10 bp, synthetic construct
  source 1..10: mol_type = other DNA
gaagtggcct

SEQ ID NO: 499 - DNA, 10 bp, synthetic construct
  source 1..10: mol_type = other DNA
ggacgacgcc

SEQ ID NO: 500 - DNA, 10 bp, synthetic construct
  source 1..10: mol_type = other DNA
tagggtctca

SEQ ID NO: 501 - DNA, 10 bp, synthetic construct
  source 1..10: mol_type = other DNA
aactacaggt

SEQ ID NO: 502 - DNA, 10 bp, synthetic construct
  source 1..10: mol_type = other DNA
gtggcctgtg

SEQ ID NO: 503 - DNA, 10 bp, synthetic construct
  source 1..10: mol_type = other DNA
ctttaccagc

SEQ ID NO: 504 - DNA, 10 bp, synthetic construct
  source 1..10: mol_type = other DNA
cgcgttactg

SEQ ID NO: 505 - DNA, 10 bp, synthetic construct
  source 1..10: mol_type = other DNA
ttgctcccgt

SEQ ID NO: 506 - DNA, 10 bp, synthetic construct
  source 1..10: mol_type = other DNA
catcaaacaa

SEQ ID NO: 507 - DNA, 10 bp, synthetic construct
  source 1..10: mol_type = other DNA
gctttatgat

SEQ ID NO: 508 - DNA, 10 bp, synthetic construct
  source 1..10: mol_type = other DNA
ctgcatactg

SEQ ID NO: 509 - DNA, 10 bp, synthetic construct
  source 1..10: mol_type = other DNA
ggtggctcag

SEQ ID NO: 510 - DNA, 10 bp, synthetic construct
  source 1..10: mol_type = other DNA
ggacgatcaa

SEQ ID NO: 511 - DNA, 10 bp, synthetic construct
  source 1..10: mol_type = other DNA
ccgactggtg

SEQ ID NO: 512 - DNA, 10 bp, synthetic construct
  source 1..10: mol_type = other DNA
ggaacaaccg

SEQ ID NO: 513 - DNA, 10 bp, synthetic construct
  source 1..10: mol_type = other DNA
gaacgagacc

SEQ ID NO: 514 - DNA, 10 bp, synthetic construct
  source 1..10: mol_type = other DNA
caccaagaaa

SEQ ID NO: 515 - DNA, 10 bp, synthetic construct
  source 1..10: mol_type = other DNA
atgcattacc

SEQ ID NO: 516 - DNA, 10 bp, synthetic construct
  source 1..10: mol_type = other DNA
gtatcatgcc

SEQ ID NO: 517 - DNA, 10 bp, synthetic construct
  source 1..10: mol_type = other DNA
agtagatgtt

SEQ ID NO: 518 - DNA, 10 bp, synthetic construct
  source 1..10: mol_type = other DNA
ctctagatgt

SEQ ID NO: 519 - DNA, 10 bp, synthetic construct
  source 1..10: mol_type = other DNA
gctacttgtg

SEQ ID NO: 520 - DNA, 10 bp, synthetic construct
  source 1..10: mol_type = other DNA
tatgaaacgt

SEQ ID NO: 521 - DNA, 10 bp, synthetic construct
  source 1..10: mol_type = other DNA
cctcgttgat

SEQ ID NO: 522 - DNA, 10 bp, synthetic construct
  source 1..10: mol_type = other DNA
ctagagccat

SEQ ID NO: 523 - DNA, 10 bp, synthetic construct
  source 1..10: mol_type = other DNA
tagagttata

SEQ ID NO: 524 - DNA, 10 bp, synthetic construct
  source 1..10: mol_type = other DNA
aacgagaggc

SEQ ID NO: 525 - DNA, 10 bp, synthetic construct
  source 1..10: mol_type = other DNA
ggtctaccgt

SEQ ID NO: 526 - DNA, 10 bp, synthetic construct
  source 1..10: mol_type = other DNA
gccccctcac

SEQ ID NO: 527 - DNA, 10 bp, synthetic construct
  source 1..10: mol_type = other DNA
cataggaatt

SEQ ID NO: 528 - DNA, 10 bp, synthetic construct
  source 1..10: mol_type = other DNA
tccggctcgt

SEQ ID NO: 529 - DNA, 10 bp, synthetic construct
  source 1..10: mol_type = other DNA
tgagagtcgg

SEQ ID NO: 530 - DNA, 10 bp, synthetic construct
  source 1..10: mol_type = other DNA
cgtagaaata

SEQ ID NO: 531 - DNA, 10 bp, synthetic construct
  source 1..10: mol_type = other DNA
ctttacatga

SEQ ID NO: 532 - DNA, 10 bp, synthetic construct
  source 1..10: mol_type = other DNA
gagcgccgtc

SEQ ID NO: 533 - DNA, 10 bp, synthetic construct
  source 1..10: mol_type = other DNA
ggctctcggc

SEQ ID NO: 534 - DNA, 10 bp, synthetic construct
  source 1..10: mol_type = other DNA
agagcttgtt

SEQ ID NO: 535 - DNA, 10 bp, synthetic construct
  source 1..10: mol_type = other DNA
aatcagccac

SEQ ID NO: 536 - DNA, 10 bp, synthetic construct
  source 1..10: mol_type = other DNA
agaagagcca

SEQ ID NO: 537 - DNA, 10 bp, synthetic construct
  source 1..10: mol_type = other DNA
tcgtatgagt

SEQ ID NO: 538 - DNA, 10 bp, synthetic construct
  source 1..10: mol_type = other DNA
ttcttcctcg

SEQ ID NO: 539 - DNA, 10 bp, synthetic construct
  source 1..10: mol_type = other DNA
acacaaaagc

SEQ ID NO: 540 - DNA, 10 bp, synthetic construct
  source 1..10: mol_type = other DNA
cgcgggaccc

SEQ ID NO: 541 - DNA, 10 bp, synthetic construct
  source 1..10: mol_type = other DNA
gtcgcgacac

SEQ ID NO: 542 - DNA, 10 bp, synthetic construct
  source 1..10: mol_type = other DNA
ccggaggaaa

SEQ ID NO: 543 - DNA, 10 bp, synthetic construct
  source 1..10: mol_type = other DNA
cggcgtatga

SEQ ID NO: 544 - DNA, 10 bp, synthetic construct
  source 1..10: mol_type = other DNA
taggcattct

SEQ ID NO: 545 - DNA, 10 bp, synthetic construct
  source 1..10: mol_type = other DNA
aaaggaggga

SEQ ID NO: 546 - DNA, 10 bp, synthetic construct
  source 1..10: mol_type = other DNA
acctttacgg

SEQ ID NO: 547 - DNA, 10 bp, synthetic construct
  source 1..10: mol_type = other DNA
ctaccgttaa

SEQ ID NO: 548 - DNA, 10 bp, synthetic construct
  source 1..10: mol_type = other DNA
gagcttcgcc

SEQ ID NO: 549 - DNA, 10 bp, synthetic construct
  source 1..10: mol_type = other DNA
gccatagaag

SEQ ID NO: 550 - DNA, 10 bp, synthetic construct
  source 1..10: mol_type = other DNA
tttagcgtat

SEQ ID NO: 551 - DNA, 10 bp, synthetic construct
  source 1..10: mol_type = other DNA
gcaaacagat

SEQ ID NO: 552 - DNA, 10 bp, synthetic construct
  source 1..10: mol_type = other DNA
taggtcatgg

SEQ ID NO: 553 - DNA, 10 bp, synthetic construct
  source 1..10: mol_type = other DNA
ctctaacaga

SEQ ID NO: 554 - DNA, 10 bp, synthetic construct
  source 1..10: mol_type = other DNA
ggctcatgaa

SEQ ID NO: 555 - DNA, 10 bp, synthetic construct
  source 1..10: mol_type = other DNA
caatgtctca

SEQ ID NO: 556 - DNA, 10 bp, synthetic construct
  source 1..10: mol_type = other DNA
tgatcgtatt

SEQ ID NO: 557 - DNA, 10 bp, synthetic construct
  source 1..10: mol_type = other DNA
gcgcttttca

SEQ ID NO: 558 - DNA, 10 bp, synthetic construct
  source 1..10: mol_type = other DNA
aagattatat

SEQ ID NO: 559 - DNA, 10 bp, synthetic construct
  source 1..10: mol_type = other DNA
actagctgac

SEQ ID NO: 560 - DNA, 10 bp, synthetic construct
  source 1..10: mol_type = other DNA
ggtgagctca

SEQ ID NO: 561 - DNA, 10 bp, synthetic construct
  source 1..10: mol_type = other DNA
cgctttcgct

SEQ ID NO: 562 - DNA, 10 bp, synthetic construct
  source 1..10: mol_type = other DNA
tgattcaaaa

SEQ ID NO: 563 - DNA, 10 bp, synthetic construct
  source 1..10: mol_type = other DNA
actgaacagg

SEQ ID NO: 564 - DNA, 10 bp, synthetic construct
  source 1..10: mol_type = other DNA
attcgagcta

SEQ ID NO: 565 - DNA, 10 bp, synthetic construct
  source 1..10: mol_type = other DNA
tgtaggctaa

SEQ ID NO: 566 - DNA, 10 bp, synthetic construct
  source 1..10: mol_type = other DNA
acaaagcttt

SEQ ID NO: 567 - DNA, 10 bp, synthetic construct
  source 1..10: mol_type = other DNA
gcccgaggga

SEQ ID NO: 568 - DNA, 10 bp, synthetic construct
  source 1..10: mol_type = other DNA
gcccgctggg

SEQ ID NO: 569 - DNA, 10 bp, synthetic construct
  source 1..10: mol_type = other DNA
accccgctga

SEQ ID NO: 570 - DNA, 10 bp, synthetic construct
  source 1..10: mol_type = other DNA
cttatgccct

SEQ ID NO: 571 - DNA, 10 bp, synthetic construct
  source 1..10: mol_type = other DNA
ccgccatagc

SEQ ID NO: 572 - DNA, 10 bp, synthetic construct
  source 1..10: mol_type = other DNA
cttaatgatt

SEQ ID NO: 573 - DNA, 10 bp, synthetic construct
  source 1..10: mol_type = other DNA
cagtccacaa

SEQ ID NO: 574 - DNA, 10 bp, synthetic construct
  source 1..10: mol_type = other DNA
atggacggac

SEQ ID NO: 575 - DNA, 10 bp, synthetic construct
  source 1..10: mol_type = other DNA
cggcctctcg

SEQ ID NO: 576 - DNA, 10 bp, synthetic construct
  source 1..10: mol_type = other DNA
tagtcgccat

SEQ ID NO: 577 - DNA, 10 bp, synthetic construct
  source 1..10: mol_type = other DNA
gttgatcttc

SEQ ID NO: 578 - DNA, 10 bp, synthetic construct
  source 1..10: mol_type = other DNA
acttgccaag

SEQ ID NO: 579 - DNA, 10 bp, synthetic construct
  source 1..10: mol_type = other DNA
atgactggtt

SEQ ID NO: 580 - DNA, 10 bp, synthetic construct
  source 1..10: mol_type = other DNA
tgtcgtagga

SEQ ID NO: 581 - DNA, 10 bp, synthetic construct
  source 1..10: mol_type = other DNA
agcaaacacg

SEQ ID NO: 582 - DNA, 10 bp, synthetic construct
  source 1..10: mol_type = other DNA
tactgatgaa

SEQ ID NO: 583 - DNA, 10 bp, synthetic construct
  source 1..10: mol_type = other DNA
gtatcccata

SEQ ID NO: 584 - DNA, 10 bp, synthetic construct
  source 1..10: mol_type = other DNA
tagccaggtt

SEQ ID NO: 585 - DNA, 10 bp, synthetic construct
  source 1..10: mol_type = other DNA
cgtgtggcga

SEQ ID NO: 586 - DNA, 10 bp, synthetic construct
  source 1..10: mol_type = other DNA
atcgaattgc

SEQ ID NO: 587 - DNA, 10 bp, synthetic construct
  source 1..10: mol_type = other DNA
ccccaatatt

SEQ ID NO: 588 - DNA, 10 bp, synthetic construct
  source 1..10: mol_type = other DNA
cccgtttctc

SEQ ID NO: 589 - DNA, 10 bp, synthetic construct
  source 1..10: mol_type = other DNA
tccgcatcta

SEQ ID NO: 590 - DNA, 10 bp, synthetic construct
  source 1..10: mol_type = other DNA
caagcctcat

SEQ ID NO: 591 - DNA, 10 bp, synthetic construct
  source 1..10: mol_type = other DNA
tttcaatccc

SEQ ID NO: 592 - DNA, 10 bp, synthetic construct
  source 1..10: mol_type = other DNA
ccttcccatc

SEQ ID NO: 593 - DNA, 10 bp, synthetic construct
  source 1..10: mol_type = other DNA
aggtacaaga

SEQ ID NO: 594 - DNA, 10 bp, synthetic construct
  source 1..10: mol_type = other DNA
gtgtaatgga

SEQ ID NO: 595 - DNA, 10 bp, synthetic construct
  source 1..10: mol_type = other DNA
aaactgagct

SEQ ID NO: 596 - DNA, 10 bp, synthetic construct
  source 1..10: mol_type = other DNA
atctctgccc

SEQ ID NO: 597 - DNA, 10 bp, synthetic construct
  source 1..10: mol_type = other DNA
cgacatttgc

SEQ ID NO: 598 - DNA, 10 bp, synthetic construct
  source 1..10: mol_type = other DNA
tgtgaacccg

SEQ ID NO: 599 - DNA, 10 bp, synthetic construct
  source 1..10: mol_type = other DNA
tgacacccca

SEQ ID NO: 600 - DNA, 10 bp, synthetic construct
  source 1..10: mol_type = other DNA
taggccaaag

SEQ ID NO: 601 - DNA, 10 bp, synthetic construct
  source 1..10: mol_type = other DNA
gaaattgtag

SEQ ID NO: 602 - DNA, 10 bp, synthetic construct
  source 1..10: mol_type = other DNA
gcgtctgatt

SEQ ID NO: 603 - DNA, 10 bp, synthetic construct
  source 1..10: mol_type = other DNA
tctcattgtt

SEQ ID NO: 604 - DNA, 10 bp, synthetic construct
  source 1..10: mol_type = other DNA
ctgacatctc

SEQ ID NO: 605 - DNA, 10 bp, synthetic construct
  source 1..10: mol_type = other DNA
gtatccagtg

SEQ ID NO: 606 - DNA, 10 bp, synthetic construct
  source 1..10: mol_type = other DNA
gatggccgtt

SEQ ID NO: 607 - DNA, 10 bp, synthetic construct
  source 1..10: mol_type = other DNA
tcaccctctc

SEQ ID NO: 608 - DNA, 10 bp, synthetic construct
  source 1..10: mol_type = other DNA
ggcactattc

SEQ ID NO: 609 - DNA, 10 bp, synthetic construct
  source 1..10: mol_type = other DNA
aaataactgt

SEQ ID NO: 610 - DNA, 10 bp, synthetic construct
  source 1..10: mol_type = other DNA
cagctccatt

SEQ ID NO: 611 - DNA, 10 bp, synthetic construct
  source 1..10: mol_type = other DNA
ctcttgactc

SEQ ID NO: 612 - DNA, 10 bp, synthetic construct
  source 1..10: mol_type = other DNA
tttcctatac

SEQ ID NO: 613 - DNA, 10 bp, synthetic construct
  source 1..10: mol_type = other DNA
ccatacccga

SEQ ID NO: 614 - DNA, 10 bp, synthetic construct
  source 1..10: mol_type = other DNA
tcgccgagcg

SEQ ID NO: 615 - DNA, 10 bp, synthetic construct
  source 1..10: mol_type = other DNA
cgctgaagcc

SEQ ID NO: 616 - DNA, 10 bp, synthetic construct
  source 1..10: mol_type = other DNA
tctggcccca

SEQ ID NO: 617 - DNA, 10 bp, synthetic construct
  source 1..10: mol_type = other DNA
gctacattga

SEQ ID NO: 618 - DNA, 10 bp, synthetic construct
  source 1..10: mol_type = other DNA
cgcatcataa

SEQ ID NO: 619 - DNA, 10 bp, synthetic construct
  source 1..10: mol_type = other DNA
gcaaagggcc

SEQ ID NO: 620 - DNA, 10 bp, synthetic construct
  source 1..10: mol_type = other DNA
aacggcgcag

SEQ ID NO: 621 - DNA, 10 bp, synthetic construct
  source 1..10: mol_type = other DNA
cgactgacat

SEQ ID NO: 622 - DNA, 10 bp, synthetic construct
  source 1..10: mol_type = other DNA
atgacagggc

SEQ ID NO: 623 - DNA, 10 bp, synthetic construct
  source 1..10: mol_type = other DNA
caagttctcc

SEQ ID NO: 624 - DNA, 10 bp, synthetic construct
  source 1..10: mol_type = other DNA
tcgccgcttt

SEQ ID NO: 625 - DNA, 10 bp, synthetic construct
  source 1..10: mol_type = other DNA
atgccggaaa

SEQ ID NO: 626 - DNA, 10 bp, synthetic construct
  source 1..10: mol_type = other DNA
gcggttacta

SEQ ID NO: 627 - DNA, 10 bp, synthetic construct
  source 1..10: mol_type = other DNA
gacattacaa

SEQ ID NO: 628 - DNA, 10 bp, synthetic construct
  source 1..10: mol_type = other DNA
cagagagggc

SEQ ID NO: 629 - DNA, 10 bp, synthetic construct
  source 1..10: mol_type = other DNA
gcaccgcctc

SEQ ID NO: 630 - DNA, 10 bp, synthetic construct
  source 1..10: mol_type = other DNA
cggtccgagc

SEQ ID NO: 631 - DNA, 10 bp, synthetic construct
  source 1..10: mol_type = other DNA
tgtccggtgc

SEQ ID NO: 632 - DNA, 10 bp, synthetic construct
  source 1..10: mol_type = other DNA
ggtcggttgc

SEQ ID NO: 633 - DNA, 10 bp, synthetic construct
  source 1..10: mol_type = other DNA
gctcagctaa

SEQ ID NO: 634 - DNA, 10 bp, synthetic construct
  source 1..10: mol_type = other DNA
agcagttcgt

SEQ ID NO: 635 - DNA, 10 bp, synthetic construct
  source 1..10: mol_type = other DNA
aaatcgatga

SEQ ID NO: 636 - DNA, 10 bp, synthetic construct
  source 1..10: mol_type = other DNA
gctcggtatg

SEQ ID NO: 637 - DNA, 10 bp, synthetic construct
  source 1..10: mol_type = other DNA
cccgccgcgg

SEQ ID NO: 638 - DNA, 10 bp, synthetic construct
  source 1..10: mol_type = other DNA
gtgtgatagg

SEQ ID NO: 639 - DNA, 10 bp, synthetic construct
  source 1..10: mol_type = other DNA
ttggactcca

SEQ ID NO: 640 - DNA, 10 bp, synthetic construct
  source 1..10: mol_type = other DNA
tgcttatcta

SEQ ID NO: 641 - DNA, 10 bp, synthetic construct
  source 1..10: mol_type = other DNA
caaaaggcgt

SEQ ID NO: 642 - DNA, 10 bp, synthetic construct
  source 1..10: mol_type = other DNA
tagggggcct

SEQ ID NO: 643 - DNA, 10 bp, synthetic construct
  source 1..10: mol_type = other DNA
aagtattaat

SEQ ID NO: 644 - DNA, 10 bp, synthetic construct
  source 1..10: mol_type = other DNA
gtttagcccg

SEQ ID NO: 645 - DNA, 10 bp, synthetic construct
  source 1..10: mol_type = other DNA
cgctaatatg

SEQ ID NO: 646 - DNA, 10 bp, synthetic construct
  source 1..10: mol_type = other DNA
acaacacgtt

SEQ ID NO: 647 - DNA, 10 bp, synthetic construct
  source 1..10: mol_type = other DNA
agagatgctc

SEQ ID NO: 648 - DNA, 10 bp, synthetic construct
  source 1..10: mol_type = other DNA
tgcctgatat

SEQ ID NO: 649 - DNA, 10 bp, synthetic construct
  source 1..10: mol_type = other DNA
cttgtaagta

SEQ ID NO: 650 - DNA, 10 bp, synthetic construct
  source 1..10: mol_type = other DNA
catattgccg

SEQ ID NO: 651 - DNA, 10 bp, synthetic construct
  source 1..10: mol_type = other DNA
cttagaaagt

SEQ ID NO: 652 - DNA, 10 bp, synthetic construct
  source 1..10: mol_type = other DNA
atgttgtatt

SEQ ID NO: 653 - DNA, 10 bp, synthetic construct
  source 1..10: mol_type = other DNA
cgcattgaag

SEQ ID NO: 654 - DNA, 10 bp, synthetic construct
  source 1..10: mol_type = other DNA
ttatgttggt

SEQ ID NO: 655 - DNA, 10 bp, synthetic construct
  source 1..10: mol_type = other DNA
tcgcctcaga

SEQ ID NO: 656 - DNA, 10 bp, synthetic construct
  source 1..10: mol_type = other DNA
ttcgttgagg

SEQ ID NO: 657 - DNA, 10 bp, synthetic construct
  source 1..10: mol_type = other DNA
ggtgccgggc

SEQ ID NO: 658 - DNA, 10 bp, synthetic construct
  source 1..10: mol_type = other DNA
accattgtaa

SEQ ID NO: 659 - DNA, 10 bp, synthetic construct
  source 1..10: mol_type = other DNA
ttgattgtca

SEQ ID NO: 660 - DNA, 10 bp, synthetic construct
  source 1..10: mol_type = other DNA
cggctcacct

SEQ ID NO: 661 - DNA, 10 bp, synthetic construct
  source 1..10: mol_type = other DNA
ctatcacatg

SEQ ID NO: 662 - DNA, 10 bp, synthetic construct
  source 1..10: mol_type = other DNA
gtagacagaa

SEQ ID NO: 663 - DNA, 10 bp, synthetic construct
  source 1..10: mol_type = other DNA
cctttaccaa

SEQ ID NO: 664 - DNA, 10 bp, synthetic construct
  source 1..10: mol_type = other DNA
gcacatcgac

SEQ ID NO: 665 - DNA, 10 bp, synthetic construct
  source 1..10: mol_type = other DNA
tctcactttc

SEQ ID NO: 666 - DNA, 10 bp, synthetic construct
  source 1..10: mol_type = other DNA
ttcgagtact

SEQ ID NO: 667 - DNA, 10 bp, synthetic construct
  source 1..10: mol_type = other DNA
tagaagagca

SEQ ID NO: 668 - DNA, 10 bp, synthetic construct
  source 1..10: mol_type = other DNA
aaccccacca

SEQ ID NO: 669 - DNA, 10 bp, synthetic construct
  source 1..10: mol_type = other DNA
ctgtatcagt

SEQ ID NO: 670 - DNA, 10 bp, synthetic construct
  source 1..10: mol_type = other DNA
acataatgag

SEQ ID NO: 671 - DNA, 10 bp, synthetic construct
  source 1..10: mol_type = other DNA
agccttccgc

SEQ ID NO: 672 - DNA, 10 bp, synthetic construct
  source 1..10: mol_type = other DNA
cagtgctttt

SEQ ID NO: 673 - DNA, 10 bp, synthetic construct
  source 1..10: mol_type = other DNA
tagtccgtgt

SEQ ID NO: 674 - DNA, 10 bp, synthetic construct
  source 1..10: mol_type = other DNA
cggaatcggt

SEQ ID NO: 675 - DNA, 10 bp, synthetic construct
  source 1..10: mol_type = other DNA
cttgcggaga

SEQ ID NO: 676 - DNA, 10 bp, synthetic construct
  source 1..10: mol_type = other DNA
aaaaatttgg

SEQ ID NO: 677 - DNA, 10 bp, synthetic construct
  source 1..10: mol_type = other DNA
tgttttccgc

SEQ ID NO: 678 - DNA, 10 bp, synthetic construct
  source 1..10: mol_type = other DNA
atgctaggcg

SEQ ID NO: 679 - DNA, 10 bp, synthetic construct
  source 1..10: mol_type = other DNA
gactaatttc

SEQ ID NO: 680 - DNA, 10 bp, synthetic construct
  source 1..10: mol_type = other DNA
ctgtagtaac

SEQ ID NO: 681 - DNA, 10 bp, synthetic construct
  source 1..10: mol_type = other DNA
cggatgactt

SEQ ID NO: 682 - DNA, 10 bp, synthetic construct
  source 1..10: mol_type = other DNA
tcagagtgga

SEQ ID NO: 683 - DNA, 10 bp, synthetic construct
  source 1..10: mol_type = other DNA
caaaatagcg

SEQ ID NO: 684 - DNA, 10 bp, synthetic construct
  source 1..10: mol_type = other DNA
gaagaagaag

SEQ ID NO: 685 - DNA, 10 bp, synthetic construct
  source 1..10: mol_type = other DNA
cacccgcacg

SEQ ID NO: 686 - DNA, 10 bp, synthetic construct
  source 1..10: mol_type = other DNA
acgatgcccg

SEQ ID NO: 687 - DNA, 10 bp, synthetic construct
  source 1..10: mol_type = other DNA
cctactacac

SEQ ID NO: 688 - DNA, 10 bp, synthetic construct
  source 1..10: mol_type = other DNA
attgaaacaa

SEQ ID NO: 689 - DNA, 10 bp, synthetic construct
  source 1..10: mol_type = other DNA
gaccgaagat

SEQ ID NO: 690 - DNA, 10 bp, synthetic construct
  source 1..10: mol_type = other DNA
acggcctgaa

SEQ ID NO: 691 - DNA, 10 bp, synthetic construct
  source 1..10: mol_type = other DNA
aggggaggtc

SEQ ID NO: 692 - DNA, 10 bp, synthetic construct
  source 1..10: mol_type = other DNA
caatcaactt

SEQ ID NO: 693 - DNA, 10 bp, synthetic construct
  source 1..10: mol_type = other DNA
ggacaaccga

SEQ ID NO: 694 - DNA, 10 bp, synthetic construct
  source 1..10: mol_type = other DNA
tccctaaggc

SEQ ID NO: 695 - DNA, 10 bp, synthetic construct
  source 1..10: mol_type = other DNA
gttctacacg

SEQ ID NO: 696 - DNA, 10 bp, synthetic construct
  source 1..10: mol_type = other DNA
actaaccagt

SEQ ID NO: 697 - DNA, 10 bp, synthetic construct
  source 1..10: mol_type = other DNA
gaagctggat

SEQ ID NO: 698 - DNA, 10 bp, synthetic construct
  source 1..10: mol_type = other DNA
ggaaccatgg

SEQ ID NO: 699 - DNA, 10 bp, synthetic construct
  source 1..10: mol_type = other DNA
ctctacctgg

SEQ ID NO: 700 - DNA, 10 bp, synthetic construct
  source 1..10: mol_type = other DNA
taatgcctgc

SEQ ID NO: 701 - DNA, 10 bp, synthetic construct
  source 1..10: mol_type = other DNA
taaaggcaat

SEQ ID NO: 702 - DNA, 10 bp, synthetic construct
  source 1..10: mol_type = other DNA
cgcctgggaa

SEQ ID NO: 703 - DNA, 10 bp, synthetic construct
  source 1..10: mol_type = other DNA
tcttggggaa

SEQ ID NO: 704 - DNA, 10 bp, synthetic construct
  source 1..10: mol_type = other DNA
agagagagag

SEQ ID NO: 705 - DNA, 10 bp, synthetic construct
  source 1..10: mol_type = other DNA
gcgttggcgc

SEQ ID NO: 706 - DNA, 10 bp, synthetic construct
  source 1..10: mol_type = other DNA
ttacgacaga

SEQ ID NO: 707 - DNA, 10 bp, synthetic construct
  source 1..10: mol_type = other DNA
ggaactctta

SEQ ID NO: 708 - DNA, 10 bp, synthetic construct
  source 1..10: mol_type = other DNA
gattgtggag

SEQ ID NO: 709 - DNA, 10 bp, synthetic construct
  source 1..10: mol_type = other DNA
gggcactgat

SEQ ID NO: 710 - DNA, 10 bp, synthetic construct
  source 1..10: mol_type = other DNA
agacgcacca

SEQ ID NO: 711 - DNA, 10 bp, synthetic construct
  source 1..10: mol_type = other DNA
ccaattataa

SEQ ID NO: 712 - DNA, 10 bp, synthetic construct
  source 1..10: mol_type = other DNA
tagagacgca

SEQ ID NO: 713 - DNA, 10 bp, synthetic construct
  source 1..10: mol_type = other DNA
cctcttgtcg

SEQ ID NO: 714 - DNA, 10 bp, synthetic construct
  source 1..10: mol_type = other DNA
gaggaagctc

SEQ ID NO: 715 - DNA, 10 bp, synthetic construct
  source 1..10: mol_type = other DNA
agtcccgagt

SEQ ID NO: 716 - DNA, 10 bp, synthetic construct
  source 1..10: mol_type = other DNA
tgcttgcagt

SEQ ID NO: 717 - DNA, 10 bp, synthetic construct
  source 1..10: mol_type = other DNA
cccacttccc

SEQ ID NO: 718 - DNA, 10 bp, synthetic construct
  source 1..10: mol_type = other DNA
cgttgccgcg

SEQ ID NO: 719 - DNA, 10 bp, synthetic construct
  source 1..10: mol_type = other DNA
cccctggttc

SEQ ID NO: 720 - DNA, 10 bp, synthetic construct
  source 1..10: mol_type = other DNA
acgaccaata

SEQ ID NO: 721 - DNA, 10 bp, synthetic construct
  source 1..10: mol_type = other DNA
cttagggttc

SEQ ID NO: 722 - DNA, 10 bp, synthetic construct
  source 1..10: mol_type = other DNA
aaacatatca

SEQ ID NO: 723 - DNA, 10 bp, synthetic construct
  source 1..10: mol_type = other DNA
gggtcgtaga

SEQ ID NO: 724 - DNA, 10 bp, synthetic construct
  source 1..10: mol_type = other DNA
ctccgtagcg

SEQ ID NO: 725 - DNA, 10 bp, synthetic construct
  source 1..10: mol_type = other DNA
ctggtcataa

SEQ ID NO: 726 - DNA, 10 bp, synthetic construct
  source 1..10: mol_type = other DNA
ttgacagatc

SEQ ID NO: 727 - DNA, 10 bp, synthetic construct
  source 1..10: mol_type = other DNA
gagtaaagtc

SEQ ID NO: 728 - DNA, 10 bp, synthetic construct
  source 1..10: mol_type = other DNA
atatgggctt

SEQ ID NO: 729 - DNA, 10 bp, synthetic construct
  source 1..10: mol_type = other DNA
tacaactact

SEQ ID NO: 730 - DNA, 10 bp, synthetic construct
  source 1..10: mol_type = other DNA
aattcagccg

SEQ ID NO: 731 - DNA, 10 bp, synthetic construct
  source 1..10: mol_type = other DNA
gattgtacta

SEQ ID NO: 732 - DNA, 10 bp, synthetic construct
  source 1..10: mol_type = other DNA
tcgtaatgcg

SEQ ID NO: 733 - DNA, 10 bp, synthetic construct
  source 1..10: mol_type = other DNA
cgataactgc

SEQ ID NO: 734 - DNA, 10 bp, synthetic construct
  source 1..10: mol_type = other DNA
aacttggcgg

SEQ ID NO: 735 - DNA, 10 bp, synthetic construct
  source 1..10: mol_type = other DNA
cgtggatgta

SEQ ID NO: 736 - DNA, 10 bp, synthetic construct
  source 1..10: mol_type = other DNA
ccttcccgaa

SEQ ID NO: 737 - DNA, 10 bp, synthetic construct
  source 1..10: mol_type = other DNA
ctaaacccgt

SEQ ID NO: 738 - DNA, 10 bp, synthetic construct
  source 1..10: mol_type = other DNA
caacattccc

SEQ ID NO: 739 - DNA, 10 bp, synthetic construct
  source 1..10: mol_type = other DNA
cttaccctct

SEQ ID NO: 740 - DNA, 10 bp, synthetic construct
  source 1..10: mol_type = other DNA
ggaaagttct

SEQ ID NO: 741 - DNA, 10 bp, synthetic construct
  source 1..10: mol_type = other DNA
cggattggct

SEQ ID NO: 742 - DNA, 10 bp, synthetic construct
  source 1..10: mol_type = other DNA
aatgtagggc

SEQ ID NO: 743 - DNA, 10 bp, synthetic construct
  source 1..10: mol_type = other DNA
aatgaatcgc

SEQ ID NO: 744 - DNA, 10 bp, synthetic construct
  source 1..10: mol_type = other DNA
atcatacacc

SEQ ID NO: 745 - DNA, 10 bp, synthetic construct
  source 1..10: mol_type = other DNA
agttgggcag

SEQ ID NO: 746 - DNA, 10 bp, synthetic construct
  source 1..10: mol_type = other DNA
agaagaaggg

SEQ ID NO: 747 - DNA, 10 bp, synthetic construct
  source 1..10: mol_type = other DNA
gcgtgcgcta

SEQ ID NO: 748 - DNA, 10 bp, synthetic construct
  source 1..10: mol_type = other DNA
ccccgataaa

SEQ ID NO: 749 - DNA, 10 bp, synthetic construct
  source 1..10: mol_type = other DNA
taccaagtgc

SEQ ID NO: 750 - DNA, 10 bp, synthetic construct
  source 1..10: mol_type = other DNA
tgtgttttcg

SEQ ID NO: 751 - DNA, 10 bp, synthetic construct
  source 1..10: mol_type = other DNA
cccagatgtc

SEQ ID NO: 752 - DNA, 10 bp, synthetic construct
  source 1..10: mol_type = other DNA
gcgagcttcc

SEQ ID NO: 753 - DNA, 10 bp, synthetic construct
  source 1..10: mol_type = other DNA
gtgtcacgta

SEQ ID NO: 754 - DNA, 10 bp, synthetic construct
  source 1..10: mol_type = other DNA
ataggccgag

SEQ ID NO: 755 - DNA, 10 bp, synthetic construct
  source 1..10: mol_type = other DNA
gagctaccag

SEQ ID NO: 756 - DNA, 10 bp, synthetic construct
  source 1..10: mol_type = other DNA
cgcggcggag

SEQ ID NO: 757 - DNA, 10 bp, synthetic construct
  source 1..10: mol_type = other DNA
tcttgcacga

SEQ ID NO: 758 - DNA, 10 bp, synthetic construct
  source 1..10: mol_type = other DNA
tgccctaaag

SEQ ID NO: 759 - DNA, 10 bp, synthetic construct
  source 1..10: mol_type = other DNA
ttgcgctttg

SEQ ID NO: 760 - DNA, 10 bp, synthetic construct
  source 1..10: mol_type = other DNA
catataaagg

SEQ ID NO: 761 - DNA, 10 bp, synthetic construct
  source 1..10: mol_type = other DNA
aatagcgaat

SEQ ID NO: 762 - DNA, 10 bp, synthetic construct
  source 1..10: mol_type = other DNA
tacgctaagg

SEQ ID NO: 763 - DNA, 10 bp, synthetic construct
  source 1..10: mol_type = other DNA
acttagttcg

SEQ ID NO: 764 - DNA, 10 bp, synthetic construct
  source 1..10: mol_type = other DNA
cgtgcggaac

SEQ ID NO: 765 - DNA, 10 bp, synthetic construct
  source 1..10: mol_type = other DNA
acccgattcg

SEQ ID NO: 766 - DNA, 10 bp, synthetic construct
  source 1..10: mol_type = other DNA
tgcagagttt

SEQ ID NO: 767 - DNA, 10 bp, synthetic construct
  source 1..10: mol_type = other DNA
gaatcattag

SEQ ID NO: 768 - DNA, 10 bp, synthetic construct
  source 1..10: mol_type = other DNA
agtacactgg

SEQ ID NO: 769 - DNA, 10 bp, synthetic construct
  source 1..10: mol_type = other DNA
ttgtgcggtt

SEQ ID NO: 770 - DNA, 10 bp, synthetic construct
  source 1..10: mol_type = other DNA
atgacatgca

SEQ ID NO: 771 - DNA, 10 bp, synthetic construct
  source 1..10: mol_type = other DNA
ttctcggacg

SEQ ID NO: 772 - DNA, 10 bp, synthetic construct
  source 1..10: mol_type = other DNA
agattgaaga

SEQ ID NO: 773 - DNA, 10 bp, synthetic construct
  source 1..10: mol_type = other DNA
ggcggactgt

SEQ ID NO: 774 - DNA, 10 bp, synthetic construct
  source 1..10: mol_type = other DNA
tttatggtaa

SEQ ID NO: 775 - DNA, 10 bp, synthetic construct
  source 1..10: mol_type = other DNA
cagtagggtg

SEQ ID NO: 776 - DNA, 10 bp, synthetic construct
  source 1..10: mol_type = other DNA
gacaggcaag

SEQ ID NO: 777 - DNA, 10 bp, synthetic construct
  source 1..10: mol_type = other DNA
gatgtgtcgt

SEQ ID NO: 778 - DNA, 10 bp, synthetic construct
  source 1..10: mol_type = other DNA
acttgacgga

SEQ ID NO: 779 - DNA, 10 bp, synthetic construct
  source 1..10: mol_type = other DNA
aagtccgaaa

SEQ ID NO: 780 - DNA, 10 bp, synthetic construct
  source 1..10: mol_type = other DNA
tgggtgtagg

SEQ ID NO: 781 - DNA, 10 bp, synthetic construct
  source 1..10: mol_type = other DNA
acttaccgcg

SEQ ID NO: 782 - DNA, 10 bp, synthetic construct
  source 1..10: mol_type = other DNA
ctgtgcaccc

SEQ ID NO: 783 - DNA, 10 bp, synthetic construct
  source 1..10: mol_type = other DNA
attgctctct

SEQ ID NO: 784 - DNA, 10 bp, synthetic construct
  source 1..10: mol_type = other DNA
cagaagacaa

SEQ ID NO: 785 - DNA, 10 bp, synthetic construct
  source 1..10: mol_type = other DNA
ttacgctata

SEQ ID NO: 786 - DNA, 10 bp, synthetic construct
  source 1..10: mol_type = other DNA
acgtggaaat

SEQ ID NO: 787 - DNA, 10 bp, synthetic construct
  source 1..10: mol_type = other DNA
tgaggctggt

SEQ ID NO: 788 - DNA, 10 bp, synthetic construct
  source 1..10: mol_type = other DNA
attatgagat

SEQ ID NO: 789 - DNA, 10 bp, synthetic construct
  source 1..10: mol_type = other DNA
gacttgtagt

SEQ ID NO: 790 - DNA, 10 bp, synthetic construct
  source 1..10: mol_type = other DNA
tcgctgagga

SEQ ID NO: 791 - DNA, 10 bp, synthetic construct
  source 1..10: mol_type = other DNA
cccaactcta

SEQ ID NO: 792 - DNA, 10 bp, synthetic construct
  source 1..10: mol_type = other DNA
gatagggagg

SEQ ID NO: 793 - DNA, 10 bp, synthetic construct
  source 1..10: mol_type = other DNA
tagaaatcag

SEQ ID NO: 794 - DNA, 10 bp, synthetic construct
  source 1..10: mol_type = other DNA
gtcgctagaa

SEQ ID NO: 795 - DNA, 10 bp, synthetic construct
  source 1..10: mol_type = other DNA
aaaatagaaa

SEQ ID NO: 796 - DNA, 10 bp, synthetic construct
  source 1..10: mol_type = other DNA
gctcctgggt

SEQ ID NO: 797 - DNA, 10 bp, synthetic construct
  source 1..10: mol_type = other DNA
cgcgctcgcg

SEQ ID NO: 798 - DNA, 10 bp, synthetic construct
  source 1..10: mol_type = other DNA
ggcaaacgca

SEQ ID NO: 799 - DNA, 10 bp, synthetic construct
  source 1..10: mol_type = other DNA
tttactacct

SEQ ID NO: 800 - DNA, 10 bp, synthetic construct
  source 1..10: mol_type = other DNA
atcctaaact

SEQ ID NO: 801 - DNA, 10 bp, synthetic construct
  source 1..10: mol_type = other DNA
ctccgtatgt

SEQ ID NO: 802 - DNA, 10 bp, synthetic construct
  source 1..10: mol_type = other DNA
tatcgtccag

SEQ ID NO: 803 - DNA, 10 bp, synthetic construct
  source 1..10: mol_type = other DNA
gccggcggta

SEQ ID NO: 804 - DNA, 10 bp, synthetic construct
  source 1..10: mol_type = other DNA
tgctccattt

SEQ ID NO: 805 - DNA, 10 bp, synthetic construct
  source 1..10: mol_type = other DNA
tggctgttgt

SEQ ID NO: 806 - DNA, 10 bp, synthetic construct
  source 1..10: mol_type = other DNA
tactgcgcaa

SEQ ID NO: 807 - DNA, 10 bp, synthetic construct
  source 1..10: mol_type = other DNA
tatacggctt

SEQ ID NO: 808 - DNA, 10 bp, synthetic construct
  source 1..10: mol_type = other DNA
ggttattacc

SEQ ID NO: 809 - DNA, 10 bp, synthetic construct
  source 1..10: mol_type = other DNA
atcaggagga

SEQ ID NO: 810 - DNA, 10 bp, synthetic construct
  source 1..10: mol_type = other DNA
ctattgccag

SEQ ID NO: 811 - DNA, 10 bp, synthetic construct
  source 1..10: mol_type = other DNA
acgtacacac

SEQ ID NO: 812 - DNA, 10 bp, synthetic construct
  source 1..10: mol_type = other DNA
cagcctagct

SEQ ID NO: 813 - DNA, 10 bp, synthetic construct
  source 1..10: mol_type = other DNA
gaaaaacaac

SEQ ID NO: 814 - DNA, 10 bp, synthetic construct
  source 1..10: mol_type = other DNA
cgttcagtta

SEQ ID NO: 815 - DNA, 10 bp, synthetic construct
  source 1..10: mol_type = other DNA
caatcagaat

SEQ ID NO: 816 - DNA, 10 bp, synthetic construct
  source 1..10: mol_type = other DNA
gggctactct

SEQ ID NO: 817 - DNA, 10 bp, synthetic construct
  source 1..10: mol_type = other DNA
ccccattggg

SEQ ID NO: 818 - DNA, 10 bp, synthetic construct
  source 1..10: mol_type = other DNA
tagggaacgg

SEQ ID NO: 819 - DNA, 10 bp, synthetic construct
  source 1..10: mol_type = other DNA
cagctgatac

SEQ ID NO: 820 - DNA, 10 bp, synthetic construct
  source 1..10: mol_type = other DNA
attcctgtga

SEQ ID NO: 821 - DNA, 10 bp, synthetic construct
  source 1..10: mol_type = other DNA
tcagagccgt

SEQ ID NO: 822 - DNA, 10 bp, synthetic construct
  source 1..10: mol_type = other DNA
catgaaaagc

SEQ ID NO: 823 - DNA, 10 bp, synthetic construct
  source 1..10: mol_type = other DNA
tgacctgtga

SEQ ID NO: 824 - DNA, 10 bp, synthetic construct
  source 1..10: mol_type = other DNA
gcattagcag

SEQ ID NO: 825 - DNA, 10 bp, synthetic construct
  source 1..10: mol_type = other DNA
gacagaacca

SEQ ID NO: 826 - DNA, 10 bp, synthetic construct
  source 1..10: mol_type = other DNA
tccagtatat

SEQ ID NO: 827 - DNA, 10 bp, synthetic construct
  source 1..10: mol_type = other DNA
tgttccgcta

SEQ ID NO: 828 - DNA, 10 bp, synthetic construct
  source 1..10: mol_type = other DNA
gatatccatt

SEQ ID NO: 829 - DNA, 10 bp, synthetic construct
  source 1..10: mol_type = other DNA
catatggacc

SEQ ID NO: 830 - DNA, 10 bp, synthetic construct
  source 1..10: mol_type = other DNA
gatatagtaa

SEQ ID NO: 831 - DNA, 10 bp, synthetic construct
  source 1..10: mol_type = other DNA
cacctttttt

SEQ ID NO: 832 - DNA, 10 bp, synthetic construct
  source 1..10: mol_type = other DNA
agcttgcggg

SEQ ID NO: 833 - DNA, 10 bp, synthetic construct
  source 1..10: mol_type = other DNA
cgcacaggga

SEQ ID NO: 834 - DNA, 10 bp, synthetic construct
  source 1..10: mol_type = other DNA
tctgggtgct

SEQ ID NO: 835 - DNA, 10 bp, synthetic construct
  source 1..10: mol_type = other DNA
tgagtcgttt

SEQ ID NO: 836 - DNA, 10 bp, synthetic construct
  source 1..10: mol_type = other DNA
ttacaatgtg

SEQ ID NO: 837 - DNA, 10 bp, synthetic construct
  source 1..10: mol_type = other DNA
cttgcaaaca

SEQ ID NO: 838 - DNA, 10 bp, synthetic construct
  source 1..10: mol_type = other DNA
tgtcgagctg

SEQ ID NO: 839 - DNA, 10 bp, synthetic construct
  source 1..10: mol_type = other DNA
actttaacct

SEQ ID NO: 840 - DNA, 10 bp, synthetic construct
  source 1..10: mol_type = other DNA
atataagtgc

SEQ ID NO: 841 - DNA, 10 bp, synthetic construct
  source 1..10: mol_type = other DNA
ggaagggcgt

SEQ ID NO: 842 - DNA, 10 bp, synthetic construct
  source 1..10: mol_type = other DNA
tttgacttga

SEQ ID NO: 843 - DNA, 10 bp, synthetic construct
  source 1..10: mol_type = other DNA
gtataaacgg

SEQ ID NO: 844 - DNA, 10 bp, synthetic construct
  source 1..10: mol_type = other DNA
taaccggatg

SEQ ID NO: 845 - DNA, 10 bp, synthetic construct
  source 1..10: mol_type = other DNA
ttctcatcag

SEQ ID NO: 846 - DNA, 10 bp, synthetic construct
  source 1..10: mol_type = other DNA
ctcggttacg

SEQ ID NO: 847 - DNA, 10 bp, synthetic construct
  source 1..10: mol_type = other DNA
atatggttct

SEQ ID NO: 848 - DNA, 10 bp, synthetic construct
  source 1..10: mol_type = other DNA
cgcccccgaa

SEQ ID NO: 849 - DNA, 10 bp, synthetic construct
  source 1..10: mol_type = other DNA
acctcgatcg

SEQ ID NO: 850 - DNA, 10 bp, synthetic construct
  source 1..10: mol_type = other DNA
ctcgaataat

SEQ ID NO: 851 - DNA, 10 bp, synthetic construct
  source 1..10: mol_type = other DNA
gcccgagctt

SEQ ID NO: 852 - DNA, 10 bp, synthetic construct
  source 1..10: mol_type = other DNA
aacagtcaac

SEQ ID NO: 853 - DNA, 10 bp, synthetic construct
  source 1..10: mol_type = other DNA
ctggaacctc

SEQ ID NO: 854 - DNA, 10 bp, synthetic construct
  source 1..10: mol_type = other DNA
aataacgggg

SEQ ID NO: 855 - DNA, 10 bp, synthetic construct
  source 1..10: mol_type = other DNA
acgccccact

SEQ ID NO: 856 - DNA, 10 bp, synthetic construct
  source 1..10: mol_type = other DNA
ggcaacatga

SEQ ID NO: 857 - DNA, 10 bp, synthetic construct
  source 1..10: mol_type = other DNA
gctatttcgc

SEQ ID NO: 858 - DNA, 10 bp, synthetic construct
  source 1..10: mol_type = other DNA
ttccacttta

SEQ ID NO: 859 - DNA, 10 bp, synthetic construct
  source 1..10: mol_type = other DNA
gccgatggat

SEQ ID NO: 860 - DNA, 10 bp, synthetic construct
  source 1..10: mol_type = other DNA
aagttggtaa

SEQ ID NO: 861 - DNA, 10 bp, synthetic construct
  source 1..10: mol_type = other DNA
cactagctag

SEQ ID NO: 862 - DNA, 10 bp, synthetic construct
  source 1..10: mol_type = other DNA
acatgcccct

SEQ ID NO: 863 - DNA, 10 bp, synthetic construct
  source 1..10: mol_type = other DNA
ttcattactc

SEQ ID NO: 864 - DNA, 10 bp, synthetic construct
  source 1..10: mol_type = other DNA
ggtttaatat

SEQ ID NO: 865 - DNA, 10 bp, synthetic construct
  source 1..10: mol_type = other DNA
cctgcagtga

SEQ ID NO: 866 - DNA, 10 bp, synthetic construct
  source 1..10: mol_type = other DNA
tctttaagtt

SEQ ID NO: 867 - DNA, 10 bp, synthetic construct
  source 1..10: mol_type = other DNA
tggcgatcga

SEQ ID NO: 868 - DNA, 10 bp, synthetic construct
  source 1..10: mol_type = other DNA
ctttttagct

SEQ ID NO: 869 - DNA, 10 bp, synthetic construct
  source 1..10: mol_type = other DNA
cccagtctct

SEQ ID NO: 870 - DNA, 10 bp, synthetic construct
  source 1..10: mol_type = other DNA
aaatgtttcg

SEQ ID NO: 871 - DNA, 10 bp, synthetic construct
  source 1..10: mol_type = other DNA
atataagacg

SEQ ID NO: 872 - DNA, 10 bp, synthetic construct
  source 1..10: mol_type = other DNA
tcactttaca

SEQ ID NO: 873 - DNA, 10 bp, synthetic construct
  source 1..10: mol_type = other DNA
cctggcgccc

SEQ ID NO: 874 - DNA, 10 bp, synthetic construct
  source 1..10: mol_type = other DNA
ggattactgg

SEQ ID NO: 875 - DNA, 10 bp, synthetic construct
  source 1..10: mol_type = other DNA
gaatgatctt

SEQ ID NO: 876 - DNA, 10 bp, synthetic construct
  source 1..10: mol_type = other DNA
gctcggatcg

SEQ ID NO: 877 - DNA, 10 bp, synthetic construct
  source 1..10: mol_type = other DNA
cagctgcgag

SEQ ID NO: 878 - DNA, 10 bp, synthetic construct
  source 1..10: mol_type = other DNA
acccttacta

SEQ ID NO: 879 - DNA, 10 bp, synthetic construct
  source 1..10: mol_type = other DNA
aggtgaaact

SEQ ID NO: 880 - DNA, 10 bp, synthetic construct
  source 1..10: mol_type = other DNA
cgaatttgat

SEQ ID NO: 881 - DNA, 10 bp, synthetic construct
  source 1..10: mol_type = other DNA
cgctgtgcgg

SEQ ID NO: 882 - DNA, 10 bp, synthetic construct
  source 1..10: mol_type = other DNA
ttaccgcacc

SEQ ID NO: 883 - DNA, 10 bp, synthetic construct
  source 1..10: mol_type = other DNA
ggaatcttaa

SEQ ID NO: 884 - DNA, 10 bp, synthetic construct
  source 1..10: mol_type = other DNA
ctcaacaccc

SEQ ID NO: 885 - DNA, 10 bp, synthetic construct
  source 1..10: mol_type = other DNA
cgtgcccttg

SEQ ID NO: 886 - DNA, 10 bp, synthetic construct
  source 1..10: mol_type = other DNA
gcaggctcga

SEQ ID NO: 887 - DNA, 10 bp, synthetic construct
  source 1..10: mol_type = other DNA
accaacgaag

SEQ ID NO: 888 - DNA, 10 bp, synthetic construct
  source 1..10: mol_type = other DNA
cctgtaattt

SEQ ID NO: 889 - DNA, 10 bp, synthetic construct
  source 1..10: mol_type = other DNA
gggtgggatg

SEQ ID NO: 890 - DNA, 10 bp, synthetic construct
  source 1..10: mol_type = other DNA
ttgctcaccg

SEQ ID NO: 891 - DNA, 10 bp, synthetic construct
  source 1..10: mol_type = other DNA
ttacgaccac

SEQ ID NO: 892 - DNA, 10 bp, synthetic construct
  source 1..10: mol_type = other DNA
ttttctaacc

SEQ ID NO: 893 - DNA, 10 bp, synthetic construct
  source 1..10: mol_type = other DNA
gctttagata

SEQ ID NO: 894 - DNA, 10 bp, synthetic construct
  source 1..10: mol_type = other DNA
cacgtattgg

SEQ ID NO: 895 - DNA, 10 bp, synthetic construct
  source 1..10: mol_type = other DNA
aaatatctcc

SEQ ID NO: 896 - DNA, 10 bp, synthetic construct
  source 1..10: mol_type = other DNA
gctggaaaac

SEQ ID NO: 897 - DNA, 10 bp, synthetic construct
  source 1..10: mol_type = other DNA
gagcgcatta

SEQ ID NO: 898 - DNA, 10 bp, synthetic construct
  source 1..10: mol_type = other DNA
gtggaggggt

SEQ ID NO: 899 - DNA, 10 bp, synthetic construct
  source 1..10: mol_type = other DNA
tccactggga

SEQ ID NO: 900 - DNA, 10 bp, synthetic construct
  source 1..10: mol_type = other DNA
caatagcgga

SEQ ID NO: 901 - DNA, 10 bp, synthetic construct
  source 1..10: mol_type = other DNA
catctagttt

SEQ ID NO: 902 - DNA, 10 bp, synthetic construct
  source 1..10: mol_type = other DNA
gaagttccgg

SEQ ID NO: 903 - DNA, 10 bp, synthetic construct
  source 1..10: mol_type = other DNA
agcgagattc

SEQ ID NO: 904 - DNA, 10 bp, synthetic construct
  source 1..10: mol_type = other DNA
ttaaggtcgg

SEQ ID NO: 905 - DNA, 10 bp, synthetic construct
  source 1..10: mol_type = other DNA
aatggttagg

SEQ ID NO: 906 - DNA, 10 bp, synthetic construct
  source 1..10: mol_type = other DNA
cgttattata

SEQ ID NO: 907 - DNA, 10 bp, synthetic construct
  source 1..10: mol_type = other DNA
acggaaagga

SEQ ID NO: 908 - DNA, 10 bp, synthetic construct
  source 1..10: mol_type = other DNA
ccttgtcccg

SEQ ID NO: 909 - DNA, 10 bp, synthetic construct
  source 1..10: mol_type = other DNA
atactttttt

SEQ ID NO: 910 - DNA, 10 bp, synthetic construct
  source 1..10: mol_type = other DNA
ctgggtctgg

SEQ ID NO: 911 - DNA, 10 bp, synthetic construct
  source 1..10: mol_type = other DNA
aaccattgcg

SEQ ID NO: 912 - DNA, 10 bp, synthetic construct
  source 1..10: mol_type = other DNA
agaccgggcc

SEQ ID NO: 913 - DNA, 10 bp, synthetic construct
  source 1..10: mol_type = other DNA
tgggacacac

SEQ ID NO: 914 - DNA, 10 bp, synthetic construct
  source 1..10: mol_type = other DNA
tgcgcagttg

SEQ ID NO: 915 - DNA, 10 bp, synthetic construct
  source 1..10: mol_type = other DNA
cgttcgcctt

SEQ ID NO: 916 - DNA, 10 bp, synthetic construct
  source 1..10: mol_type = other DNA
tctcactcgt

SEQ ID NO: 917 - DNA, 10 bp, synthetic construct
  source 1..10: mol_type = other DNA
acaccgacgt

SEQ ID NO: 918 - DNA, 10 bp, synthetic construct
  source 1..10: mol_type = other DNA
ttcagcccct

SEQ ID NO: 919 - DNA, 10 bp, synthetic construct
  source 1..10: mol_type = other DNA
aggcgactaa

SEQ ID NO: 920 - DNA, 10 bp, synthetic construct
  source 1..10: mol_type = other DNA
tgctatcaag

SEQ ID NO: 921 - DNA, 10 bp, synthetic construct
  source 1..10: mol_type = other DNA
gtccagtagc

SEQ ID NO: 922 - DNA, 10 bp, synthetic construct
  source 1..10: mol_type = other DNA
cgtgtgggcg

SEQ ID NO: 923 - DNA, 10 bp, synthetic construct
  source 1..10: mol_type = other DNA
gtggttctcc

SEQ ID NO: 924 - DNA, 10 bp, synthetic construct
  source 1..10: mol_type = other DNA
gcagccgacg

SEQ ID NO: 925 - DNA, 10 bp, synthetic construct
  source 1..10: mol_type = other DNA
gctgtccacg

SEQ ID NO: 926 - DNA, 10 bp, synthetic construct
  source 1..10: mol_type = other DNA
cgacactcat

SEQ ID NO: 927 - DNA, 10 bp, synthetic construct
  source 1..10: mol_type = other DNA
catggcacct

SEQ ID NO: 928 - DNA, 10 bp, synthetic construct
  source 1..10: mol_type = other DNA
tgtgacgtgt

SEQ ID NO: 929 - DNA, 10 bp, synthetic construct
  source 1..10: mol_type = other DNA
tttggactaa

SEQ ID NO: 930 - DNA, 10 bp, synthetic construct
  source 1..10: mol_type = other DNA
ttcatgcccg

SEQ ID NO: 931 - DNA, 10 bp, synthetic construct
  source 1..10: mol_type = other DNA
ttgatcgtgg

SEQ ID NO: 932 - DNA, 10 bp, synthetic construct
  source 1..10: mol_type = other DNA
tagcatagga

SEQ ID NO: 933 - DNA, 10 bp, synthetic construct
  source 1..10: mol_type = other DNA
gtagttgcaa

SEQ ID NO: 934 - DNA, 10 bp, synthetic construct
  source 1..10: mol_type = other DNA
gggacagcta

SEQ ID NO: 935 - DNA, 10 bp, synthetic construct
  source 1..10: mol_type = other DNA
aaacccccaa

SEQ ID NO: 936 - DNA, 10 bp, synthetic construct
  source 1..10: mol_type = other DNA
actctcacaa

SEQ ID NO: 937 - DNA, 10 bp, synthetic construct
  source 1..10: mol_type = other DNA
atcattgcca

SEQ ID NO: 938 - DNA, 10 bp, synthetic construct
  source 1..10: mol_type = other DNA
ccagtttgcg

SEQ ID NO: 939 - DNA, 10 bp, synthetic construct
  source 1..10: mol_type = other DNA
acattagtca

SEQ ID NO: 940 - DNA, 10 bp, synthetic construct
  source 1..10: mol_type = other DNA
ctccagggta

SEQ ID NO: 941 - DNA, 10 bp, synthetic construct
  source 1..10: mol_type = other DNA
gaagggccaa

SEQ ID NO: 942 - DNA, 10 bp, synthetic construct
  source 1..10: mol_type = other DNA
cagtctcccc

SEQ ID NO: 943 - DNA, 10 bp, synthetic construct
  source 1..10: mol_type = other DNA
gagacattcc

SEQ ID NO: 944 - DNA, 10 bp, synthetic construct
  source 1..10: mol_type = other DNA
aacggtgttg

SEQ ID NO: 945 - DNA, 10 bp, synthetic construct
  source 1..10: mol_type = other DNA
agcattatca

SEQ ID NO: 946 - DNA, 10 bp, synthetic construct
  source 1..10: mol_type = other DNA
ctataccgag

SEQ ID NO: 947 - DNA, 10 bp, synthetic construct
  source 1..10: mol_type = other DNA
aactggatca

SEQ ID NO: 948 - DNA, 10 bp, synthetic construct
  source 1..10: mol_type = other DNA
gtcttgtcgg

SEQ ID NO: 949 - DNA, 10 bp, synthetic construct
  source 1..10: mol_type = other DNA
gacgagccgc

SEQ ID NO: 950 - DNA, 10 bp, synthetic construct
  source 1..10: mol_type = other DNA
ggaacactgt

SEQ ID NO: 951 - DNA, 10 bp, synthetic construct
  source 1..10: mol_type = other DNA
taaatgcgtt

SEQ ID NO: 952 - DNA, 10 bp, synthetic construct
  source 1..10: mol_type = other DNA
gcgaacacag

SEQ ID NO: 953 - DNA, 10 bp, synthetic construct
  source 1..10: mol_type = other DNA
ttctctcaac

SEQ ID NO: 954 - DNA, 10 bp, synthetic construct
  source 1..10: mol_type = other DNA
gtcgtactga

SEQ ID NO: 955 - DNA, 10 bp, synthetic construct
  source 1..10: mol_type = other DNA
tgtggcgtaa

SEQ ID NO: 956 - DNA, 10 bp, synthetic construct
  source 1..10: mol_type = other DNA
tgagcggcgt

SEQ ID NO: 957 - DNA, 10 bp, synthetic construct
  source 1..10: mol_type = other DNA
cctcgtgaac

SEQ ID NO: 958 - DNA, 10 bp, synthetic construct
  source 1..10: mol_type = other DNA
gagcaatgaa

SEQ ID NO: 959 - DNA, 10 bp, synthetic construct
  source 1..10: mol_type = other DNA
cgagacctaa

SEQ ID NO: 960 - DNA, 10 bp, synthetic construct
  source 1..10: mol_type = other DNA
aactgagcgc

SEQ ID NO: 961 - DNA, 10 bp, synthetic construct
  source 1..10: mol_type = other DNA
taaagctcgt

SEQ ID NO: 962 - DNA, 10 bp, synthetic construct
  source 1..10: mol_type = other DNA
ctctttacgt

SEQ ID NO: 963 - DNA, 10 bp, synthetic construct
  source 1..10: mol_type = other DNA
ccccgtggaa

SEQ ID NO: 964 - DNA, 10 bp, synthetic construct
  source 1..10: mol_type = other DNA
tcggttcgtc

SEQ ID NO: 965 - DNA, 10 bp, synthetic construct
  source 1..10: mol_type = other DNA
ctgcttacac

SEQ ID NO: 966 - DNA, 10 bp, synthetic construct
  source 1..10: mol_type = other DNA
acaccgtaat

SEQ ID NO: 967 - DNA, 10 bp, synthetic construct
  source 1..10: mol_type = other DNA
cctggtcggc

SEQ ID NO: 968 - DNA, 10 bp, synthetic construct
  source 1..10: mol_type = other DNA
ggttatttgg

SEQ ID NO: 969 - DNA, 10 bp, synthetic construct
  source 1..10: mol_type = other DNA
gcaactgagt

SEQ ID NO: 970 - DNA, 10 bp, synthetic construct
  source 1..10: mol_type = other DNA
ataaggcctc

SEQ ID NO: 971 - DNA, 10 bp, synthetic construct
  source 1..10: mol_type = other DNA
cgtgcgaagg

SEQ ID NO: 972 - DNA, 10 bp, synthetic construct
  source 1..10: mol_type = other DNA
gtcacacact

SEQ ID NO: 973 - DNA, 10 bp, synthetic construct
  source 1..10: mol_type = other DNA
catacggcaa

SEQ ID NO: 974 - DNA, 10 bp, synthetic construct
  source 1..10: mol_type = other DNA
gaactgccca

SEQ ID NO: 975 - DNA, 10 bp, synthetic construct
  source 1..10: mol_type = other DNA
aatatgtgaa

SEQ ID NO: 976 - DNA, 10 bp, synthetic construct
  source 1..10: mol_type = other DNA
ccgatcctgt

SEQ ID NO: 977 - DNA, 10 bp, synthetic construct
  source 1..10: mol_type = other DNA
caaagagcct

SEQ ID NO: 978 - DNA, 10 bp, synthetic construct
  source 1..10: mol_type = other DNA
taacttagag

SEQ ID NO: 979 - DNA, 10 bp, synthetic construct
  source 1..10: mol_type = other DNA
cagcatgtag

SEQ ID NO: 980 - DNA, 10 bp, synthetic construct
  source 1..10: mol_type = other DNA
ccccatgcag

SEQ ID NO: 981 - DNA, 10 bp, synthetic construct
  source 1..10: mol_type = other DNA
tctgaaccac

SEQ ID NO: 982 - DNA, 10 bp, synthetic construct
  source 1..10: mol_type = other DNA
gcgtgcaaaa

SEQ ID NO: 983 - DNA, 10 bp, synthetic construct
  source 1..10: mol_type = other DNA
gctagtaccg

SEQ ID NO: 984 - DNA, 10 bp, synthetic construct
  source 1..10: mol_type = other DNA
tttcccgcgc

SEQ ID NO: 985 - DNA, 10 bp, synthetic construct
  source 1..10: mol_type = other DNA
ccttagtagg

SEQ ID NO: 986 - DNA, 10 bp, synthetic construct
  source 1..10: mol_type = other DNA
ttgtgtcttg

SEQ ID NO: 987 - DNA, 10 bp, synthetic construct
  source 1..10: mol_type = other DNA
gcaacgaagc

SEQ ID NO: 988 - DNA, 10 bp, synthetic construct
  source 1..10: mol_type = other DNA
tgaaaccctt

SEQ ID NO: 989 - DNA, 10 bp, synthetic construct
  source 1..10: mol_type = other DNA
ttctacgatc

SEQ ID NO: 990 - DNA, 10 bp, synthetic construct
  source 1..10: mol_type = other DNA
attaaaggtg

SEQ ID NO: 991 - DNA, 10 bp, synthetic construct
  source 1..10: mol_type = other DNA
tatctaacgg

SEQ ID NO: 992 - DNA, 10 bp, synthetic construct
  source 1..10: mol_type = other DNA
agtgctcctg

SEQ ID NO: 993 - DNA, 10 bp, synthetic construct
  source 1..10: mol_type = other DNA
ccgtccctct

SEQ ID NO: 994 - DNA, 10 bp, synthetic construct
  source 1..10: mol_type = other DNA
ctaacgagcg

SEQ ID NO: 995 - DNA, 10 bp, synthetic construct
  source 1..10: mol_type = other DNA
aagtccggct

SEQ ID NO: 996 - DNA, 10 bp, synthetic construct
  source 1..10: mol_type = other DNA
ggcgtataag

SEQ ID NO: 997 - DNA, 10 bp, synthetic construct
  source 1..10: mol_type = other DNA
agatattagg

SEQ ID NO: 998 - DNA, 10 bp, synthetic construct
  source 1..10: mol_type = other DNA
tcctaacagc

SEQ ID NO: 999 - DNA, 10 bp, synthetic construct
  source 1..10: mol_type = other DNA
gaggatacgc

SEQ ID NO: 1000 - DNA, 10 bp, synthetic construct
  source 1..10: mol_type = other DNA
cgctctttaa

SEQ ID NO: 1001 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
agacgtgtgctcttccgatct

SEQ ID NO: 1002 - DNA, 20 bp, synthetic construct
  source 1..20: mol_type = other DNA
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
agatcggaagagcgtcgtgt

SEQ ID NO: 1003 - DNA, 61 bp, synthetic construct
  source 1..61: mol_type = other DNA
  modified_base 1: mod_base = OTHER; note = 5' Amino Modifier C6 modified nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  modified_base 58: mod_base = OTHER; note = Nucleotide with phosphorothioate linkage
  modified_base 59: mod_base = OTHER; note = Nucleotide with phosphorothioate linkage
  modified_base 60: mod_base = OTHER; note = Nucleotide with phosphorothioate linkage
agacgtgtgctcttccgatctgctacataatnnnnnnnnnnagatcggaagagcgtcgtgt